(19) United States
(12) Patent Application Publication
(10) Pub. No.: US 2025/0291251 A1
(43) Pub. Date: Sep. 18, 2025

(54) RESIST COMPOSITION AND PATTERN FORMING PROCESS

(71) Applicant: Shin-Etsu Chemical Co., Ltd., Tokyo (JP)

(72) Inventors: Masaki Ohashi, Joetsu-shi (JP); Masayoshi Sagehashi, Joetsu-shi (JP); Yutaro Otomo, Joetsu-shi (JP)

(73) Assignee: Shin-Etsu Chemical Co., Ltd., Tokyo (JP)

(21) Appl. No.: 19/070,239

(22) Filed: Mar. 4, 2025

(30) Foreign Application Priority Data
Mar. 14, 2024 (JP) 2024-039577

(51) Int. Cl. G03F 7/039; C07C 69/16; G03F 7/004

(52) U.S. Cl. CPC G03F 7/0397; C07C 69/16; G03F 7/0046

(57) ABSTRACT

The resist composition exhibits a high contrast, improved maximum resolution, and satisfactory sensitivity and line width roughness (LWR) when processed by lithography using high-energy radiation, especially electron beam (EB) and EUV, and a pattern forming process using the resist composition. The resist composition comprising (A) a base polymer containing a polymer comprising repeat units having an acid labile group and repeat units having a carboxy group, (B) an organic solvent, and (C) a hypervalent iodine compound having formula (1):

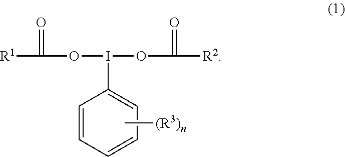

### CROSS-REFERENCE TO RELATED APPLICATIONS

This non-provisional application claims priority under 35 U.S.C. § 119(a) on Patent Application No. 2024-039577 filed in Japan on Mar. 14, 2024, the entire contents of which are hereby incorporated by reference.

### TECHNICAL FIELD

This invention relates to a resist composition and a pattern forming process.

### BACKGROUND ART

To meet the demand for higher integration density and operating speed of LSIs, the effort to reduce the pattern rule is in rapid progress. As the use of 5G high-speed communications and artificial intelligence (AI) is widely spreading, high-performance devices are needed for their processing. As the advanced miniaturization technology, manufacturing of microelectronic devices at the 5-nm node by the lithography using extreme ultraviolet (EUV) of wavelength 13.5 nm has been implemented in a mass scale. Studies are made on the application of EUV lithography to 3-nm node devices of the next generation and 2-nm node devices of the next-but-one generation.

As the feature size reduces, image blurs due to acid diffusion become a problem. To insure resolution for fine patterns with a processing dimension of 45 nm et seq., not only an improvement in dissolution contrast is important as previously reported, but the control of acid diffusion is also important as reported in Non-Patent Document 1. Since chemically amplified resist compositions are designed such that sensitivity and contrast are enhanced by acid diffusion, an attempt to minimize acid diffusion by reducing the temperature and/or time of post exposure bake (PEB) fails, resulting in drastic reductions of sensitivity and contrast.

The addition of an acid generator capable of generating a bulky acid is an effective means for controlling acid diffusion. A design has also been proposed which suppresses acid diffusion by incorporating repeat units derived from an onium salt having a polymerizable unsaturated bond in a polymer. Since this polymer functions as an acid generator, it is referred to as polymer-bound acid generator. Patent Document 1 discloses a polymer-bound acid generator capable of generating a specific fluorosulfonic acid from a base polymer.

In 3-nm node devices or 2-nm node devices, the fine pattern formation technology using the EUV lithography is required, and in such a region, sufficient resolution cannot be obtained only by the acid diffusion control. For example, in line-and-space patterns, a pattern defect such as a bridge between patterns or a scum at the bottom is a problem, which is caused by insufficient contrast or the like.

To increase the contrast, an attempt has been made to crosslink polymer chains by an acid-decomposable crosslinking group. The molecular weight can be increased in advance by crosslinking, and crosslinking in the exposed region can be decomposed by the acid generated upon exposure. Patent Document 2 discloses a crosslinked polymer obtained by causing a reaction between a unit having a carboxy group or hydroxy group and a divinyl ether unit.

The crosslinked polymer produced by crosslinking polymer chains has a very high molecular weight, and when the crosslinked polymer is stored as a resist solution for a long time, agglomeration of polymer particles occurs, leading to a problem of more defects.

### CITATION LIST

Patent Document 1: JP-A 2008-133448
Patent Document 2: JP 5562651
Patent Document 3: JP-A 2015-180928
Patent Document 4: JP-A 2018-095853
Non-Patent Document 1: SPIE Vol. 6520 65203L-1 (2007)

### SUMMARY OF THE INVENTION

An object of the invention is to provide a resist composition which exhibits a high contrast, improved maximum resolution, and satisfactory sensitivity and line width roughness (LWR) when processed by lithography using high-energy radiation, especially electron beam (EB) and EUV, and a pattern forming process using the resist composition.

The inventors have found that a resist composition comprising a base polymer containing a polymer comprising repeat units having an acid labile group and repeat units having a carboxy group, an organic solvent, and a predetermined hypervalent iodine compound increases contrast, exhibits improved maximum resolution, and is quite useful in precise micropatterning.

The invention provides a resist composition and a pattern forming process described below.

In one aspect, the invention provides a resist composition comprising (A) a base polymer containing a polymer comprising repeat units having an acid labile group and repeat units having a carboxy group, (B) an organic solvent, and (C) a hypervalent iodine compound having formula (1):

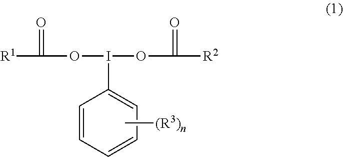

wherein n is an integer of 0 to 5,
R¹ and R² are each independently halogen or a C₁-C₁₀ hydrocarbyl group which may contain a heteroatom, R¹ and R² may bond together to form a ring with the carbon atoms to which they are attached and the intervenient atoms, and
R³ is halogen or a C₁-C₄₀ hydrocarbyl group which may contain a heteroatom.

In a preferred embodiment, the repeat unit having an acid labile group is represented by formula (a1) or (a2):

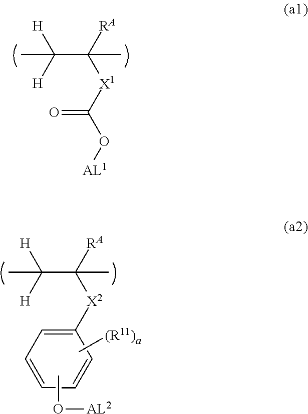

wherein a is an integer of 0 to 4,
R^{A} is each independently hydrogen, fluorine, methyl or trifluoromethyl,
X¹ is a single bond, phenylene group, naphthylene group or *—C(═O)—O—X¹¹—, X¹¹ is a C₁-C₁₀ saturated hydrocarbylene group which may contain at least one selected from a hydroxy moiety, ether bond, ester bond and lactone ring, a phenylene group or naphthylene group,
X² is a single bond or *—C(═O)—O—,
* designates a point of attachment to the carbon atom in the backbone,
R¹¹ is halogen or a C₁-C₂₀ hydrocarbyl group which may contain a heteroatom, and
AL¹ and AL² are each independently an acid labile group.

In a preferred embodiment, the repeat unit having a carboxy group is represented by formula (b):

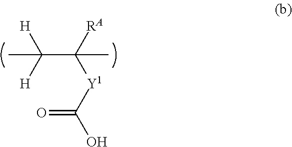

wherein R^{A} is hydrogen, fluorine, methyl or trifluoromethyl,
Y¹ is a single bond, phenylene group, naphthylene group or *—C(═O)—O—Y¹¹—, Y¹¹ is a C₁-C₁₀ saturated hydrocarbylene group which may contain at least one selected from a hydroxy moiety, ether bond, ester bond and lactone ring, a phenylene group or naphthylene group, and * designates a point of attachment to the carbon atom in the backbone.

In a preferred embodiment, the polymer further comprises at least one selected from repeat units having formulae (c1) and (c2):

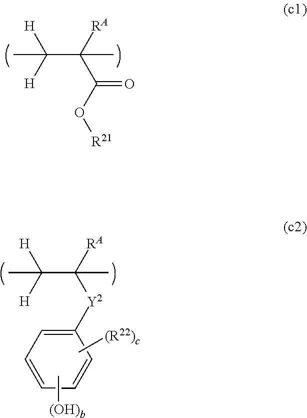

wherein b is 1 or 2, c is an integer of 0 to 4,
R^{A} is each independently hydrogen, fluorine, methyl or trifluoromethyl,
Y² is a single bond or *—C(═O)—O—, * designates a point of attachment to the carbon atom in the backbone,
R²¹ is a C₁-C₂₀ group containing at least one selected from hydroxy, cyano, carbonyl, ether bond, ester bond, sulfonic ester bond, carbonate bond, lactone ring, sultone ring, and carboxylic anhydride (—C(═O)—O—C(═O)—), and
R²² is halogen or a C₁-C₂₀ hydrocarbyl group which may contain a heteroatom.

In a preferred embodiment, the polymer further comprises repeat units having a photoacid generating group.

In a preferred embodiment, the resist composition may further comprise (D) a photoacid generator.

In a preferred embodiment, the resist composition further comprise (E) a quencher.

In a preferred embodiment, the resist composition further comprise (F) a surfactant.

In another aspect, the invention provides a pattern forming process comprising the steps of applying the resist composition of any one defined herein to a substrate to form a resist film thereon, exposing the resist film to KrF excimer laser, ArF excimer laser, electron beam or extreme ultraviolet and developing the exposed resist film in a developer.

### Advantageous Effects of the Invention

Since the resist composition of the invention exhibits a high contrast, a pattern profile with reduced LWR and high resolution can be constructed therefrom.

### DETAILED DESCRIPTION OF THE INVENTION

### [Resist Composition]

One embodiment of the invention is a resist composition comprising (A) a base polymer containing a polymer comprising repeat units having an acid labile group and repeat units having a carboxy group, (B) an organic solvent, and (C) a predetermined hypervalent iodine compound.

### (A) Base Polymer

A base polymer as component (A) contains a polymer comprising repeat units having an acid labile group. The repeat unit having an acid labile group is preferably a repeat unit having formula (a1) or a repeat unit having formula (a2). These repeat units are also referred to as repeat units (a1) and (a2).

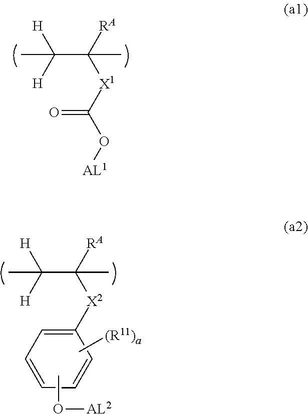

In formulae (a1) and (a2), “a” is an integer of 0 to 4, preferably 0 or 1. R^{A} is each independently hydrogen, fluorine, methyl or trifluoromethyl. X¹ is a single bond, phenylene group, naphthylene group or *—C(═O)—O—X¹¹—. X¹¹ is a C₁-C₁₀ saturated hydrocarbylene group which may contain at least one selected from a hydroxy moiety, ether bond, ester bond and lactone ring, a phenylene group or naphthylene group. X² is a single bond or *—C(═O)—O—. The asterisk (*) is a point of attachment to the carbon atom in the backbone. R¹¹ is halogen or a C₁-C₂₀ hydrocarbyl group which may contain a heteroatom. AL¹ and AL² are each independently an acid labile group.

The saturated hydrocarbylene group represented by X¹¹ may be straight, branched or cyclic. Examples thereof include C₁-C₁₀ alkanediyl groups such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, and decane-1,10-diyl; and C₃-C₁₀ cyclic saturated hydrocarbylene groups such as cyclopentanediyl, cyclohexanediyl, norbornanediyl and adamantanediyl.

Examples of the halogen atom represented by R¹¹ include fluorine, chlorine, bromine and iodine. The hydrocarbyl group represented by R¹¹ may be saturated or unsaturated and straight, branched or cyclic. Examples thereof include C₁-C₂₀ alkyl groups such as methyl, ethyl, n-propyl, isopropyl, n-butyl, isobutyl, sec-butyl, and tert-butyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, allyl, propenyl, butenyl, and hexenyl; C₃-C₂₀ cyclic unsaturated hydrocarbyl groups such as cyclohexenyl; C₆-C₂₀ aryl groups such as phenyl, naphthyl and thienyl; C₇-C₂₀ aralkyl groups such as benzyl, 1-phenylethyl and 2-phenylethyl; and combinations thereof. Inter alia, aryl groups are preferred. In the foregoing hydrocarbyl groups, some or all of the hydrogen atoms may be substituted by a moiety containing a heteroatom such as oxygen, sulfur, nitrogen or halogen, or some constituent —CH₂— may be substituted by a moiety containing a heteroatom such as oxygen, sulfur or nitrogen, so that the group may contain a hydroxy, fluorine, chlorine, bromine, iodine, cyano, carbonyl, ether bond, ester bond, sulfonic ester bond, carbonate bond, lactone ring, sultone ring, carboxylic anhydride (—C(═O)—O—C(═O)—) or haloalkyl moiety.

Examples of the structure of formula (a1) wherein X¹ is a variant are illustrated below, but not limited thereto. Herein R^{A} and AL¹ are as defined above.

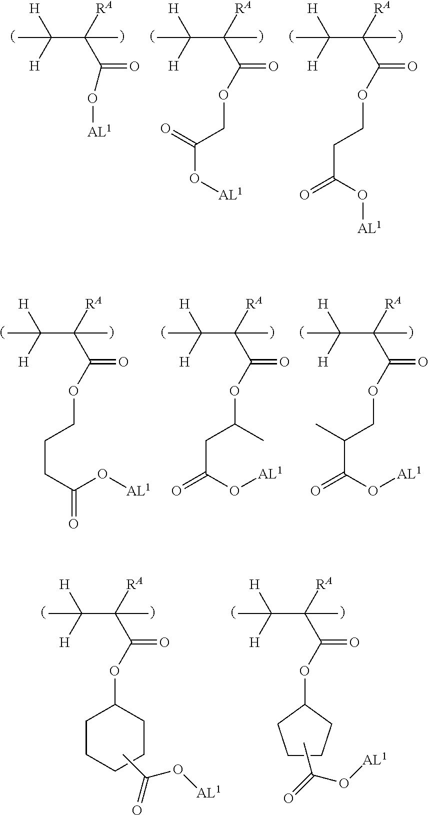

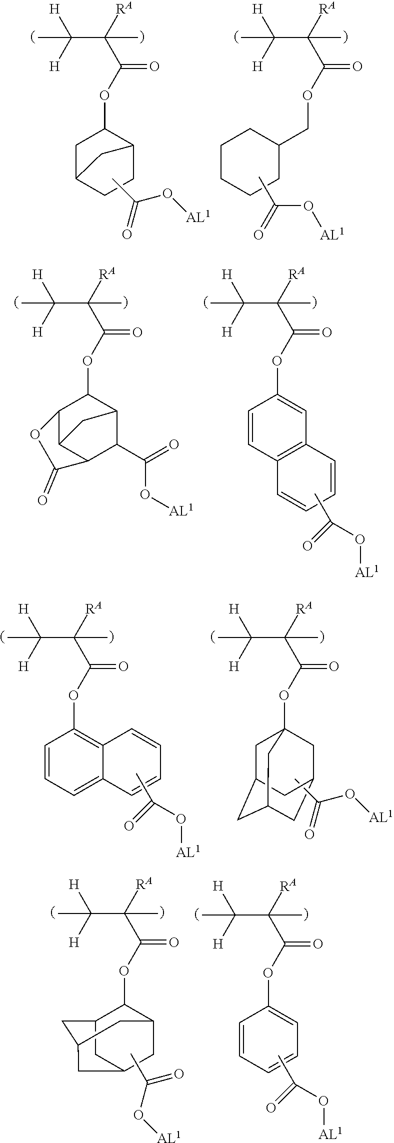

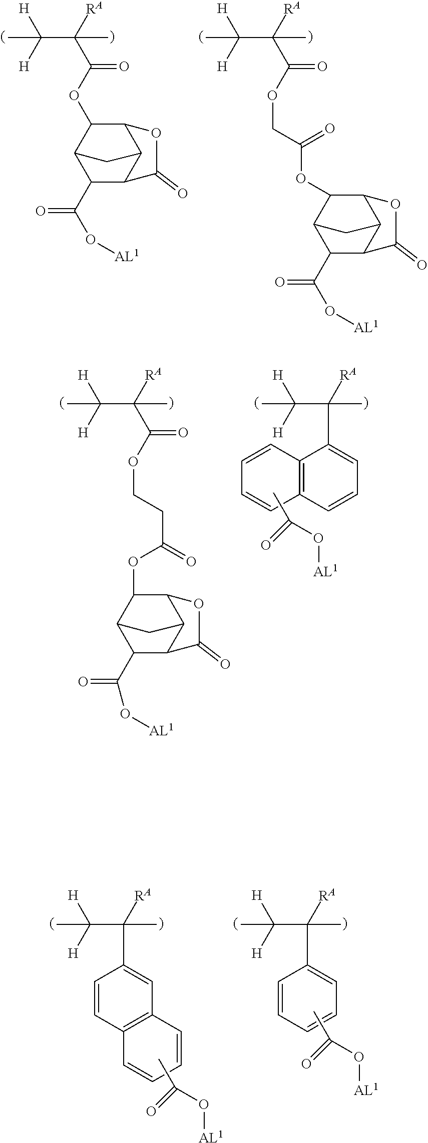

A polymer comprising repeat units (a1) turns alkali soluble through the mechanism that it is decomposed to generate a carboxy group under the action of acid.

The acid labile groups represented by AL¹ and AL² may be selected from a variety of such groups. Preferred examples of the acid labile group are groups of any of the following formulae (L1) to (L4), C₄-C₂₀, preferably C₄-C₁₅ tertiary hydrocarbyl groups, trihydrocarbylsilyl groups in which each hydrocarbyl moiety has 1 to 6 carbon atoms, and C₄-C₂₀ hydrocarbyl groups containing a carbonyl moiety, ether bond or ester bond.

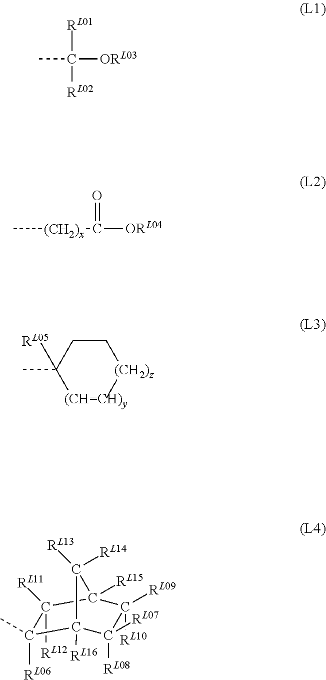

Herein the broken line is a bond.

In formula (L1), R^{L01} and R^{L02} are each independently hydrogen or a C₁-C₁₈, preferably C₁-C₁₀ saturated hydrocarbyl group. The saturated hydrocarbyl group may be straight, branched or cyclic and examples thereof include alkyl groups such as methyl, ethyl, n-propyl, isopropyl, n-butyl, sec-butyl, tert-butyl, n-octyl, and 2-ethylhexyl; and cyclic saturated hydrocarbyl groups such as cyclopentyl, cyclohexyl, norbornyl, tricyclodecanyl, tetracyclododecanyl, and adamantyl.

In formula (L1), R^{L03} is a C₁-C₁₈, preferably C₁-C₁₀ hydrocarbyl group which may contain a heteroatom. The hydrocarbyl group may be saturated or unsaturated and straight, branched or cyclic. Saturated hydrocarbyl groups are preferred. In the saturated hydrocarbyl group, some or all of the hydrogen atoms may be substituted by hydroxy, saturated hydrocarbyloxy, oxo, amino, saturated hydrocarbylamino or the like, or some constituent —CH₂— may be substituted by a moiety containing a heteroatom, typically oxygen. Suitable saturated hydrocarbyl groups are as exemplified above for the saturated hydrocarbyl groups R^{L01} and R^{L02}. Examples of the substituted saturated hydrocarbyl group are shown below.

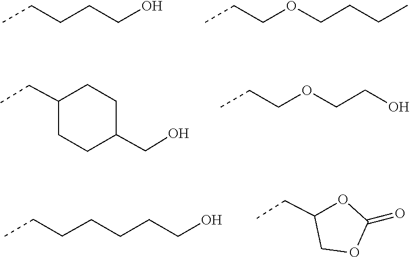

Herein the broken line is a bond.

Any two of R^{L01}, R^{L02}, and R^{L03} may bond together to form a ring with the carbon atom or the carbon and oxygen atoms to which they are attached. When any two of R^{L01}, R^{L02} and R^{L03} form a ring, each is independently a C₁-C₁₈, preferably C₁-C₁₀ alkanediyl group.

In formula (L2), R^{L04} is a C₄-C₂₀, preferably C₄-C₁₅ tertiary hydrocarbyl group, a trialkylsilyl group in which each alkyl moiety has 1 to 6 carbon atoms, a C₄-C₂₀ saturated hydrocarbyl group containing a carbonyl moiety, ether bond or ester bond, or a group of formula (L1). The subscript x is an integer of 0 to 6.

Of the groups R^{L04}, the tertiary hydrocarbyl group may be branched or cyclic, and examples thereof include tert-butyl, tert-pentyl, 1,1-diethylpropyl, 2-cyclopentylpropan-2-yl, 2-cyclohexylpropan-2-yl, 2-(bicyclo[2.2.1]heptan-2-yl)propan-2-yl, 2-(adamantan-1-yl)propan-2-yl, 1-ethylcyclopentyl, 1-butylcyclopentyl, 1-ethylcyclohexyl, 1-butylcyclohexyl, 1-ethyl-2-cyclopentenyl, 1-ethyl-2-cyclohexenyl, 2-methyl-2-adamantyl, and 2-ethyl-2-adamantyl. Exemplary trialkylsilyl groups include trimethylsilyl, triethylsilyl, and dimethyl-tert-butylsilyl. Exemplary saturated hydrocarbyl groups containing a carbonyl, ether bond or ester bond include 3-oxocyclohexyl, 4-methyl-2-oxooxan-4-yl, and 5-methyl-2-oxooxolan-5-yl.

In formula (L3), R^{L05} is a C₁-C₈ saturated hydrocarbyl group or C₆-C₂₀ aryl group which may be substituted. The saturated hydrocarbyl group which may be substituted may be straight, branched or cyclic and examples thereof include alkyl groups such as methyl, ethyl, n-propyl, isopropyl, n-butyl, sec-butyl, tert-butyl, n-pentyl, tert-pentyl, and n-hexyl; cyclic saturated hydrocarbyl groups such as cyclopentyl and cyclohexyl; and substituted forms of the foregoing in which some or all of the hydrogen atoms are substituted by hydroxy, C₁-C₈ saturated hydrocarbyloxy, carboxy, C₁-C₈ saturated hydrocarbylcarbonyl, oxo, amino, C₁-C₈ saturated hydrocarbylamino, cyano, mercapto, C₁-C₈ saturated hydrocarbylthio, sulfo or the like. Examples of the aryl group which may be substituted include phenyl, methylphenyl, naphthyl, anthryl, phenanthryl, and pyrenyl, and substituted forms of the foregoing in which some or all of the hydrogen atoms are substituted by hydroxy, C₁-C₈ saturated hydrocarbyloxy, carboxy, C₁-C₈ saturated hydrocarbylcarbonyl, oxo, amino, C₁-C₈ saturated hydrocarbylamino, cyano, mercapto, C₁-C₈ saturated hydrocarbylthio, sulfo or the like.

In formula (L3), y is equal to 0 or 1, z is an integer of 0 to 3, and 2y+z is equal to 2 or 3.

In formula (L4), R^{L06} is a C₁-C₈ saturated hydrocarbyl group or C₆-C₂₀ aryl group which may be substituted. Examples of the saturated hydrocarbyl and aryl groups which may be substituted are the same as exemplified above for R^{L05}.

In formula (L4), R^{L07} to R^{L16} are each independently hydrogen or a C₁-C₁₅ hydrocarbyl group which may be substituted. The hydrocarbyl group may be saturated or unsaturated and straight, branched or cyclic. Saturated hydrocarbyl groups are preferred. Examples of the hydrocarbyl group include alkyl groups such as methyl, ethyl, n-propyl, isopropyl, n-butyl, sec-butyl, tert-butyl, tert-pentyl, n-pentyl, n-hexyl, n-octyl, n-nonyl, and n-decyl; cyclic saturated hydrocarbyl groups such as cyclopentyl, cyclohexyl, cyclopentylmethyl, cyclopentylethyl, cyclopentylbutyl, cyclohexylmethyl, cyclohexylethyl and cyclohexylbutyl; and substituted forms of the foregoing in which some or all of the hydrogen atoms are substituted by hydroxy, C₁-C₈ saturated hydrocarbyloxy, carboxy, C₁-C₈ saturated hydrocarbyloxycarbonyl, oxo, amino, C₁-C₈ saturated hydrocarbylamino, cyano, mercapto, C₁-C₈ saturated hydrocarbylthio, sulfo or the like. Alternatively, two of R^{L07} to R^{L16} may bond together to form a ring with the carbon atom to which they are attached (for example, a pair of R^{L07} and R^{L08}, R^{L07} and R^{L09}, R^{L07} and R^{L10}, R^{L08} and R^{L10}, R^{L09} and R^{L10}, R^{L11} and R^{L12}, R^{L13} and R^{L14}, or a similar pair form a ring). Each of ring-forming R^{L07} to R^{L16} represents a C₁-C₁₅ hydrocarbylene group, examples of which are the ones exemplified above for the hydrocarbyl groups, with one hydrogen atom being eliminated. Two of R^{L07} to R^{L16} which are attached to vicinal carbon atoms may bond together directly to form a double bond (for example, a pair of R^{L07} and R^{L09}, R^{L09} and R^{L15}, R^{L13} and R^{L15}, R^{L14} and R^{L15}, or a similar pair).

Of the acid labile groups having formula (L1), the straight and branched ones are exemplified by the following groups, but not limited thereto.

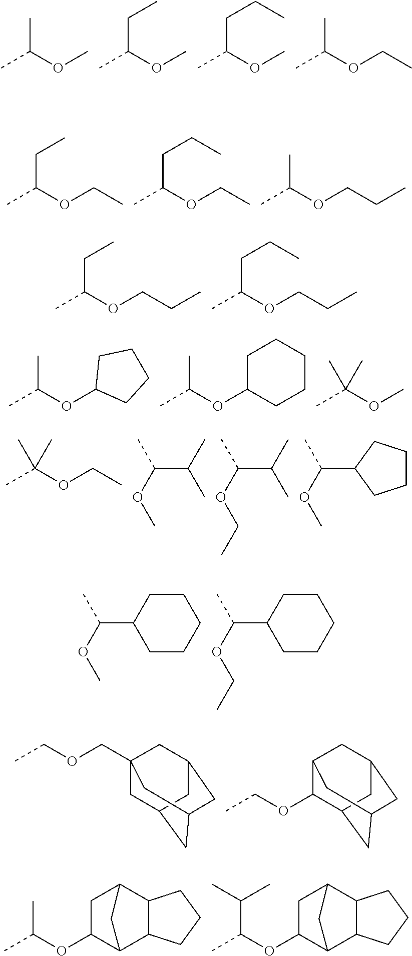

Herein the broken line is a bond.

Of the acid labile groups having formula (L1), the cyclic ones are, for example, tetrahydrofuran-2-yl, 2-methyltetrahydrofuran-2-yl, tetrahydropyran-2-yl, and 2-methyltetrahydropyran-2-yl.

Examples of the acid labile group having formula (L2) include tert-butoxycarbonyl, tert-butoxycarbonylmethyl, tert-pentyloxycarbonyl, tert-pentyloxycarbonylmethyl, 1,1-diethylpropyloxycarbonyl, 1,1-diethylpropyloxycarbonylmethyl, 1-ethylcyclopentyloxycarbonyl, 1-ethylcyclopentyloxycarbonylmethyl, 1-ethyl-2-cyclopentenyloxycarbonyl, 1-ethyl-2-cyclopentenyloxycarbonylmethyl, 1-ethoxyethoxycarbonylmethyl, 2-tetrahydropyranyloxycarbonylmethyl, and 2-tetrahydrofuranyloxycarbonylmethyl groups.

Examples of the acid labile group having formula (L3) include 1-methylcyclopentyl, 1-ethylcyclopentyl, 1-n-propylcyclopentyl, 1-isopropylcyclopentyl, 1-n-butylcyclopentyl, 1-sec-butylcyclopentyl, 1-cyclohexylcyclopentyl, 1-(4-methoxy-n-butyl)cyclopentyl, 1-methylcyclohexyl, 1-ethylcyclohexyl, 3-methyl-1-cyclopenten-3-yl, 3-ethyl-1-cyclopenten-3-yl, 3-methyl-1-cyclohexen-3-yl, and 3-ethyl-1-cyclohexen-3-yl groups.

Of the acid labile groups having formula (L4), groups having the following formulae (L4-1) to (L4-4) are preferred.

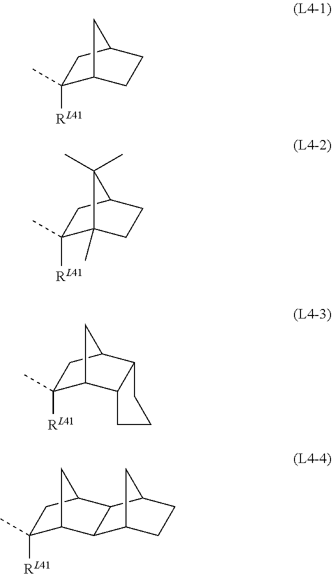

In formulae (L4-1) to (L4-4), the broken line denotes a bonding site and direction. R^{L41} is each independently a C₁-C₁₀ hydrocarbyl group. The hydrocarbyl group may be saturated or unsaturated and straight, branched or cyclic. Saturated hydrocarbyl groups are preferred. Suitable hydrocarbyl groups include alkyl groups such as methyl, ethyl, n-propyl, isopropyl, n-butyl, sec-butyl, tert-butyl, n-pentyl, tert-pentyl, and n-hexyl; and cyclic saturated hydrocarbyl groups such as cyclopentyl and cyclohexyl.

For formulae (L4-1) to (L4-4), there can exist stereoisomers (enantiomers or diastereomers). Each of formulae (L4-1) to (L4-4) collectively represents all such stereoisomers. When the acid labile group is of formula (L4), there may be contained a plurality of stereoisomers.

For example, formula (L4-3) represents one or a mixture of two selected from groups having the following formulae (L4-3-1) and (L4-3-2).

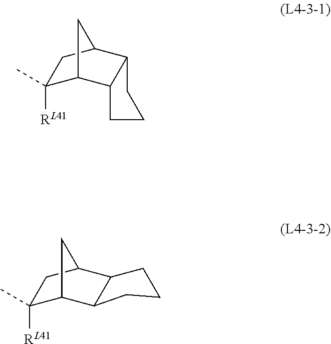

Herein R^{L41} is as defined above, and the broken line denotes a bonding site and direction.

Similarly, formula (L4-4) represents one or a mixture of two or more selected from groups having the following formulae (L4-4-1) to (L4-4-4).

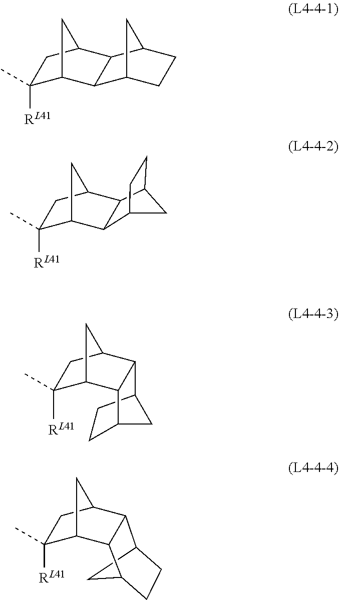

Herein R^{L41} is as defined above, and the broken line denotes a bonding site and direction.

Each of formulae (L4-1) to (L4-4), (L4-3-1), (L4-3-2), and (L4-4-1) to (L4-4-4) collectively represents an enantiomer thereof and a mixture of enantiomers.

It is noted that in the above formulae (L4-1) to (L4-4), (L4-3-1), (L4-3-2), and (L4-4-1) to (L4-4-4), the bond direction is on the exo side relative to the bicyclo[2.2.1]heptane ring, which ensures high reactivity for acid catalyzed elimination reaction (see JP-A 2000-336121). In preparing these monomers having a tertiary exo-saturated hydrocarbyl group of bicyclo[2.2.1]heptane skeleton as a substituent group, there may be contained monomers substituted with an endo-alkyl group as represented by the following formulae (L4-1-endo) to (L4-4-endo). For good reactivity, an exo proportion of at least 50 mol % is preferred, with an exo proportion of at least 80 mol % being more preferred.

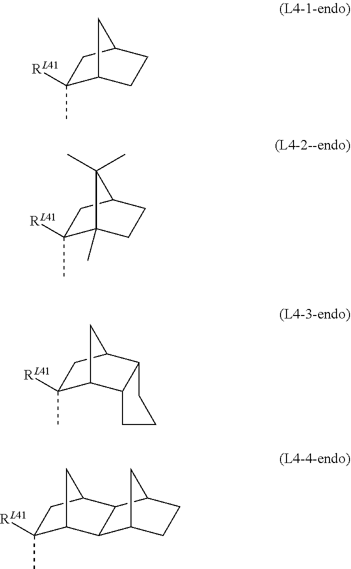

Herein R^{L41} is as defined above, and the broken line denotes a bonding site and direction.

Examples of the acid labile group having formula (L4) are given below, but not limited thereto.

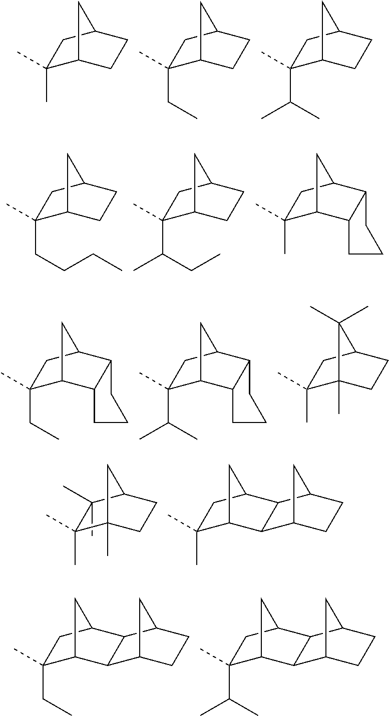

Herein the broken line is a bond.

Of the acid labile groups represented by AL¹ and AL², examples of the C₄-C₂₀ tertiary hydrocarbyl groups, trihydrocarbylsilyl groups in which each hydrocarbyl moiety has 1 to 6 carbon atoms, and C₄-C₂₀ saturated hydrocarbyl groups containing carbonyl, ether bond or ester bond are as exemplified above for R^{L04}.

Illustrative examples of the repeat unit (a1) are given below, but not limited thereto. Herein R^{A} is as defined above.

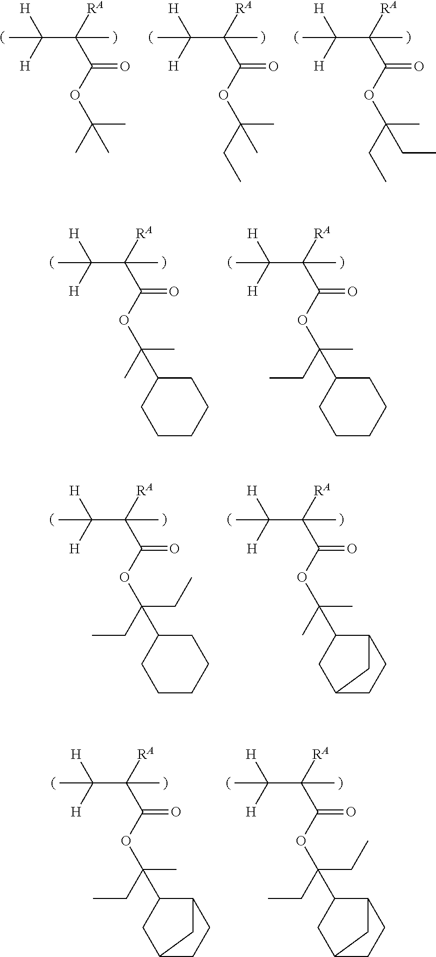

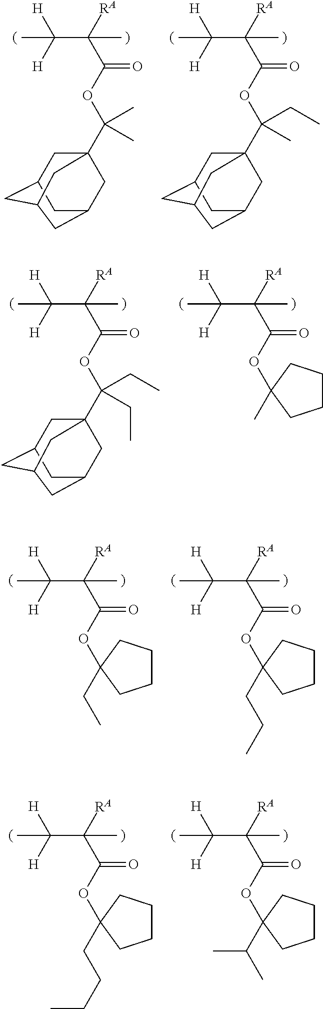

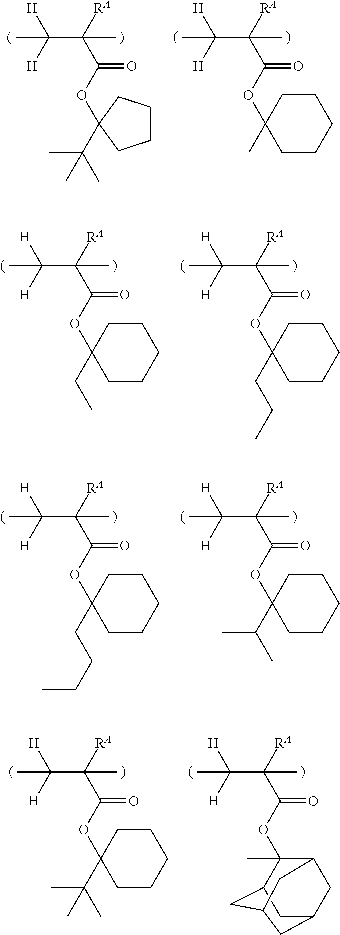

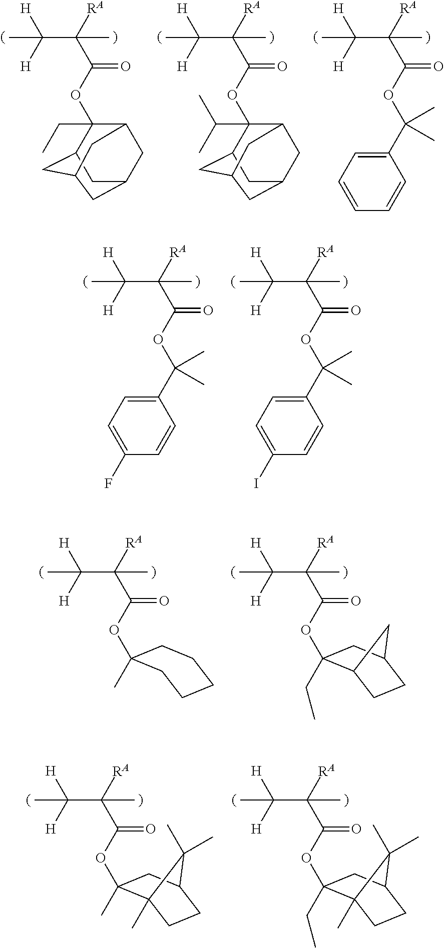

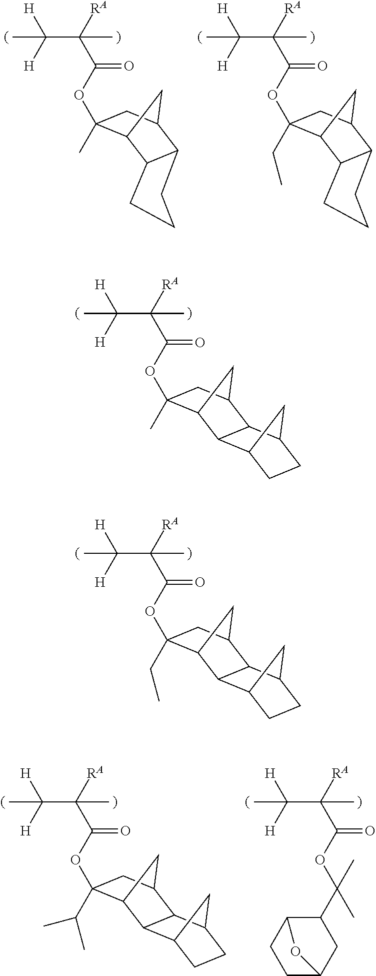

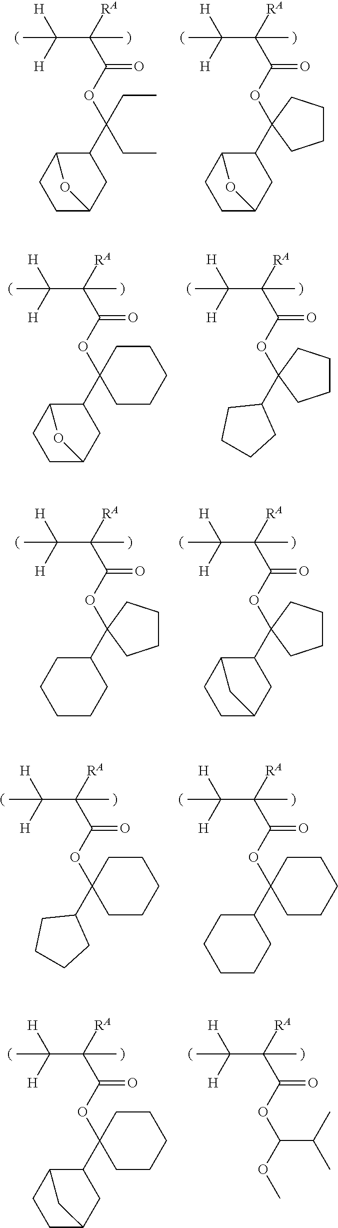

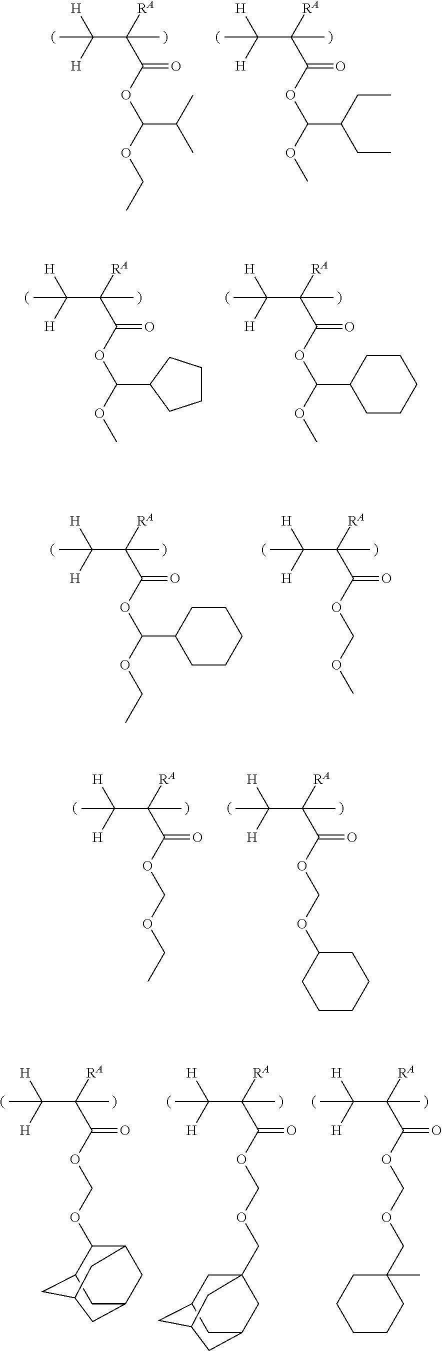

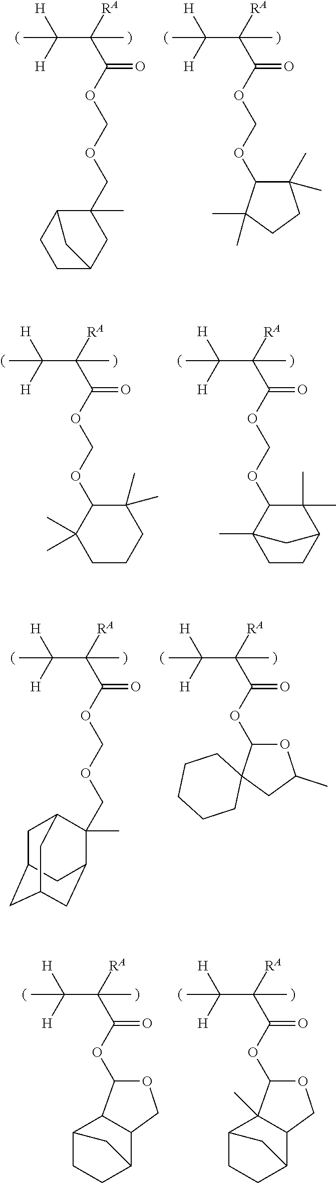

While the foregoing examples correspond to those units wherein X¹ is a single bond, X¹ which is other than a single bond may be combined with similar acid labile groups. Examples of units wherein X¹ is other than a single bond are substantially the same as illustrated above.

Like the repeat units (a1), a polymer comprising repeat units (a2) turns alkali soluble through the mechanism that it is decomposed to generate a hydroxy group under the action of acid. Illustrative examples of the repeat units (a2) are given below, but not limited thereto. Herein R^{A} is as defined above.

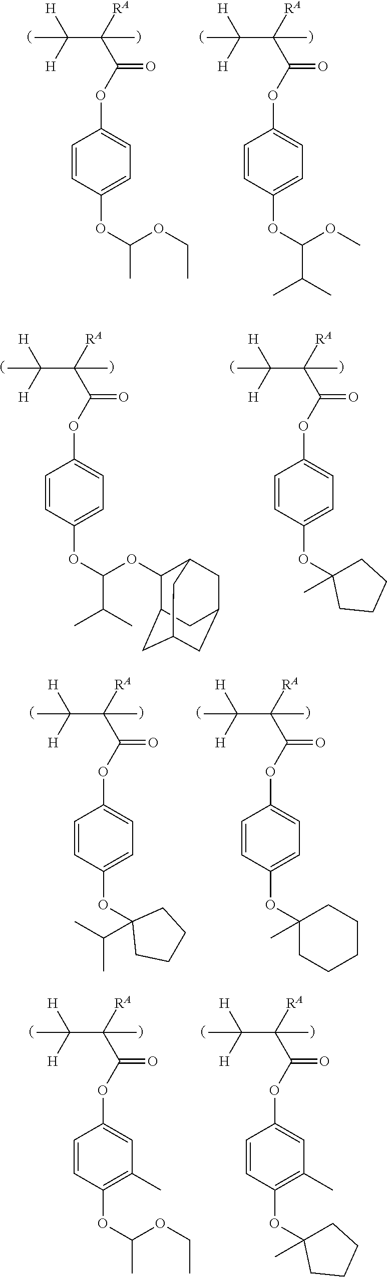

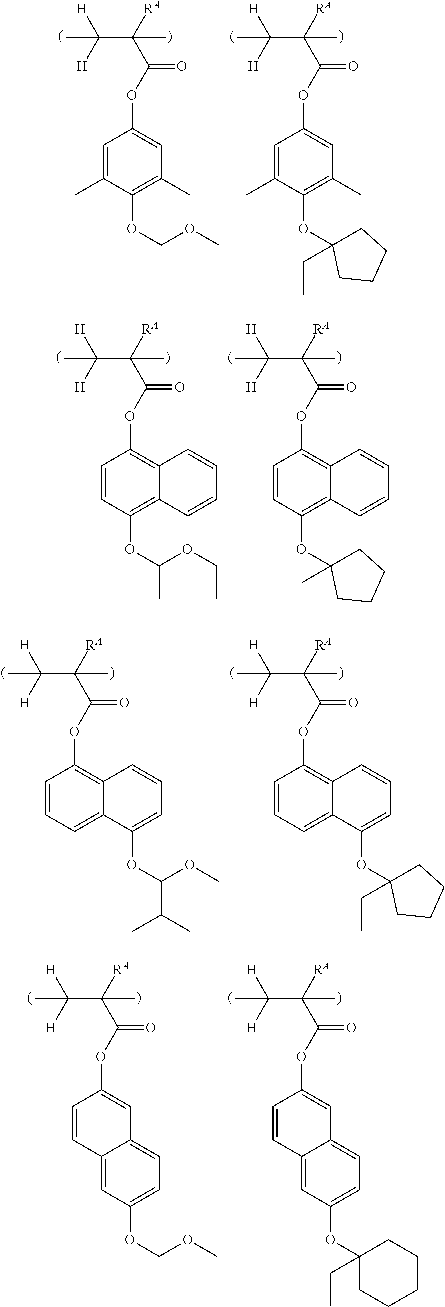

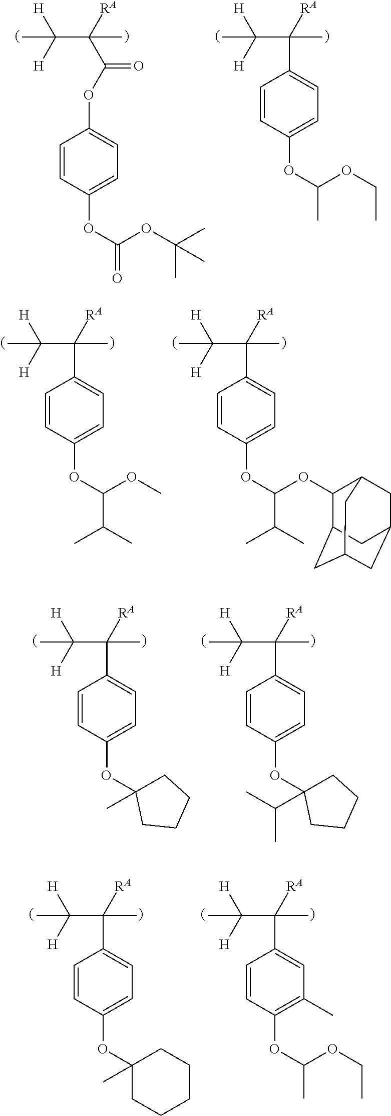

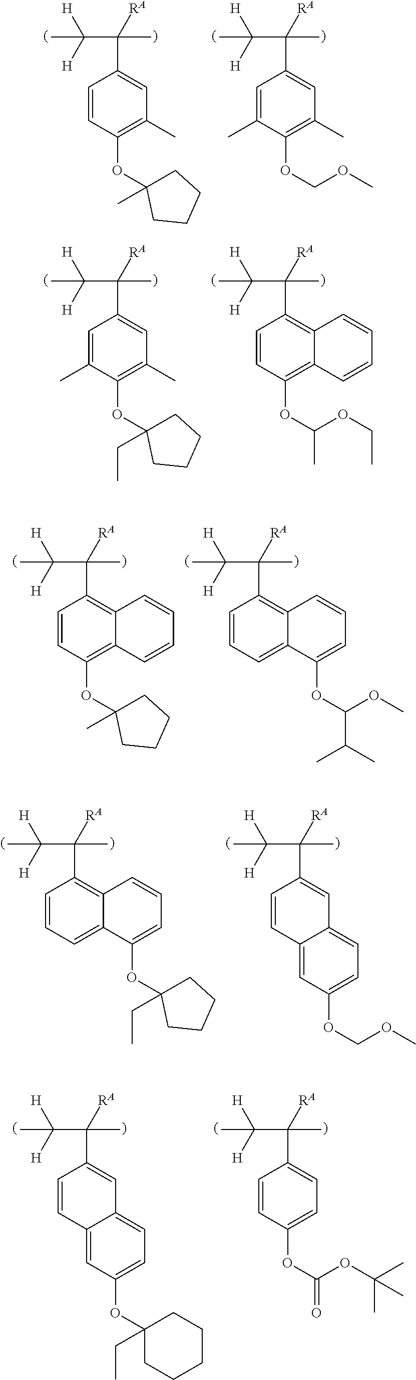

In addition to the repeat units having an acid labile group, the polymer is characterized by comprising repeat units having a carboxy group, which are also referred to as repeat units (b). The repeat unit (b) is preferably a repeat unit having formula (b).

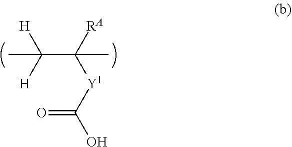

In formula (b), R^{A} is hydrogen, fluorine, methyl or trifluoromethyl. Y¹ is a single bond, phenylene group, naphthylene group or *—C(═O)—O—Y¹¹—. Y¹¹ is a C₁-C₁₀ saturated hydrocarbylene group which may contain at least one selected from a hydroxy moiety, ether bond, ester bond and lactone ring, a phenylene group or naphthylene group. The asterisk (*) designates a point of attachment to the carbon atom in the backbone. The saturated hydrocarbylene group represented by Y¹ may be straight, branched or cyclic. Examples thereof are as exemplified above for the saturated hydrocarbylene group X¹¹ in formula (a1).

Illustrative examples of the repeat units (b) are given below, but not limited thereto. Herein R^{A} is as defined above.

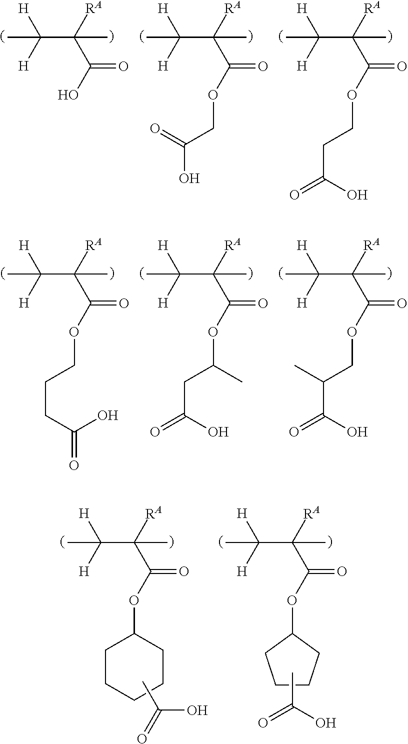

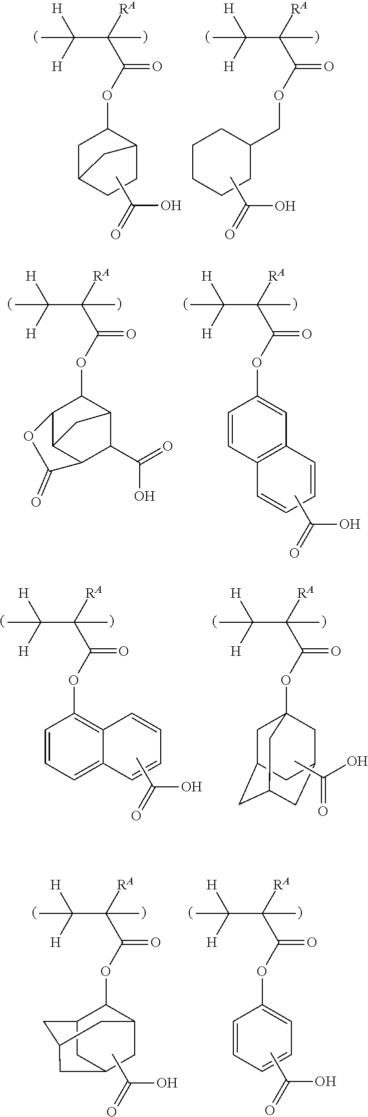

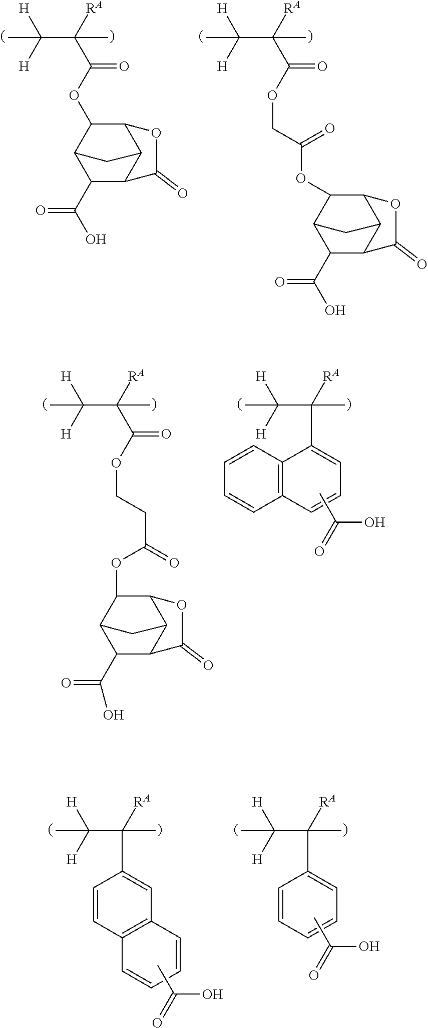

In a preferred embodiment, the polymer further comprises at least one selected from repeat units having formula (c1) and repeat units having formula (c2), which are also referred to as repeat units (c1) and (c2).

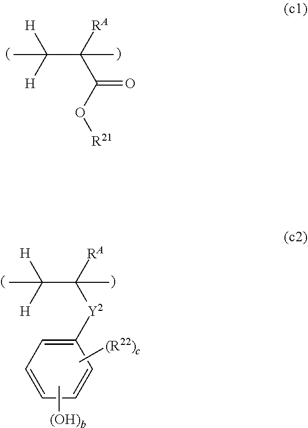

In formulae (c1) and (c2), b is 1 or 2. c is an integer of 0 to 4, preferably 0 or 1. R^{A} is each independently hydrogen, fluorine, methyl or trifluoromethyl. Y² is a single bond or *—C(═O)—O—. The asterisk (*) designates a point of attachment to the carbon atom in the backbone. R²¹ is a C₁-C₂₀ group containing at least one selected from hydroxy, cyano, carbonyl, ether bond, ester bond, sulfonic ester bond, carbonate bond, lactone ring, sultone ring, and carboxylic anhydride (—C(═O)—O—C(═O)—). R²² is halogen or a C₁-C₂₀ hydrocarbyl group which may contain a heteroatom.

Examples of the halogen atom represented by R²² are as exemplified above for the halogen atom R¹¹. The hydrocarbyl group represented by R²² may be saturated or unsaturated and straight, branched or cyclic. Examples thereof are as exemplified above for the hydrocarbyl group R¹¹.

Illustrative examples of the repeat units (c1) are given below, but not limited thereto. Herein R^{A} is as defined above.

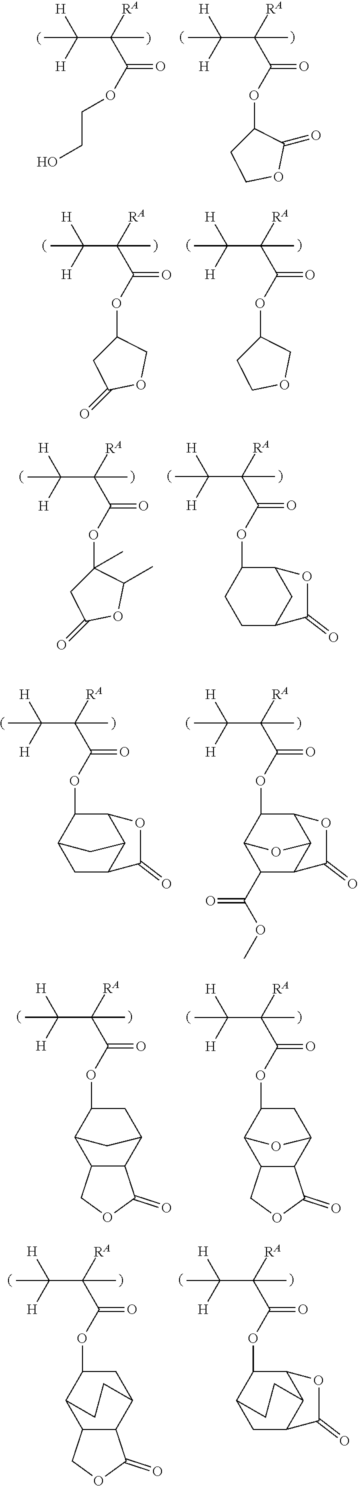

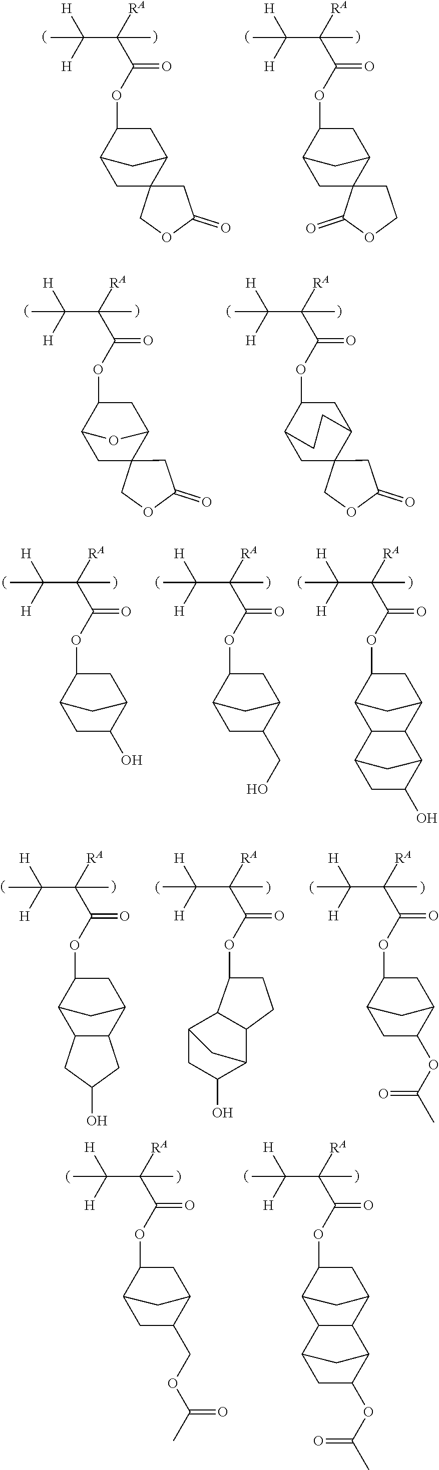

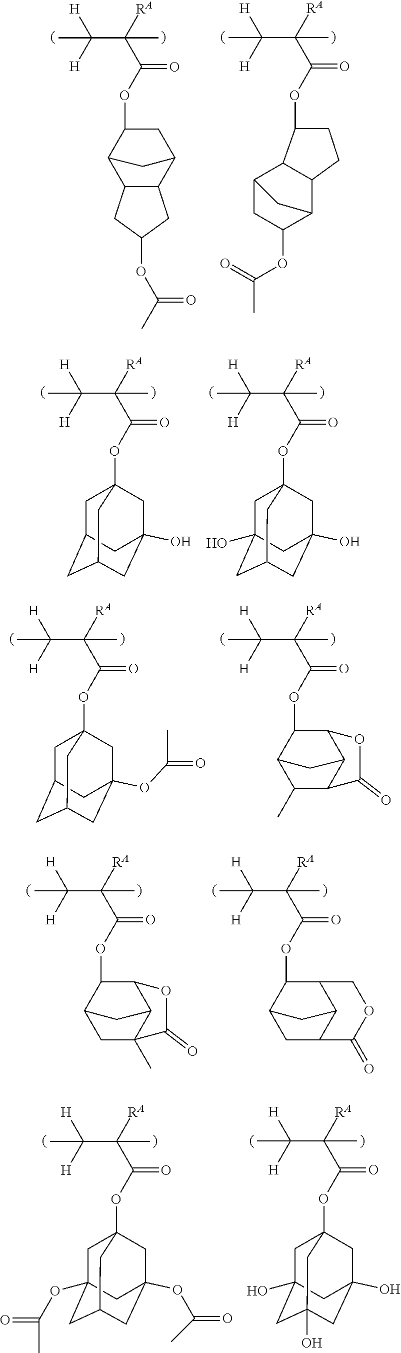

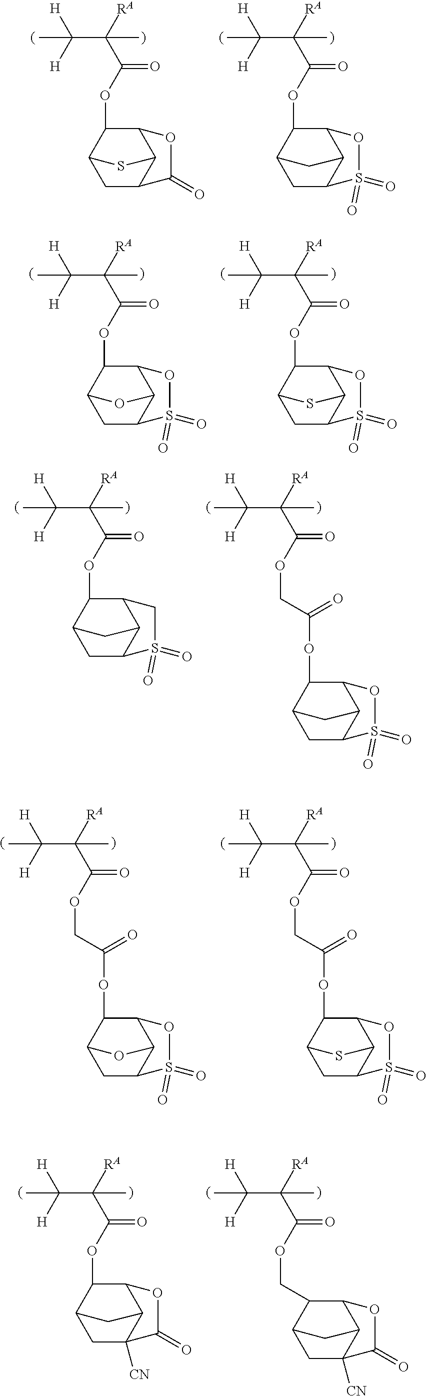

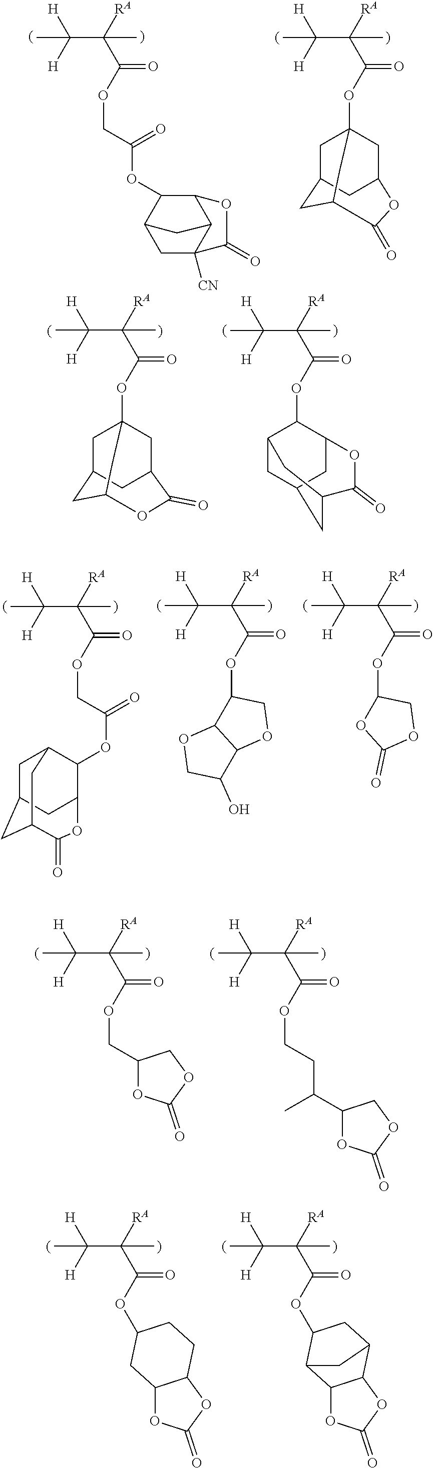

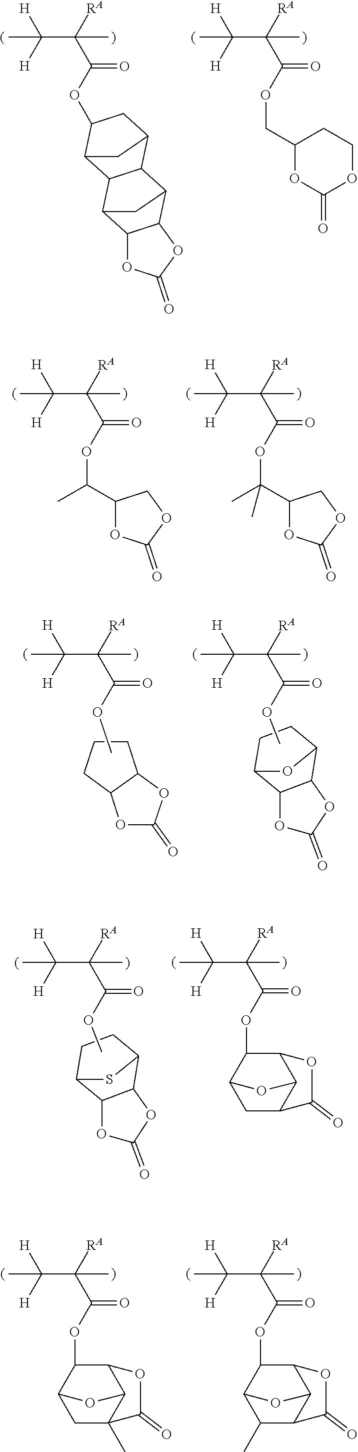

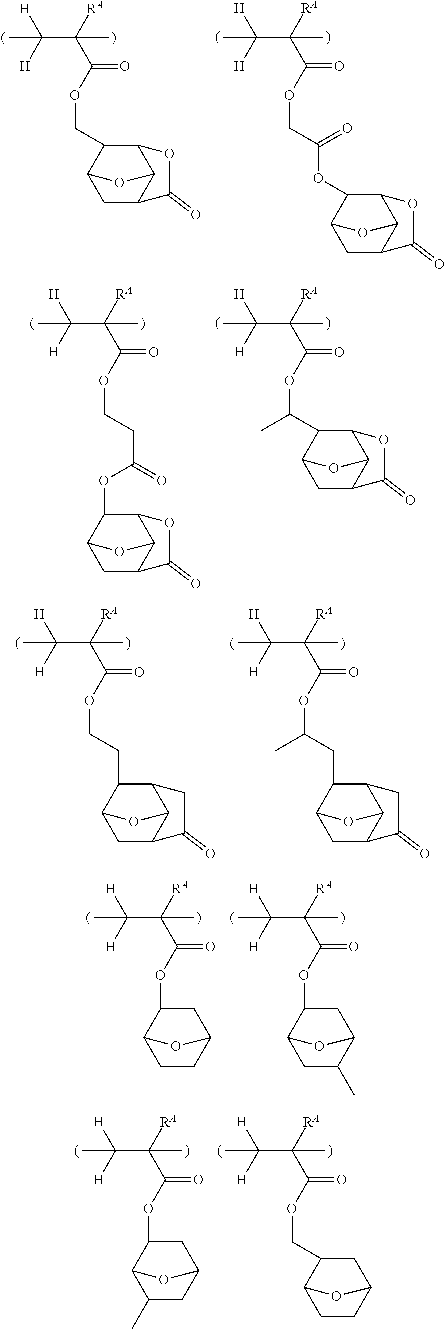

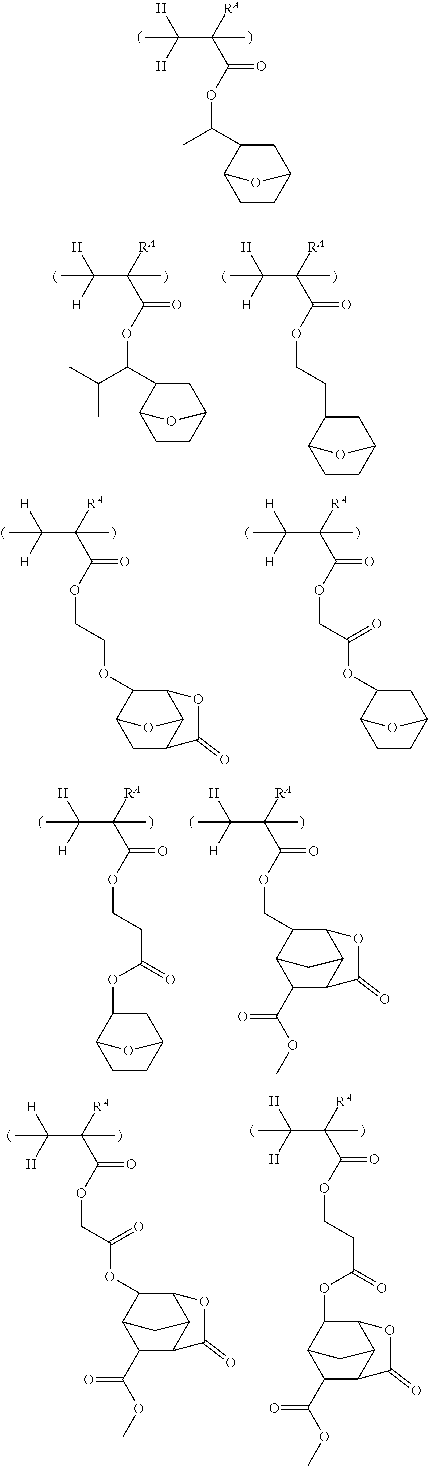

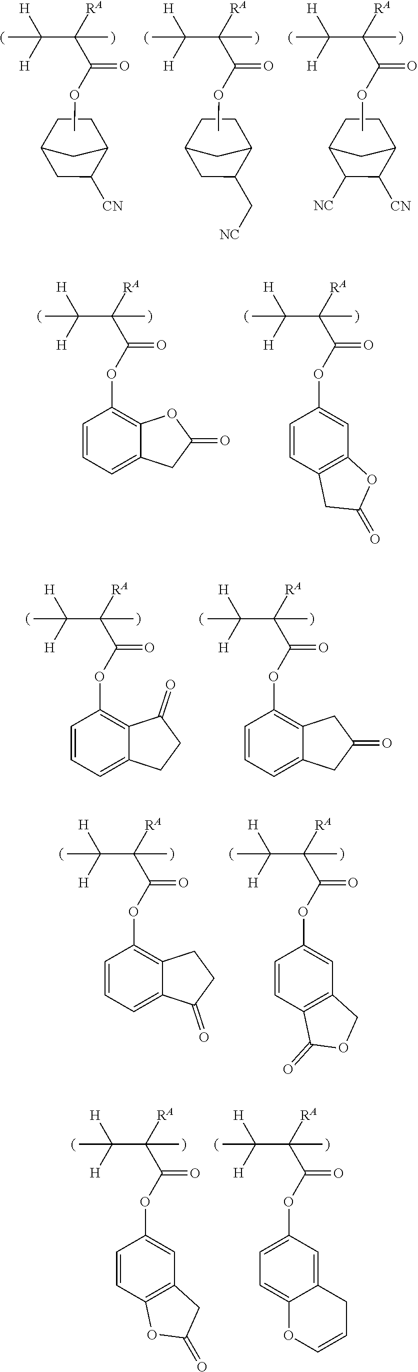

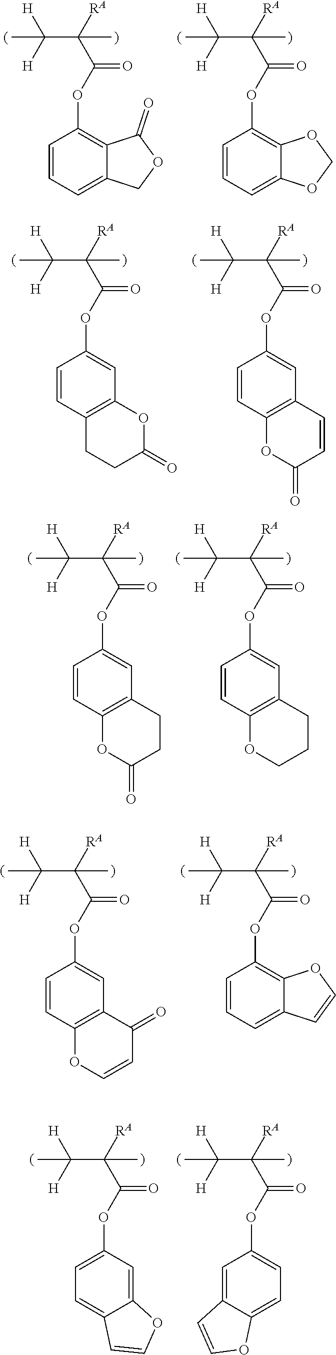

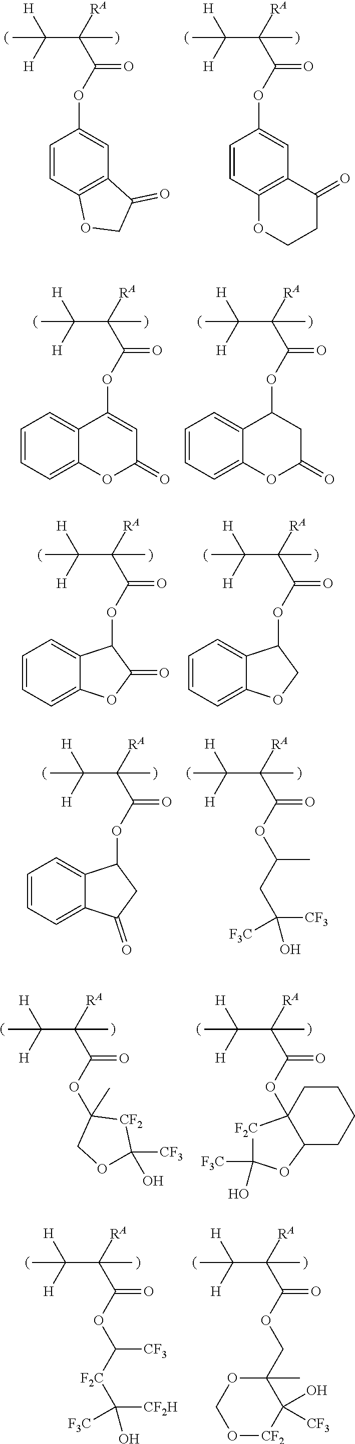

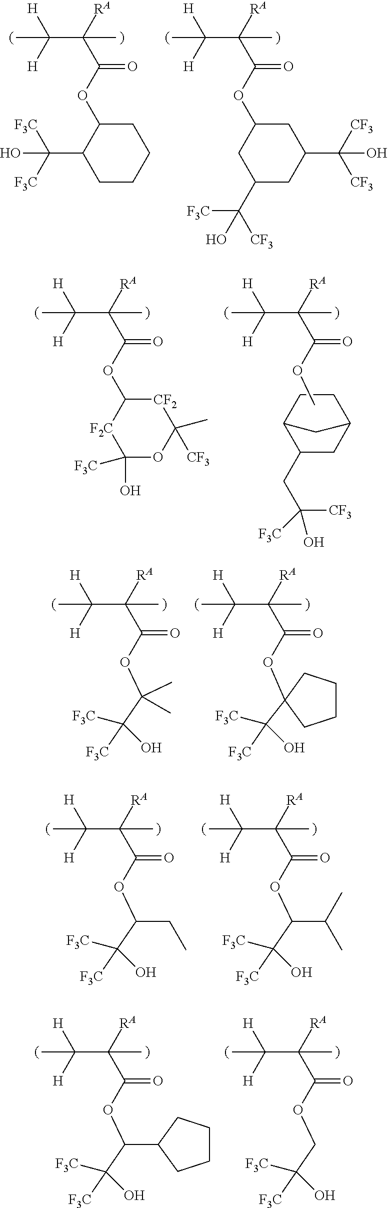

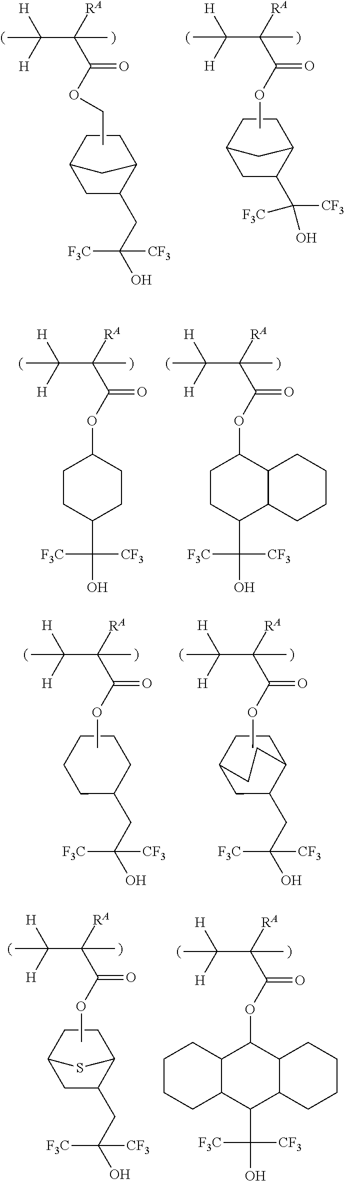

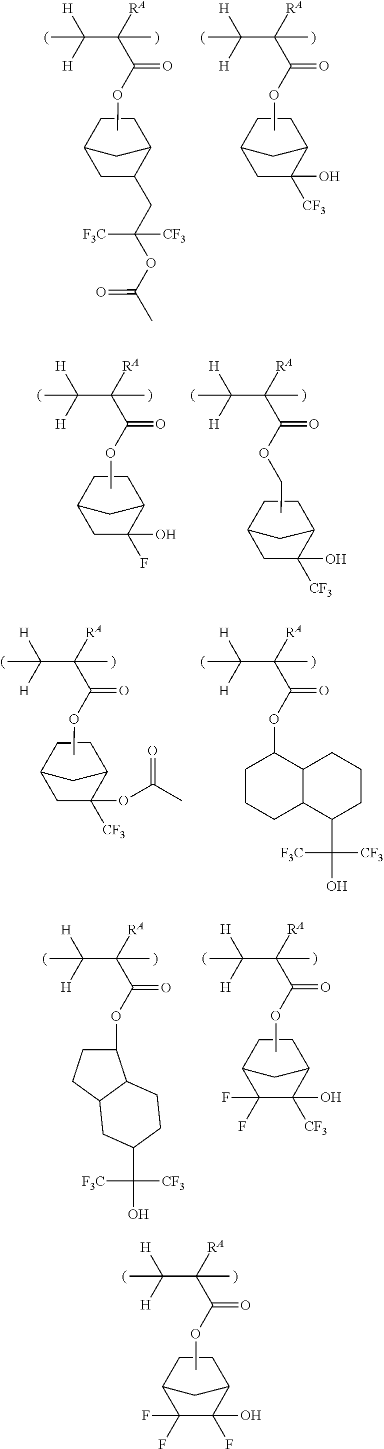

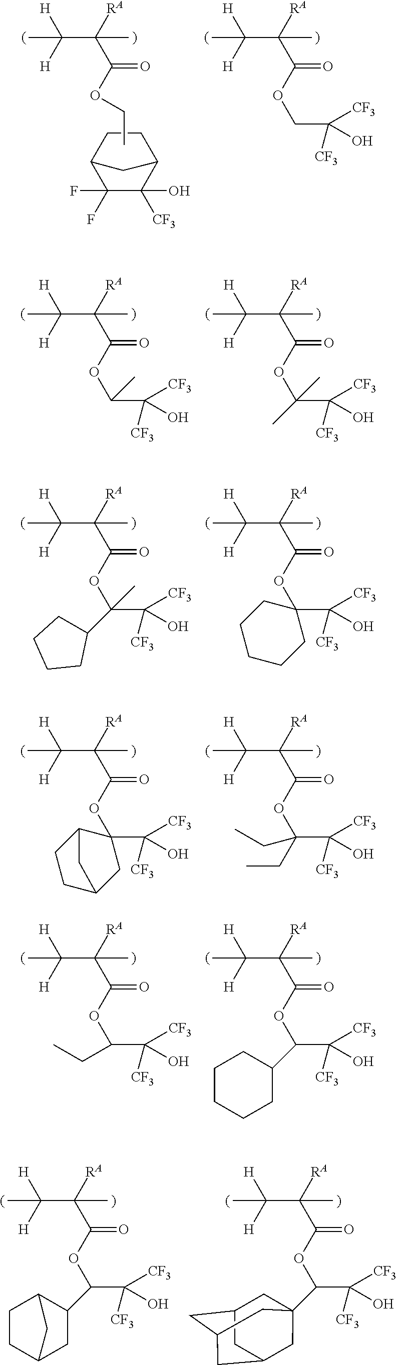

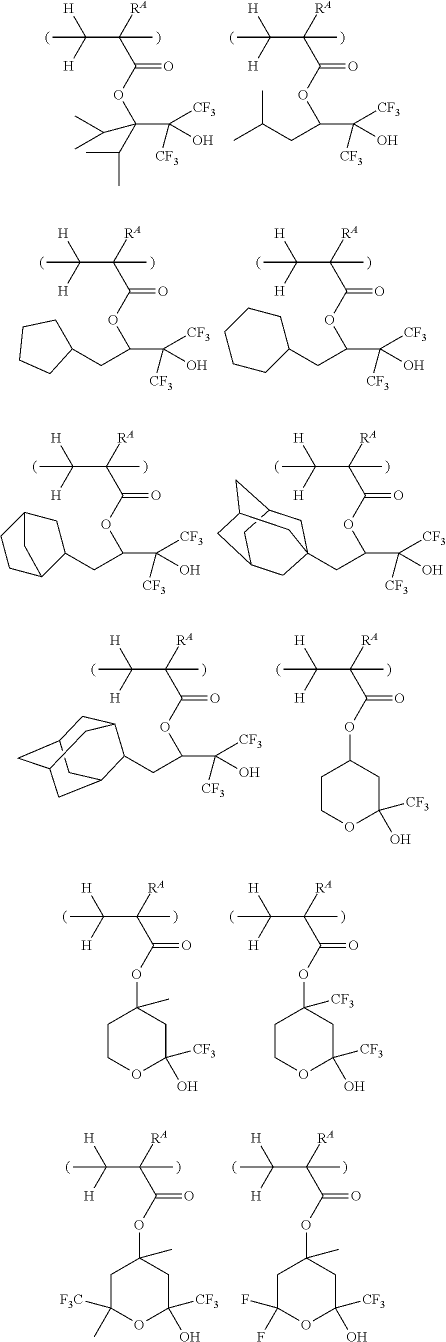

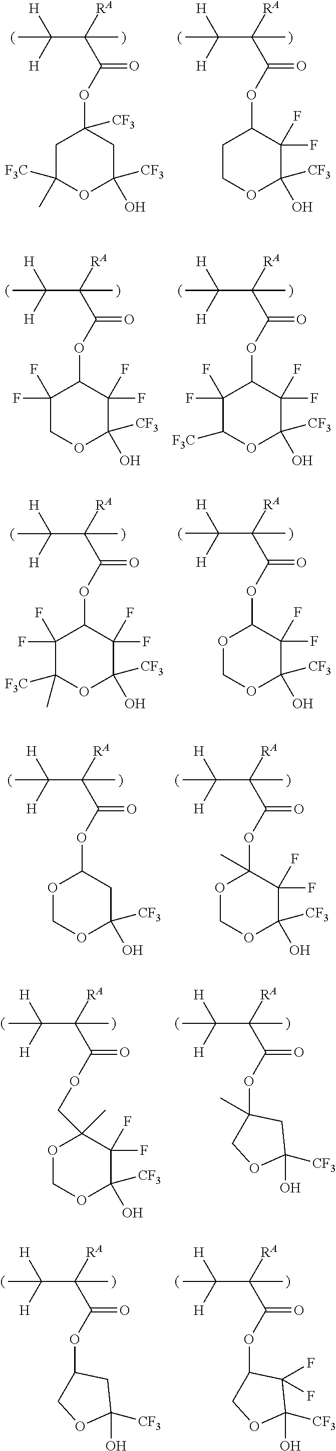

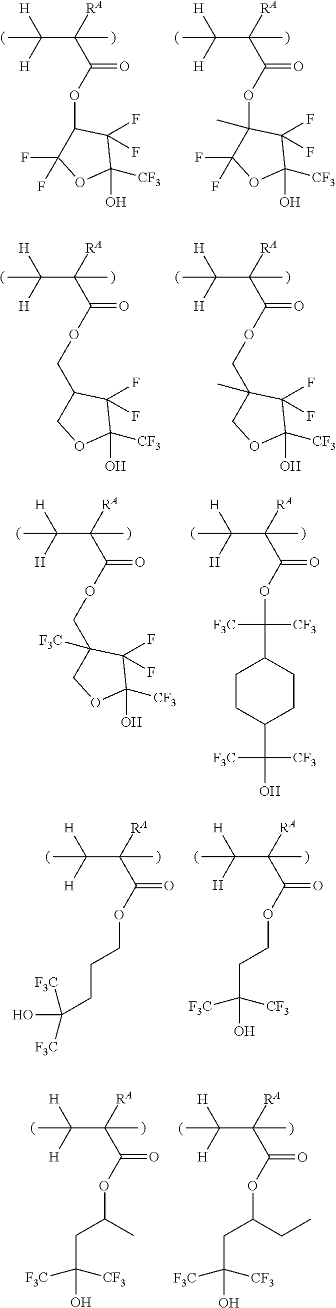

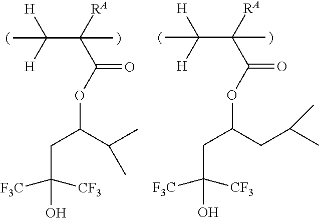

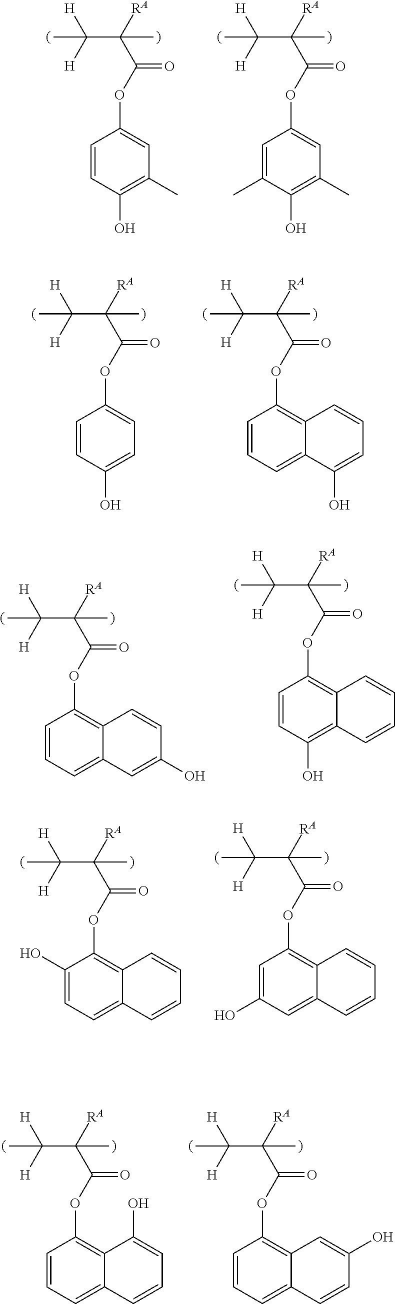

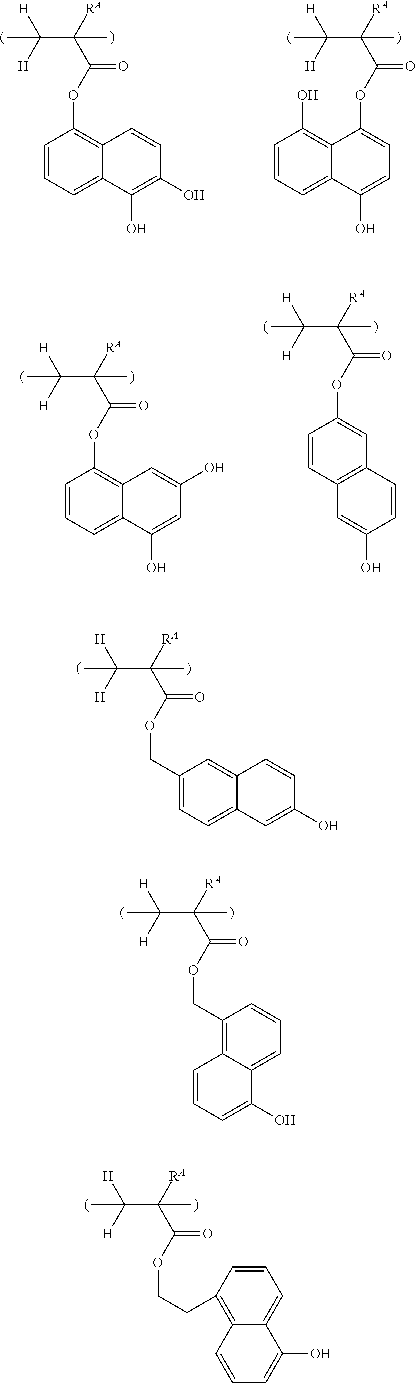

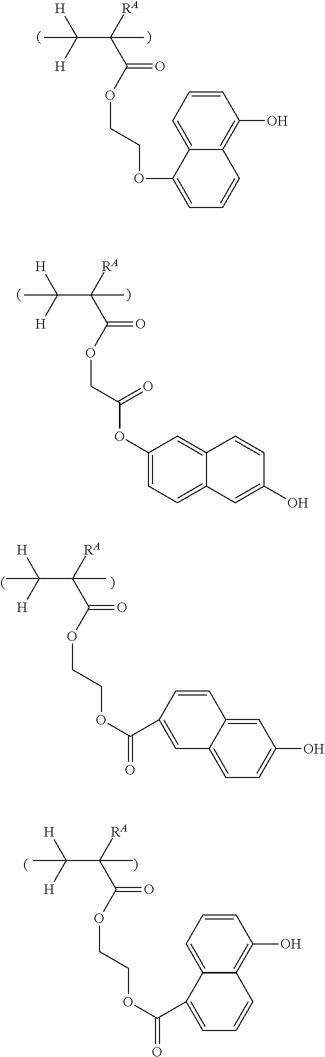

Illustrative examples of the repeat units (c2) are given below, but not limited thereto. Herein R^{A} is as defined above.

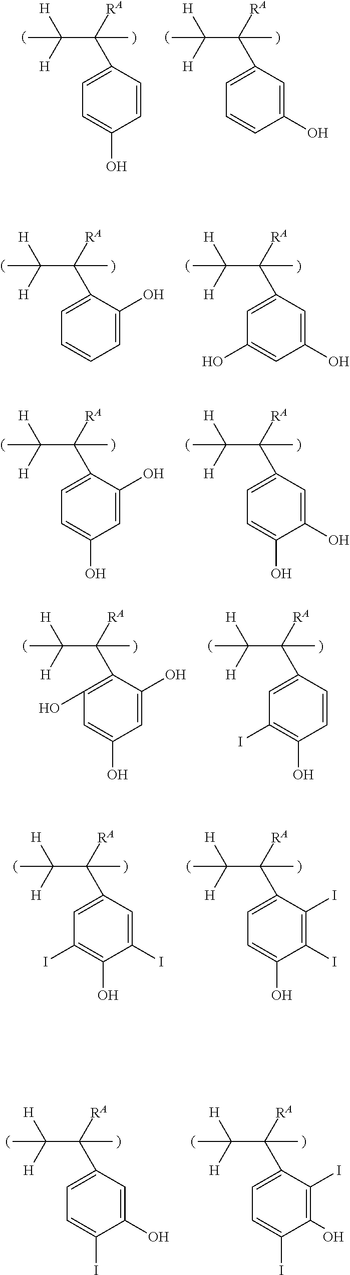

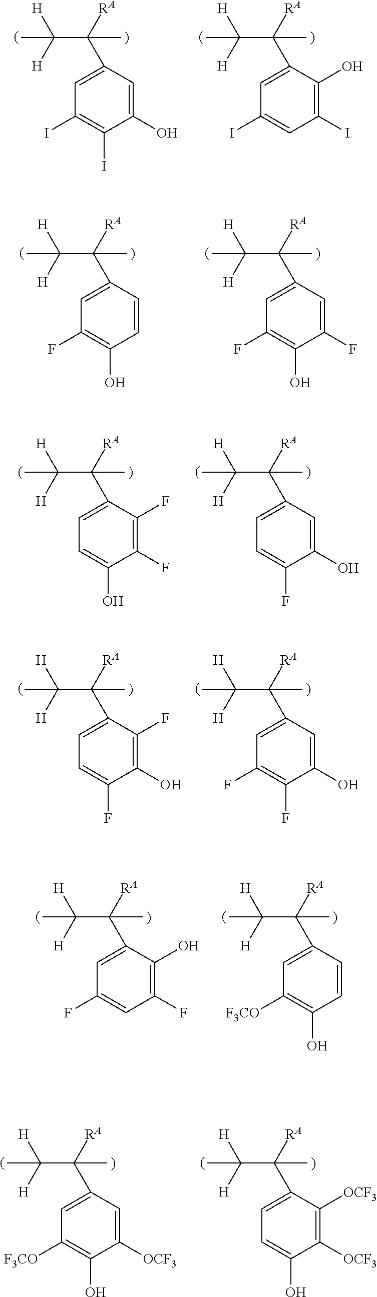

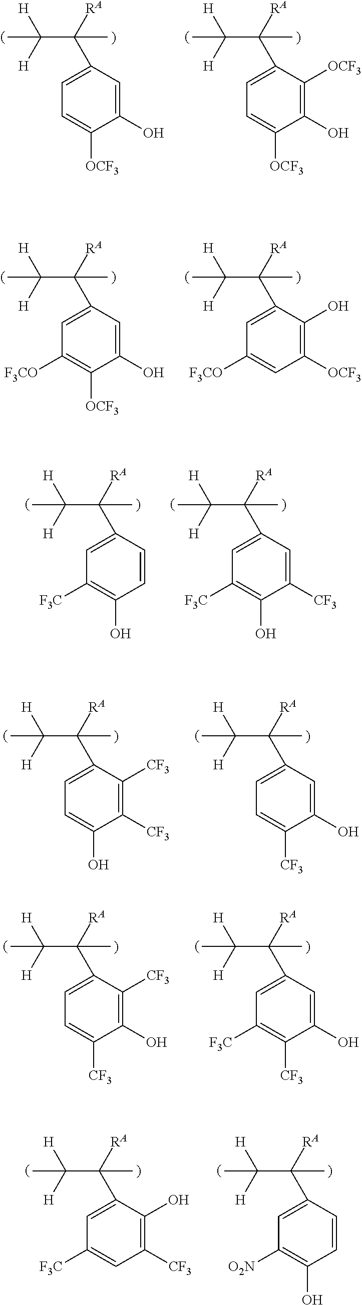

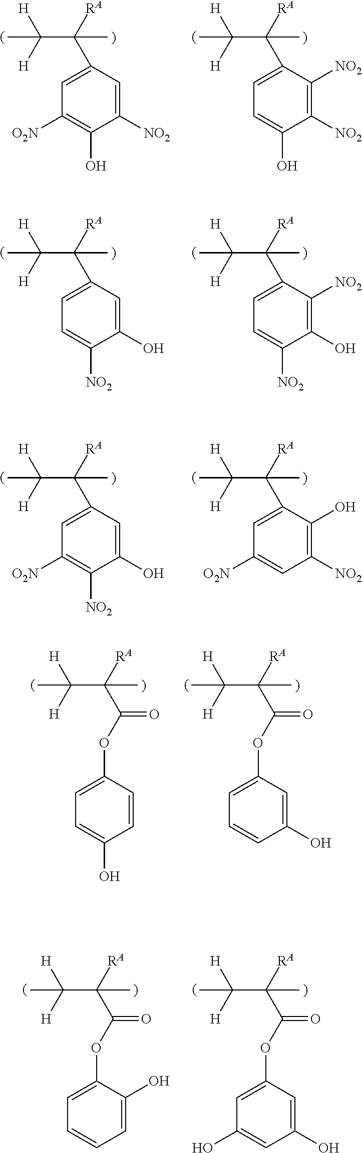

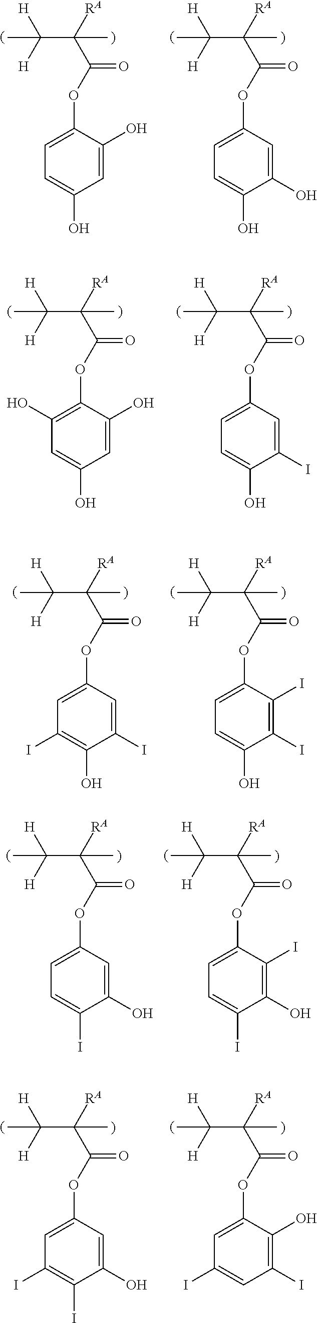

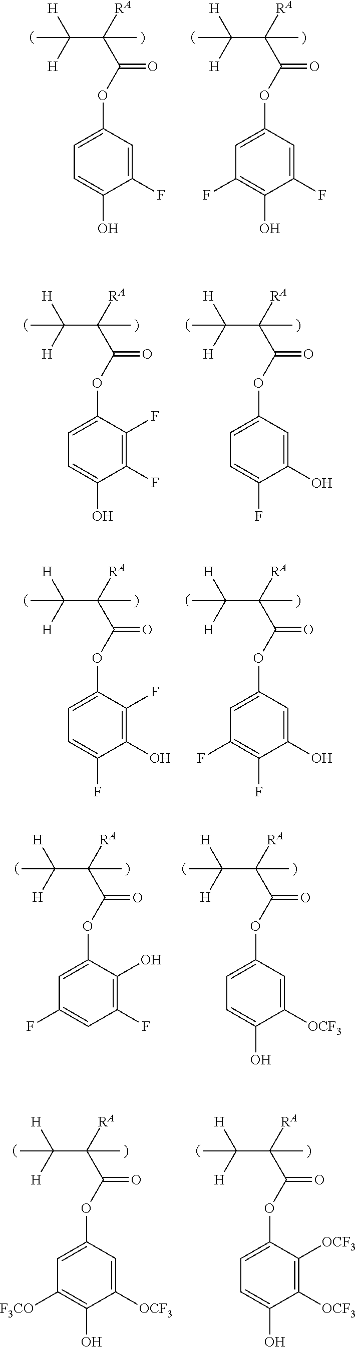

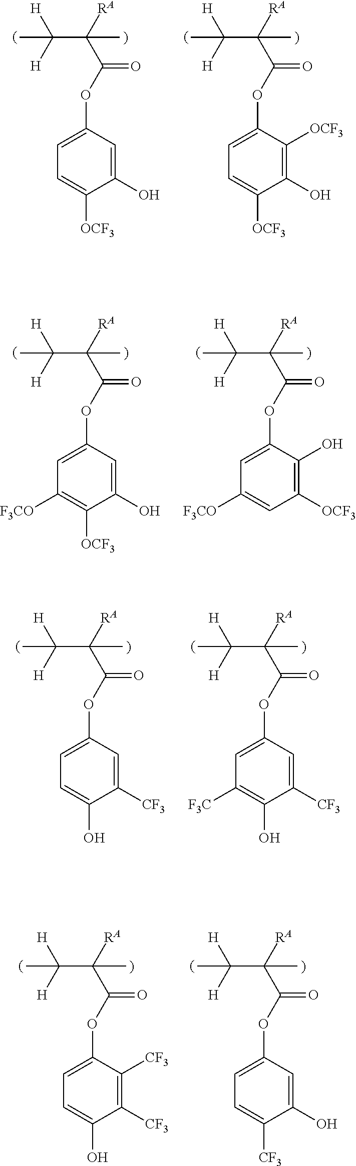

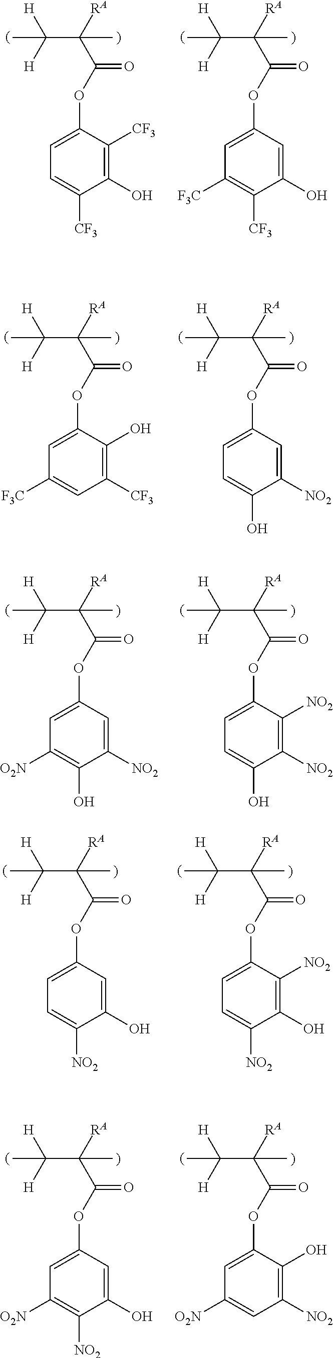

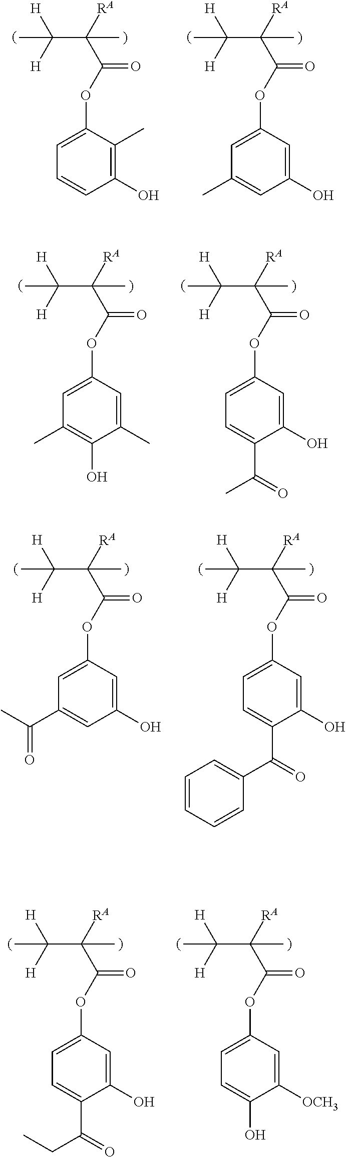

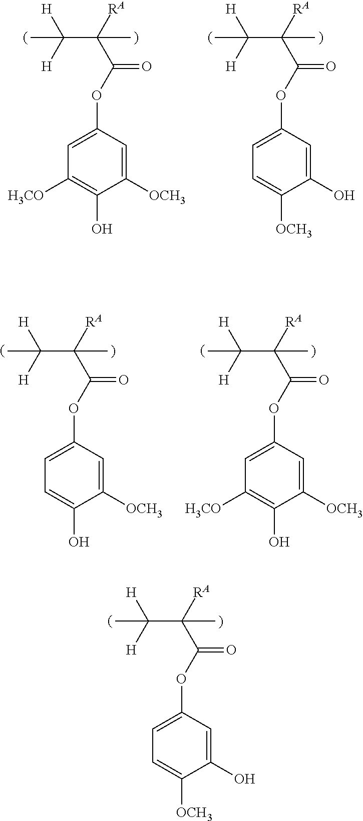

Of the repeat units (c1) and (c2), those units having a lactone ring as the polar group are preferred in the ArF lithography and those units having a phenolic hydroxy group are preferred in the KrF, EB and EUV lithography.

In a preferred embodiment, the polymer further comprises repeat units having a photoacid generating group. This enables to positively control the diffusion of the generated acid, leading to improvements in LWR and critical dimension uniformity (CDU). Examples of the repeat units having a photoacid generating group are repeat units having formula (d1), repeat units having formula (d2), repeat units having formula (d3), and repeat units having formula (d4), which are also referred to as repeat units (d1), (d2), (d3) and (d4).

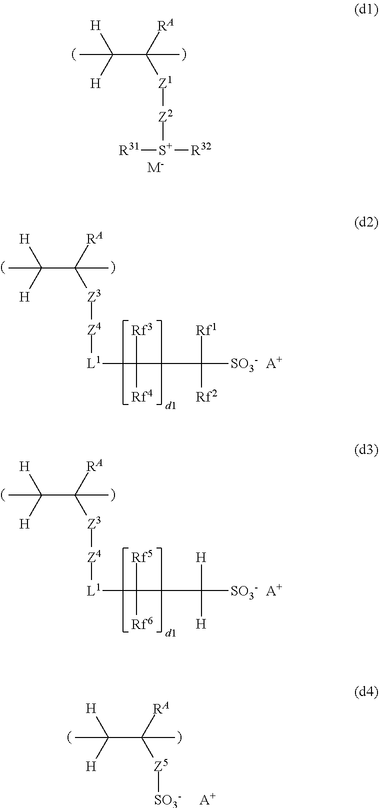

In formulae (d1) to (d4), R^{A} is each independently hydrogen, fluorine, methyl or trifluoromethyl. Z¹ is a single bond or phenylene group. Z² is **—C(═O)—O—Z²¹—, **—C(═O)—NH—Z²¹—, or **—O—Z²¹—. Z²¹ is a C₁-C₆ aliphatic hydrocarbylene group, a phenylene group or a divalent group obtained by combining the foregoing, which may contain at least one selected from a carbonyl moiety, ester bond, ether bond and hydroxy moiety. Z³ is each independently a single bond, phenylene group, naphthylene group or *—C(═O)—O—Z³¹. Z³¹ is a C₁-C₁₀ aliphatic hydrocarbylene group which may contain at least one selected from a hydroxy moiety, ether bond, ester bond and lactone ring, a phenylene group or naphthylene group. Z⁴ is a single bond or ***—Z⁴¹—C(═O)—O—. Z⁴¹ is a C₁-C₂₀ hydrocarbylene group which may contain a heteroatom. Z⁵ is a single bond, methylene, ethylene, phenylene, fluorinated phenylene, trifluoromethyl-substituted phenylene group, *—C(═O)—O—Z⁵¹—, *—C(═O)—N(H)—Z¹¹—, or *—O—Z⁵. Z⁵¹ is a C₁-C₆ aliphatic hydrocarbylene group, phenylene group, fluorinated phenylene group or trifluoromethyl-substituted phenylene group, which may contain at least one selected from a carbonyl moiety, ester bond, ether bond and hydroxy moiety. The asterisk (*) designates a point of attachment to the carbon atom in the backbone. The double asterisks (**) designates a point of attachment to Z¹. The triple asterisks (***) designates a point of attachment to Z³.

The aliphatic hydrocarbylene group represented by Z²¹, Z³¹ and Z⁵¹ may be straight, branched or cyclic. Examples thereof include alkanediyl groups such as methanediyl, ethane-1,1-diyl, ethane-1,2-diyl, propane-1,1-diyl, propane-1,2-diyl, propane-1,3-diyl, propane-2,2-diyl, butane-1,1-diyl, butane-1,2-diyl, butane-1,3-diyl, butane-2,3-diyl, butane-1,4-diyl, 1,1-dimethylethane-1,2-diyl, pentane-1,5-diyl, 2-methylbutane-1,2-diyl, and hexane-1,6-diyl; cycloalkanediyl groups such as cyclopropanediyl, cyclobutanediyl, cyclopentanediyl, and cyclohexanediyl; and combinations thereof.

The hydrocarbylene group represented by Z⁴¹ may be saturated or unsaturated and straight, branched or cyclic. Examples thereof are shown below, but not limited thereto.

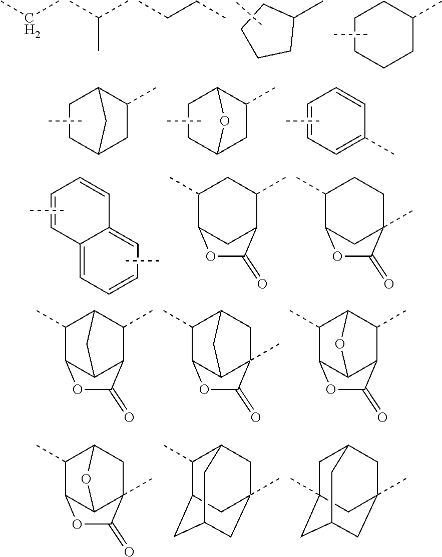

Herein the broken line is a bond.

In formula (d1), R³¹ and R³² are each independently a C₁-C₂₀ hydrocarbyl group which may contain a heteroatom. R³¹ and R³² may bond together to form a ring with the sulfur atom to which they are attached. The hydrocarbyl group may be saturated or unsaturated and straight, branched or cyclic. Examples thereof include C₁-C₂₀ alkyl groups such as methyl, ethyl, n-propyl, isopropyl, n-butyl, isobutyl, sec-butyl, and tert-butyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, allyl, propenyl, butenyl, and hexenyl; C₃-C₂₀ cyclic unsaturated hydrocarbyl groups such as cyclohexenyl; C₆-C₂₀ aryl groups such as phenyl, naphthyl and thienyl; C₇-C₂₀ aralkyl groups such as benzyl, 1-phenylethyl and 2-phenylethyl; and combinations thereof. Inter alia, aryl groups are preferred. In the foregoing hydrocarbyl groups, some or all of the hydrogen atoms may be substituted by a moiety containing a heteroatom such as oxygen, sulfur, nitrogen or halogen, or some constituent —CH₂— may be substituted by a moiety containing a heteroatom such as oxygen, sulfur or nitrogen, so that the group may contain a hydroxy, fluorine, chlorine, bromine, iodine, cyano, carbonyl, ether bond, ester bond, sulfonic ester bond, carbonate bond, lactone ring, sultone ring, carboxylic anhydride (—C(═O)—O—C(═O)—) or haloalkyl moiety.

Illustrative examples of the cation in the repeat unit (d1) are shown below, but not limited thereto. Herein R^{A} is as defined above.

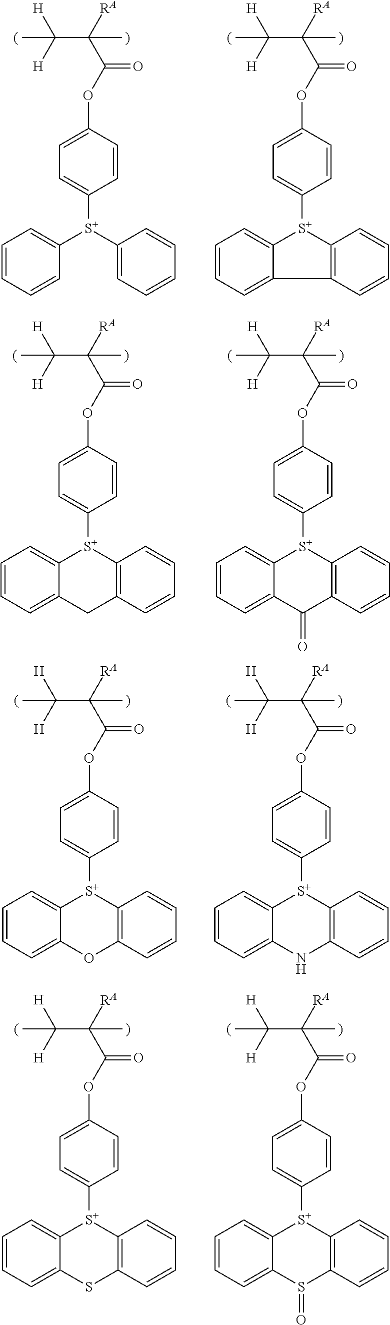

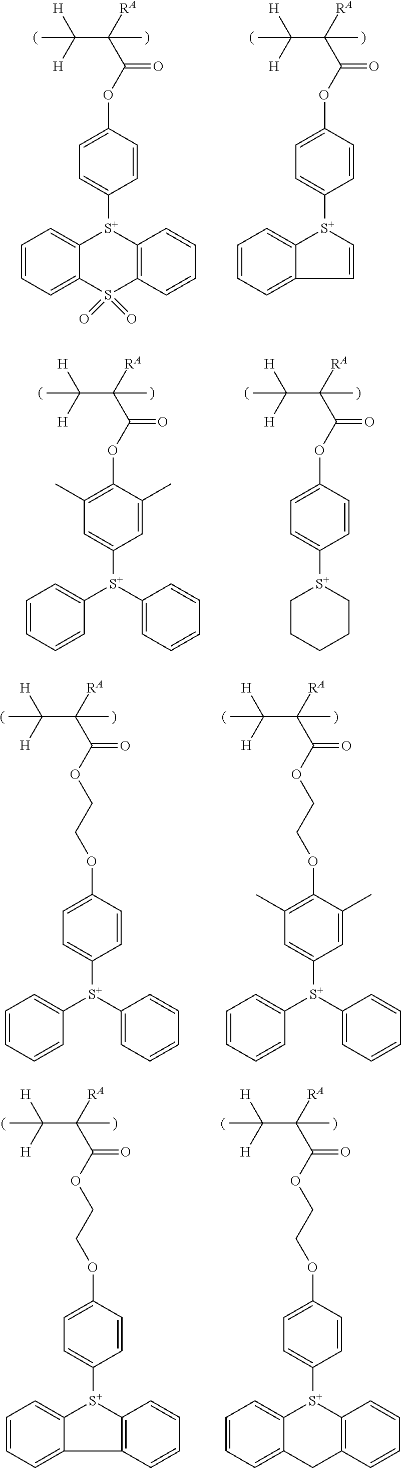

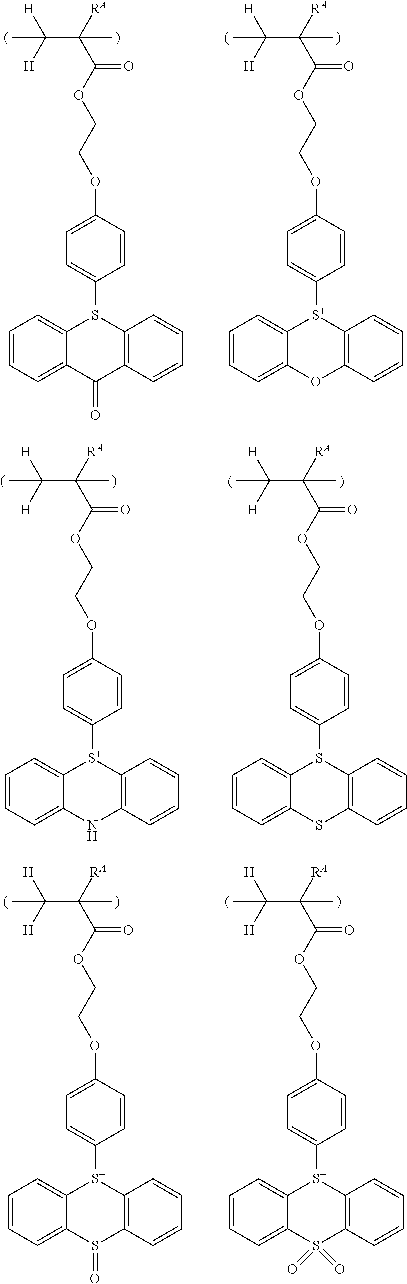

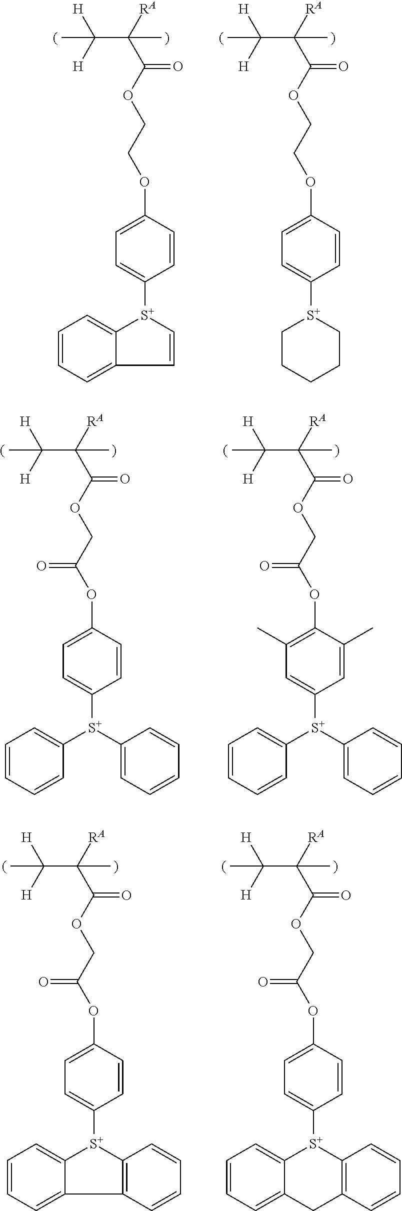

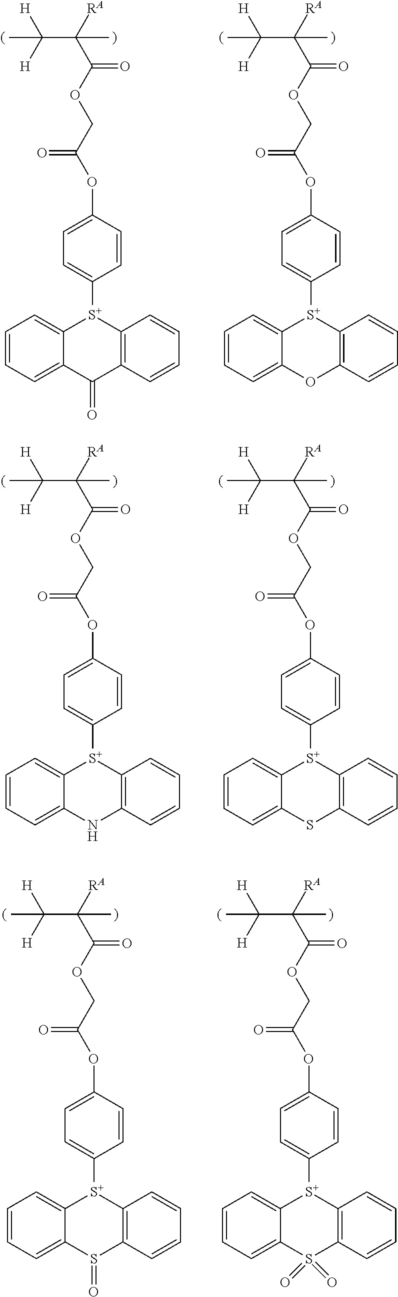

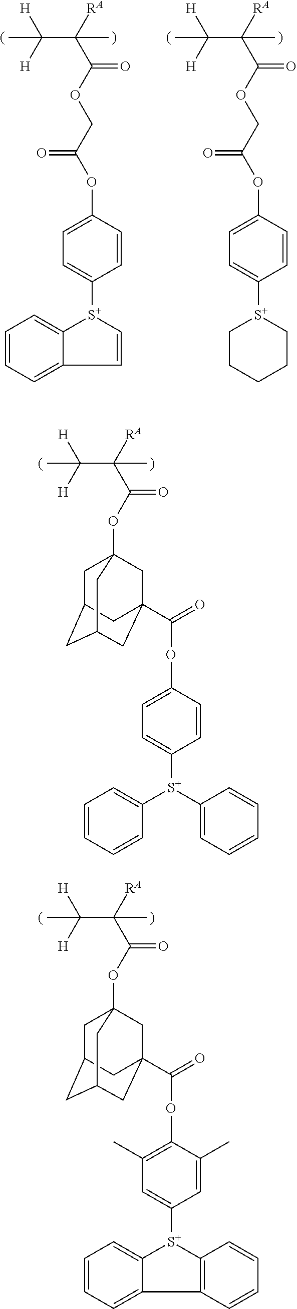

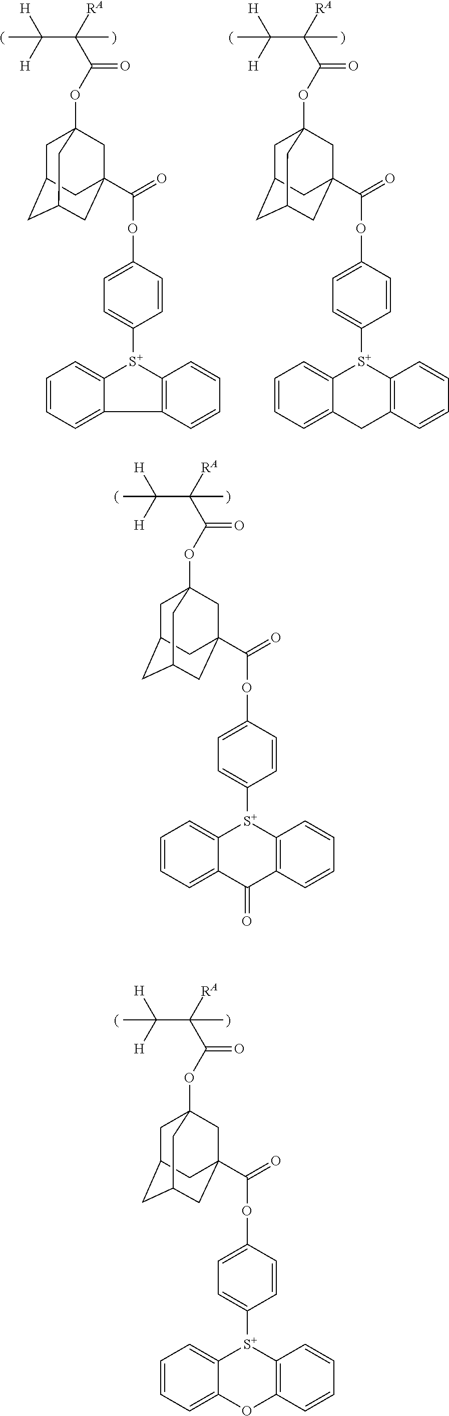

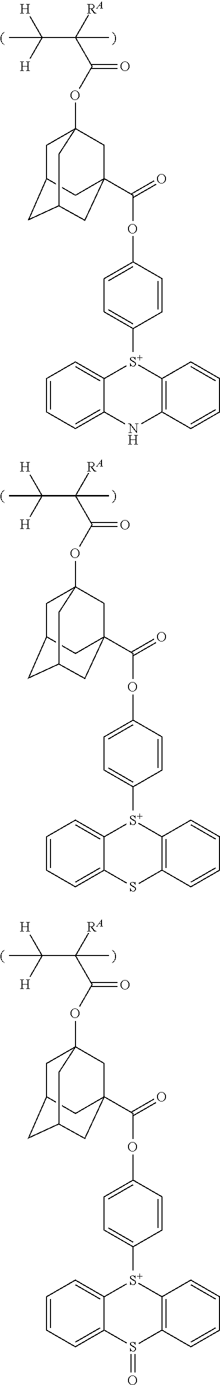

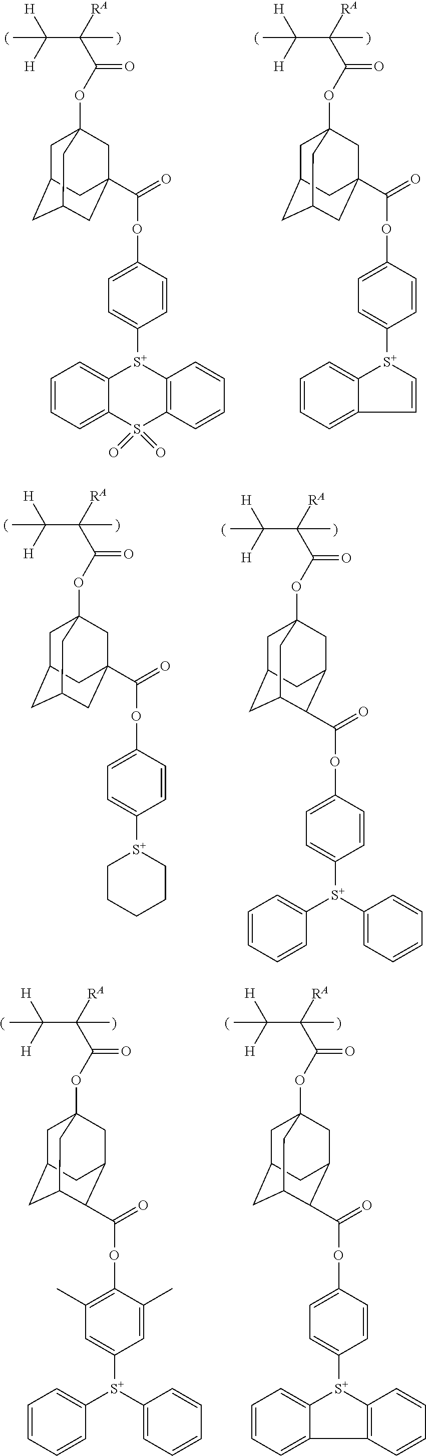

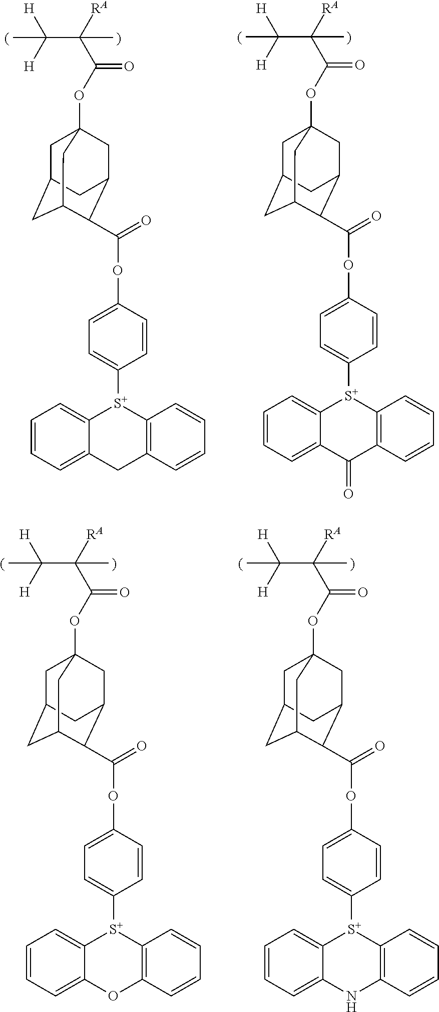

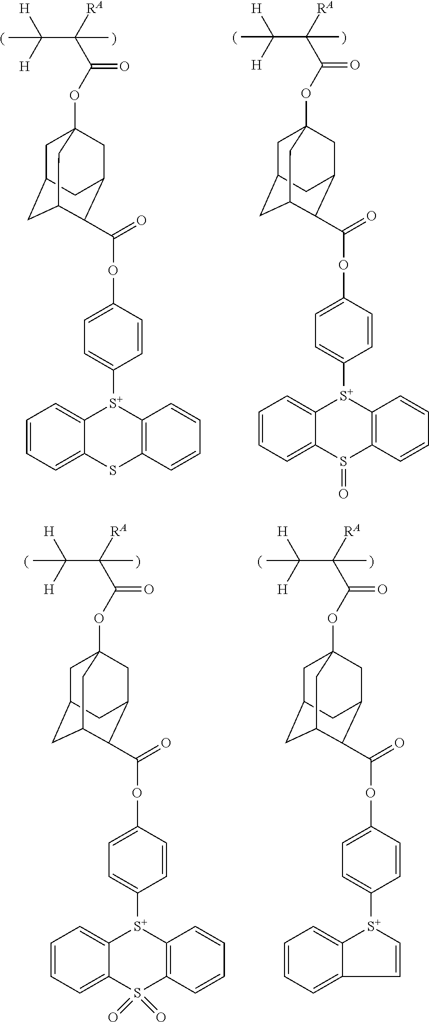

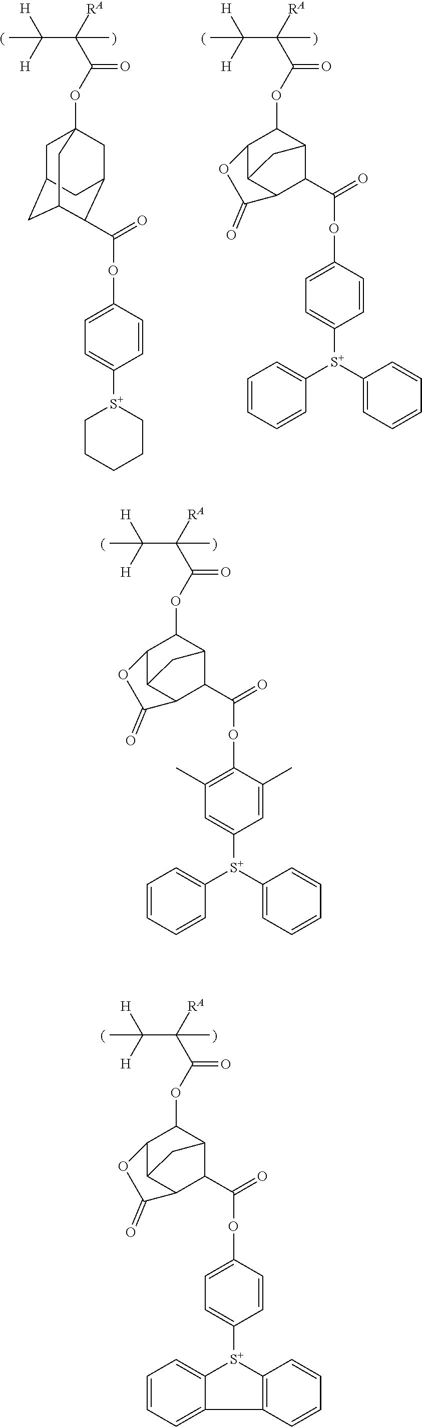

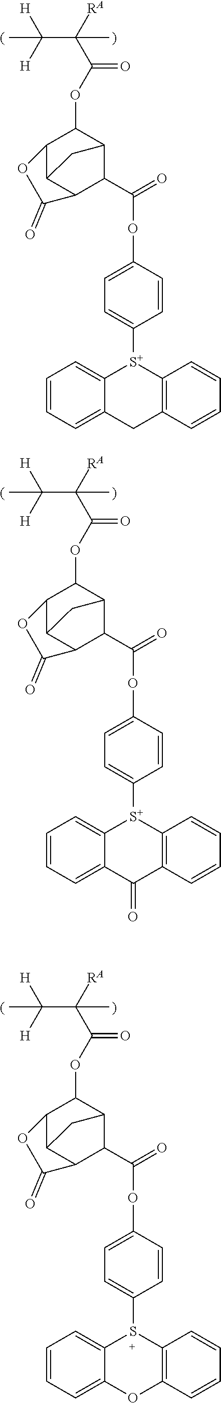

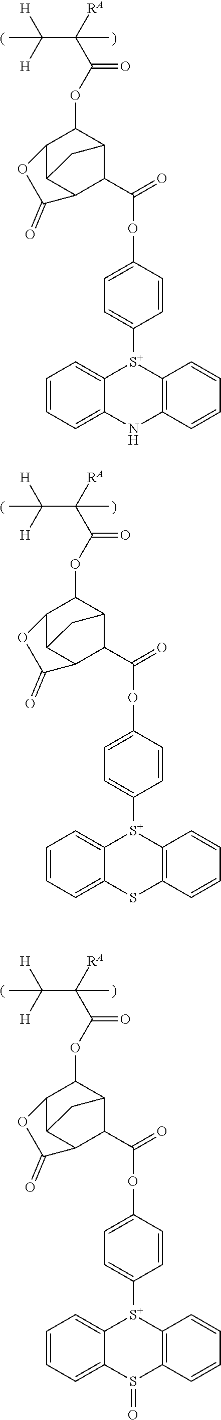

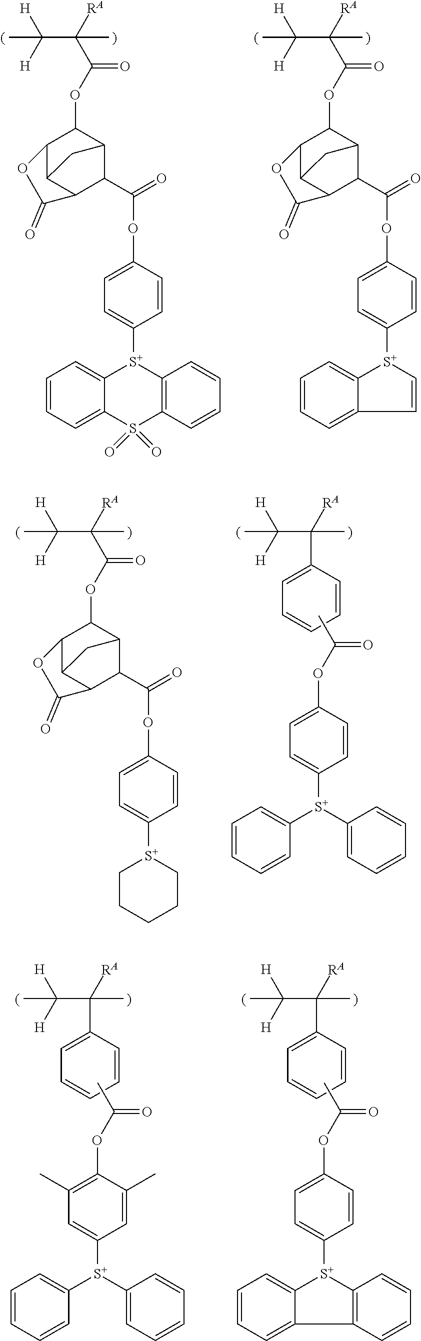

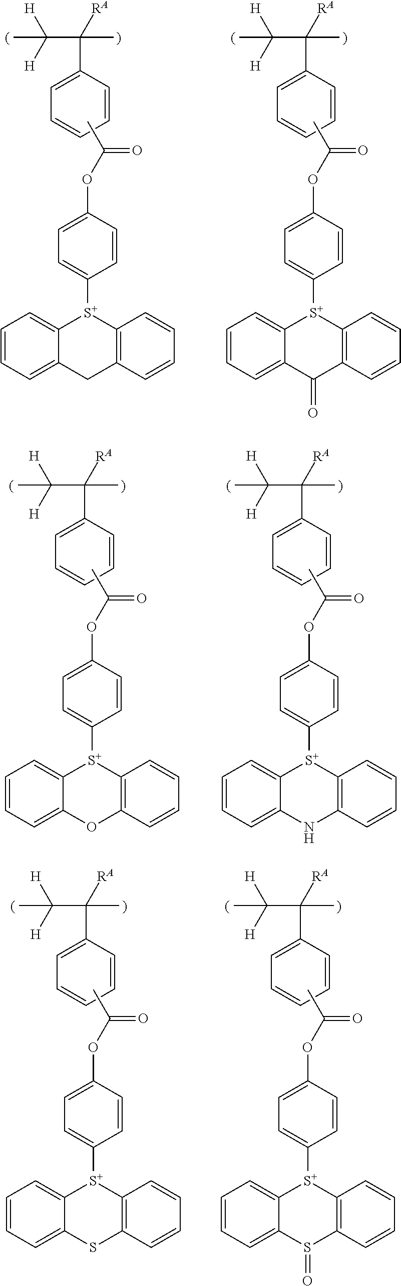

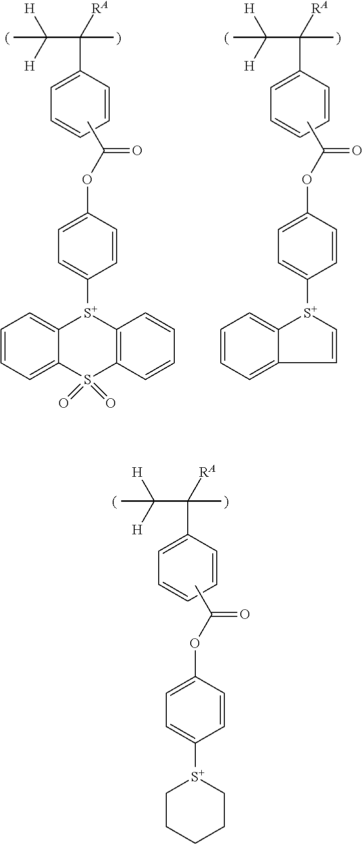

In formula (d1), M⁻ is a non-nucleophilic counter ion. Examples of the non-nucleophilic counter ion include halide ions such as chloride and bromide ions; fluoroalkylsulfonate ions such as triflate, 1,1,1-trifluoroethanesulfonate, and nonafluorobutanesulfonate; arylsulfonate ions such as tosylate, benzenesulfonate, 4-fluorobenzenesulfonate, and 1,2,3,4,5-pentafluorobenzenesulfonate; alkylsulfonate ions such as mesylate and butanesulfonate; imide ions such as bis(trifluoromethylsulfonyl)imide, bis(perfluoroethylsulfonyl)imide and bis(perfluorobutylsulfonyl)imide; and methide ions such as tris(trifluoromethylsulfonyl)methide and tris(perfluoroethylsulfonyl)methide.

Other examples of the non-nucleophilic counter ion include a sulfonate anion which is fluorinated at α-position as represented by formula (d1-1) and a sulfonate anion which is substituted with fluorine at α-position and trifluoromethyl at β-position as represented by formula (d1-2).

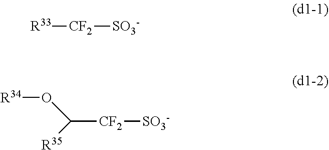

In formula (d1-1), R³³ is hydrogen, a C₁-C₃₀ hydrocarbyl group, C₂-C₃₀ hydrocarbylcarbonyloxy group, or C₂-C₃₀ hydrocarbyloxycarbonyl group. The hydrocarbyl group may contain halogen, an ether bond, ester bond, carbonyl moiety or lactone ring. The hydrocarbyl group and hydrocarbyl moiety in the hydrocarbylcarbonyloxy group and hydrocarbyloxycarbonyl group may be saturated or unsaturated and straight, branched or cyclic. Examples thereof are as will be exemplified later for the hydrocarbyl group R^{fa1} in formula (2A′).

In formula (d1-2), R³⁴ is hydrogen, a C₁-C₃₀ hydrocarbyl group, or C₂-C₃₀ hydrocarbylcarbonyl group. The hydrocarbyl group and hydrocarbylcarbonyl group may contain halogen, an ether bond, ester bond, carbonyl moiety or lactone ring. R³⁵ is hydrogen, fluorine or a C₁-C₆ fluorinated alkyl group. The hydrocarbyl group and hydrocarbyl moiety in the hydrocarbylcarbonyl group may be saturated or unsaturated and straight, branched or cyclic. Examples thereof are as will be exemplified later for the hydrocarbyl group R^{fa1} in formula (2A′). R³⁵ is preferably trifluoromethyl.

Examples of the sulfonate anion having formula (d1-1) or (d1-2) are shown below, but not limited thereto. Herein R³⁵ is as defined above, and Ac is acetyl.

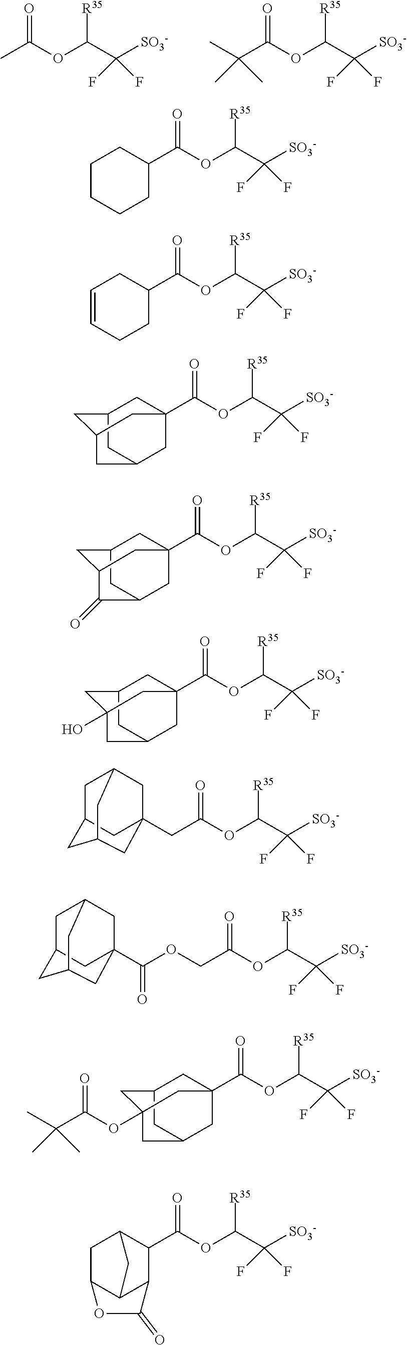

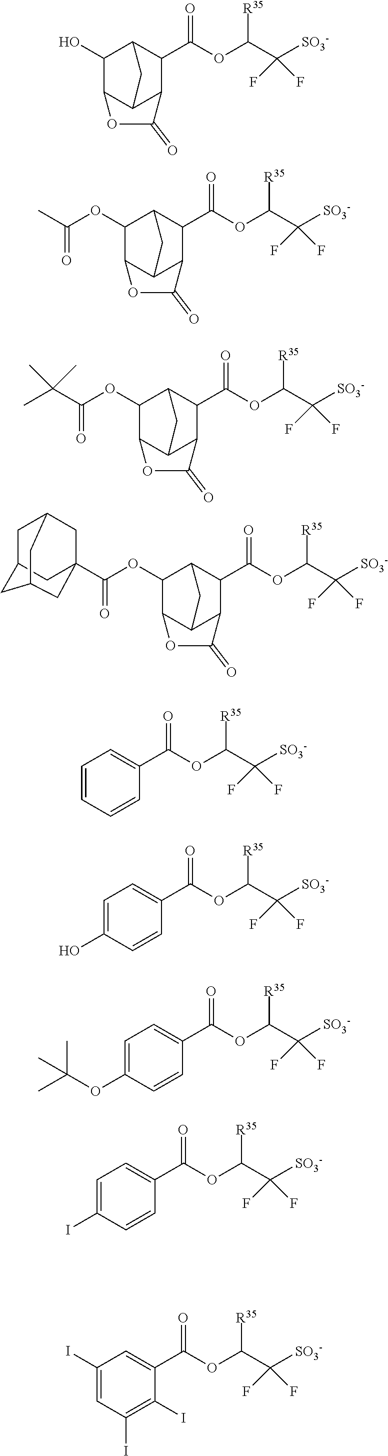

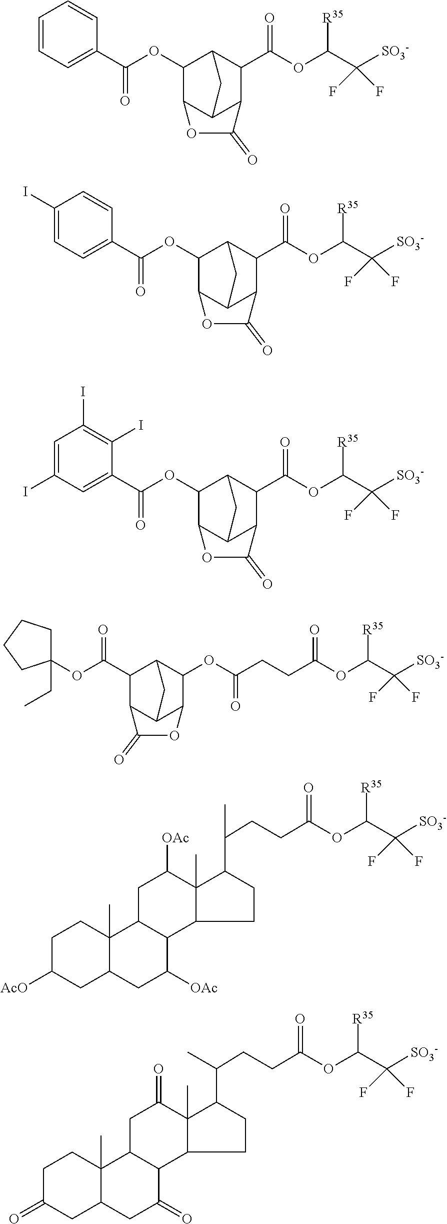

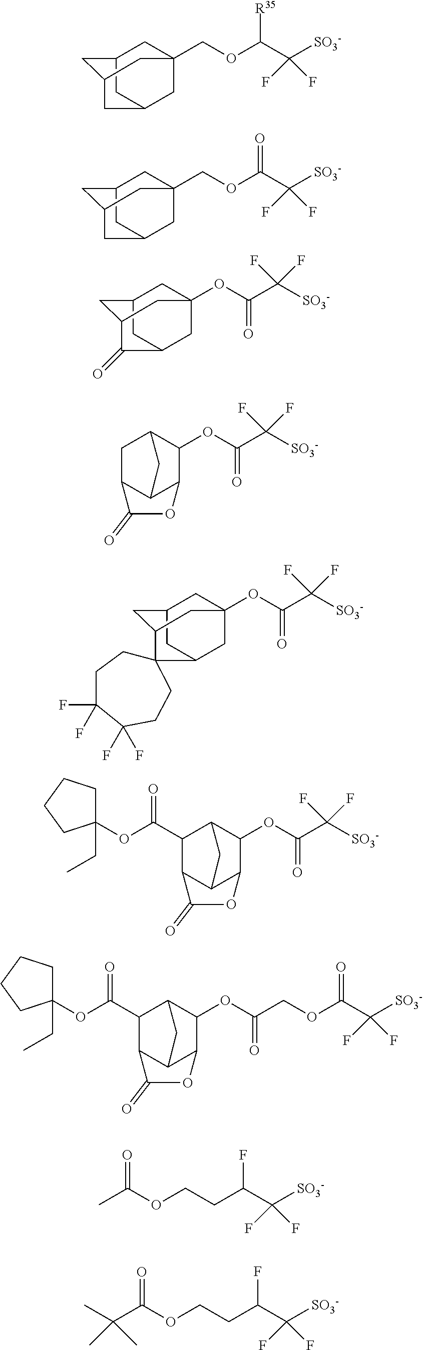

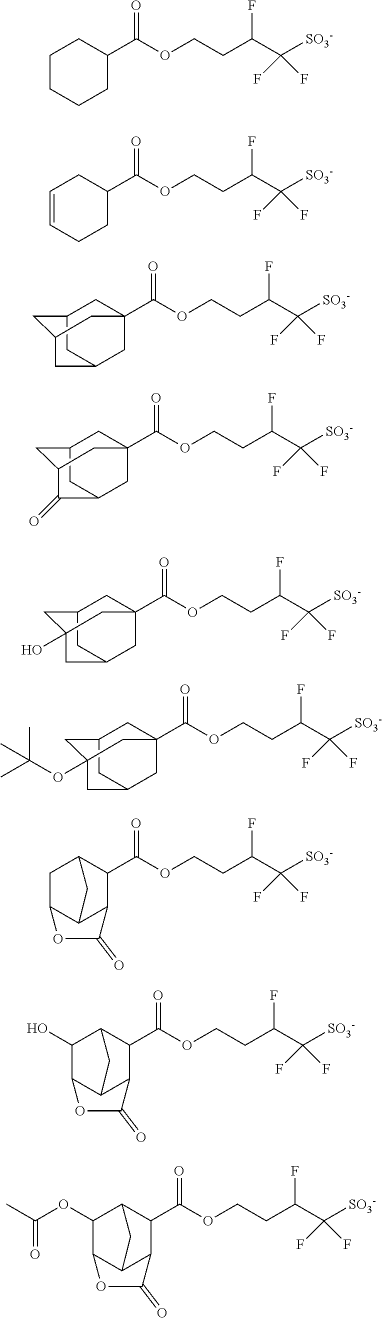

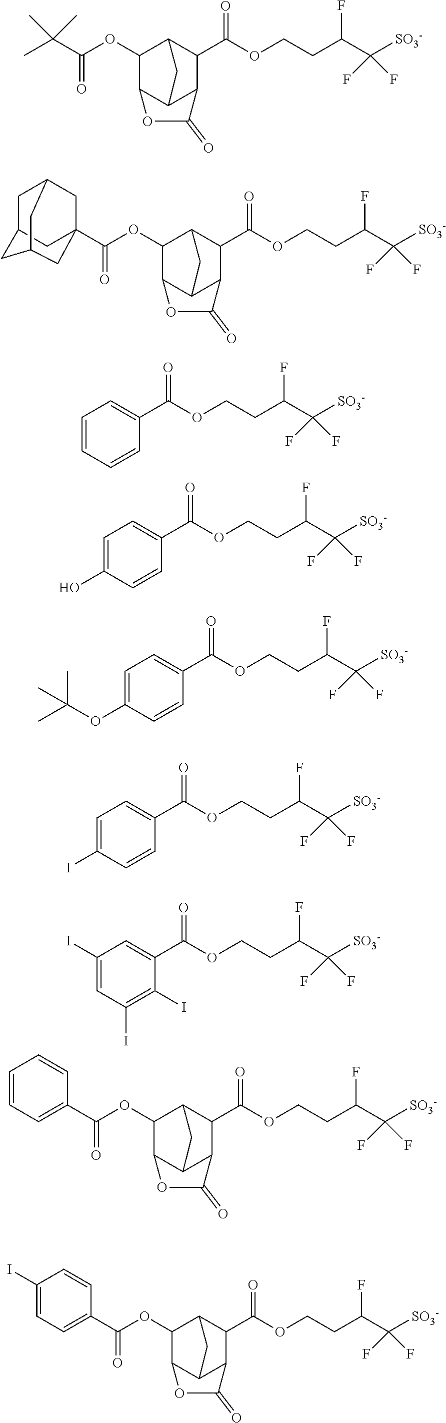

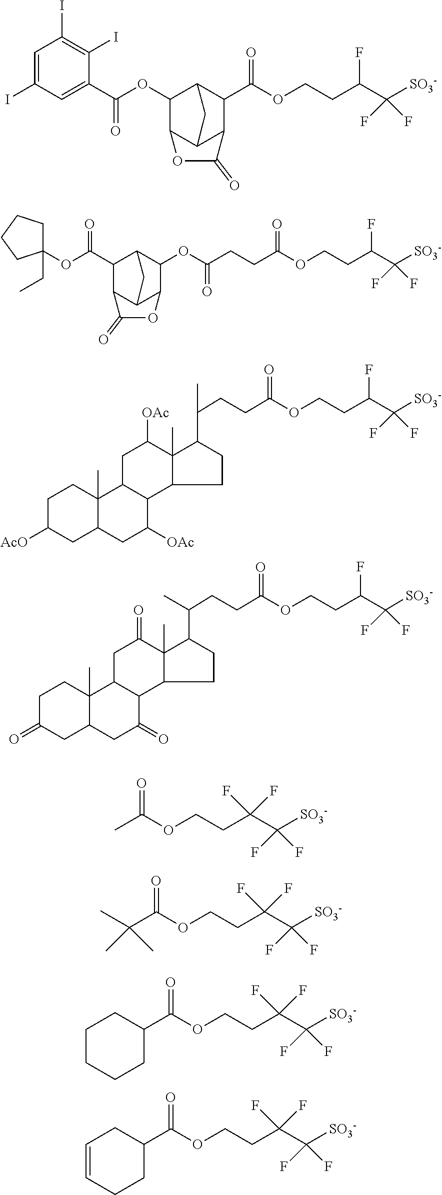

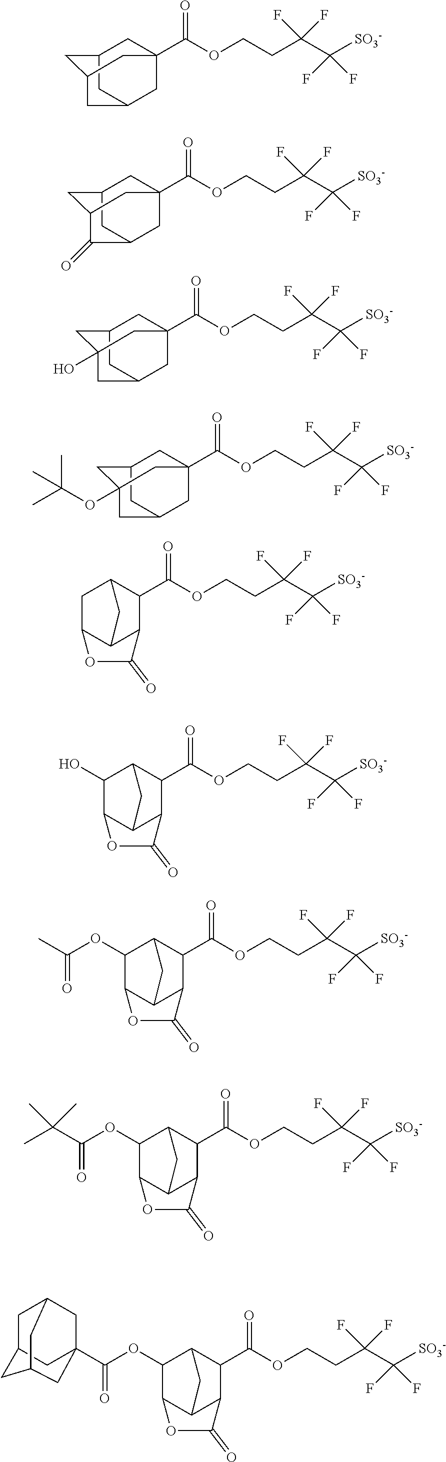

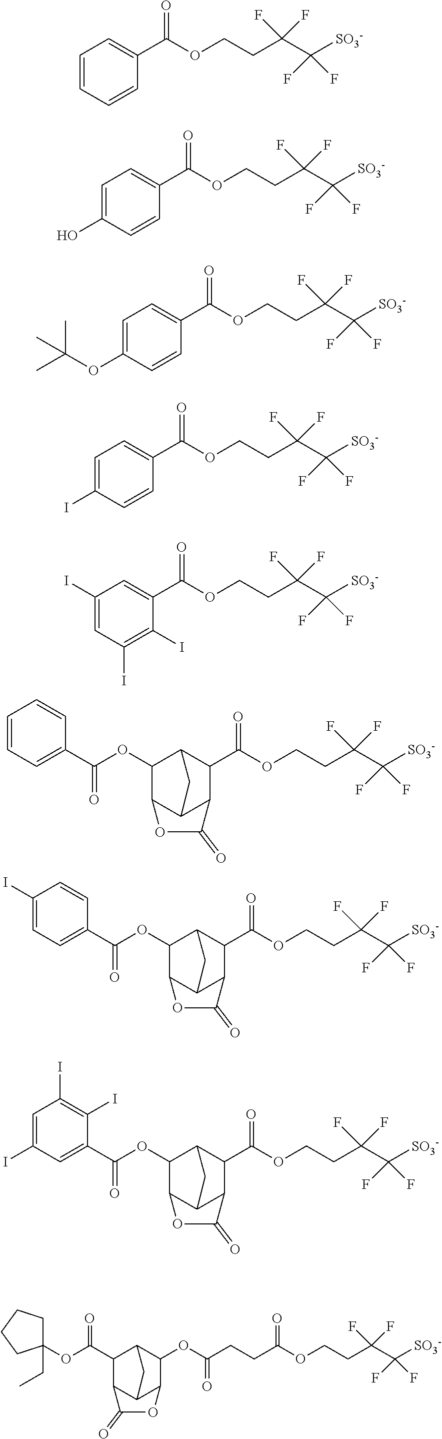

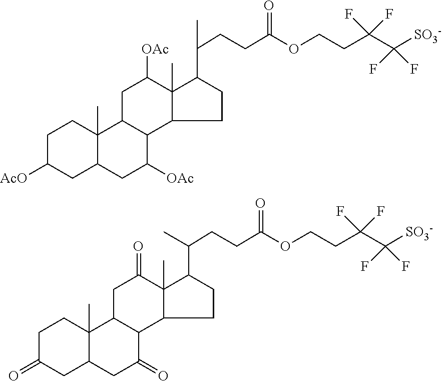

In formulae (d2) and (B), L¹ is a single bond, ether bond, ester bond, carbonyl group, sulfonic ester bond, carbonate bond or carbamate bond. From the aspect of synthesis, an ether bond, ester bond, and carbonyl group are preferred, with an ester bond and carbonyl group being more preferred.

In formula (d2), Rf¹ and Rf² are each independently fluorine or a C₁-C₆ fluorinated alkyl group. It is preferred for enhancing the acid strength of the generated acid that both Rf¹ and Rf² be fluorine. Rf³ and Rf⁴ are each independently hydrogen, fluorine or a C₁-C₆ fluorinated alkyl group. It is preferred for enhancing solvent solubility that at least one of Rf³ and Rf⁴ be trifluoromethyl.

In formula (d3), Rf⁵ and Rf⁶ are each independently hydrogen, fluorine or a C₁-C₆ fluorinated alkyl group. It is excluded that all Rf⁵ and Rf⁶ are hydrogen at the same time. It is preferred for enhancing solvent solubility that at least one of Rf⁵ and Rf⁶ be trifluoromethyl.

In formulae (d2) and (d3), d is an integer of 0 to 3, preferably 1.

Examples of the anion in the repeat unit (d2) are shown below, but not limited thereto. Herein R^{A} is as defined above.

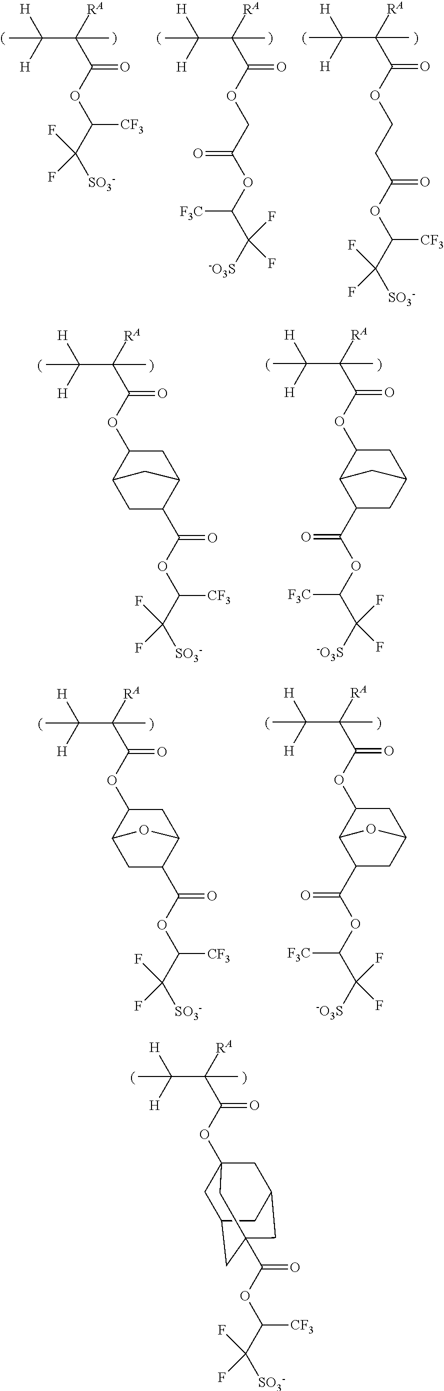

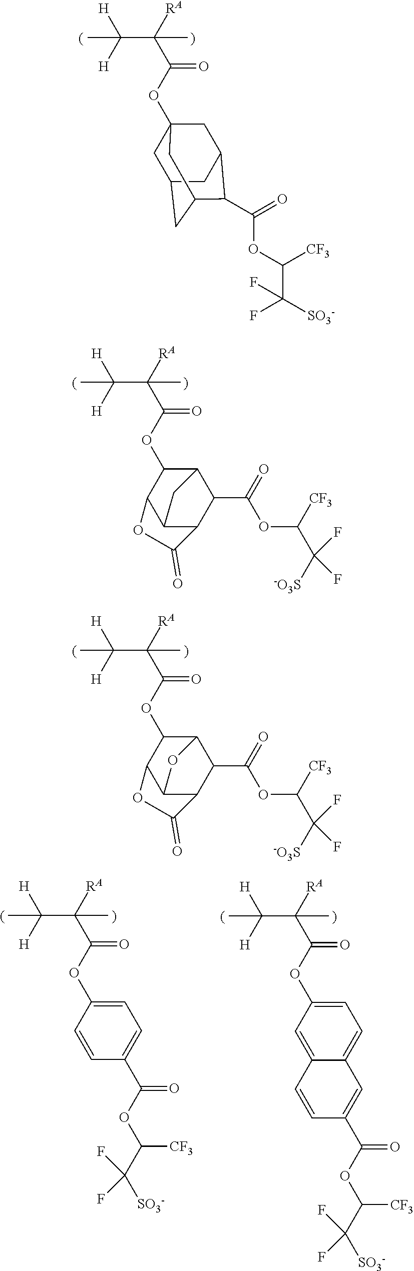

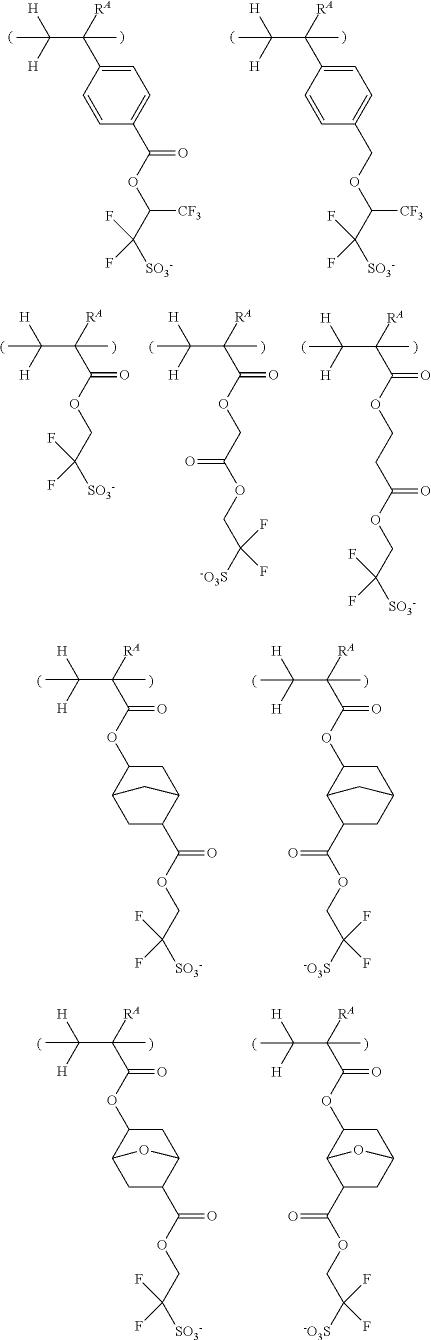

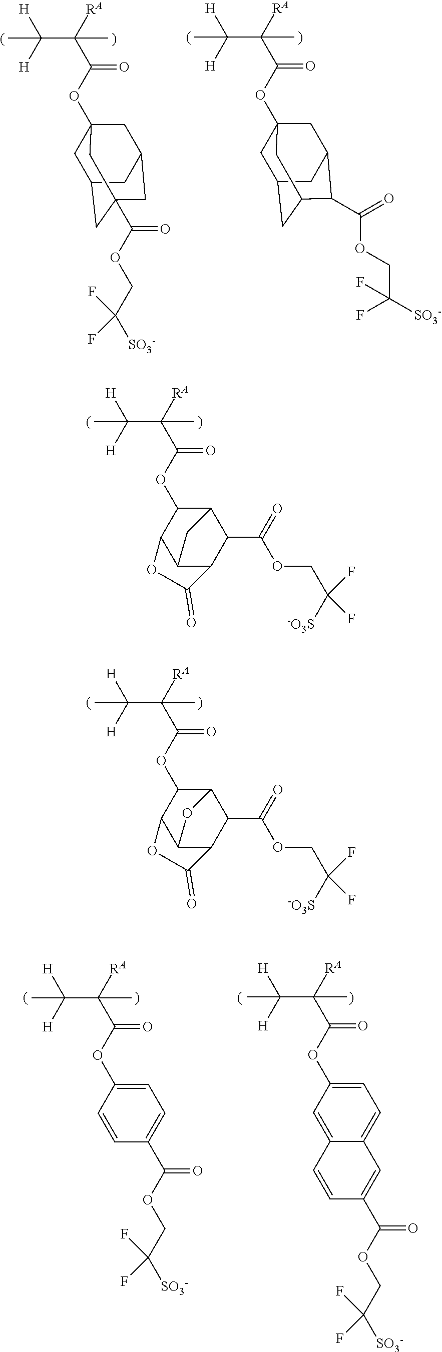

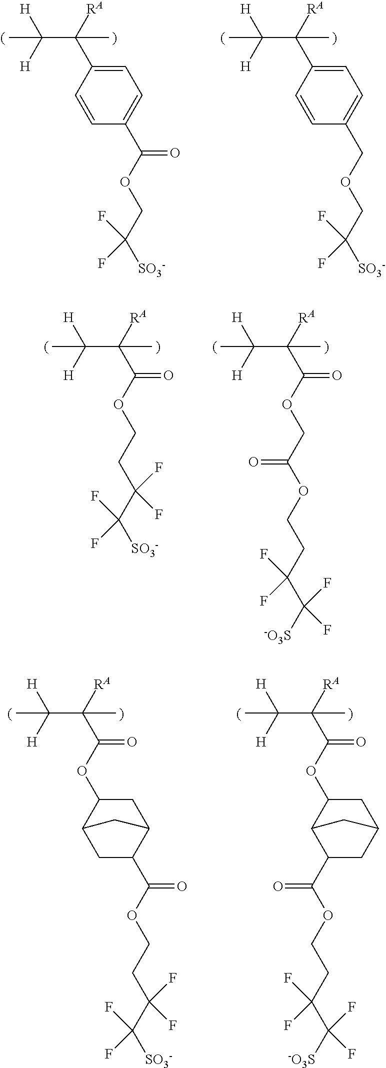

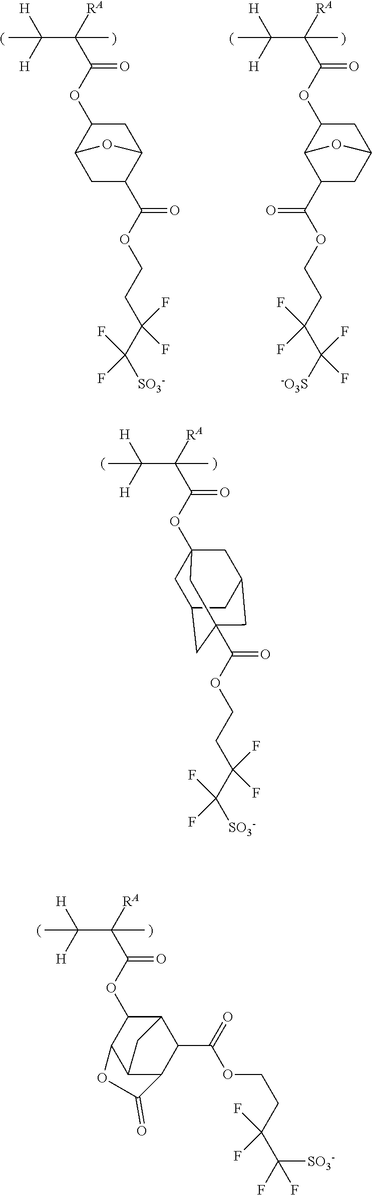

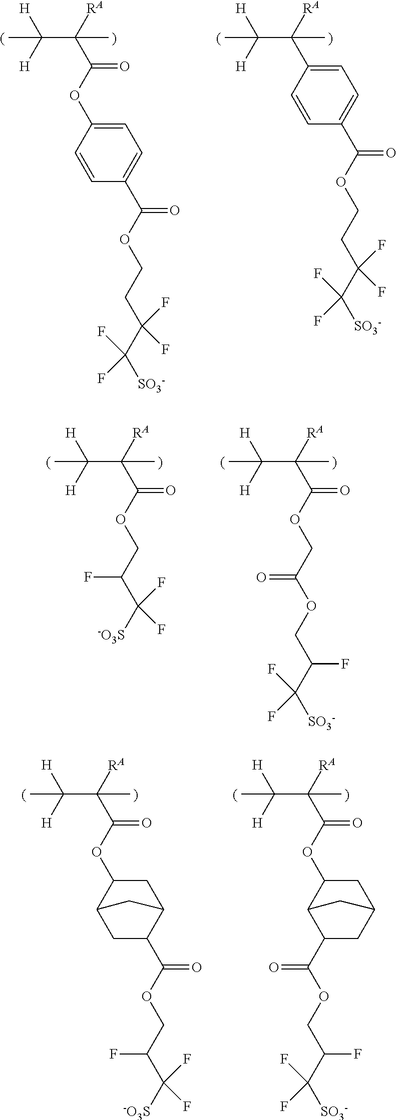

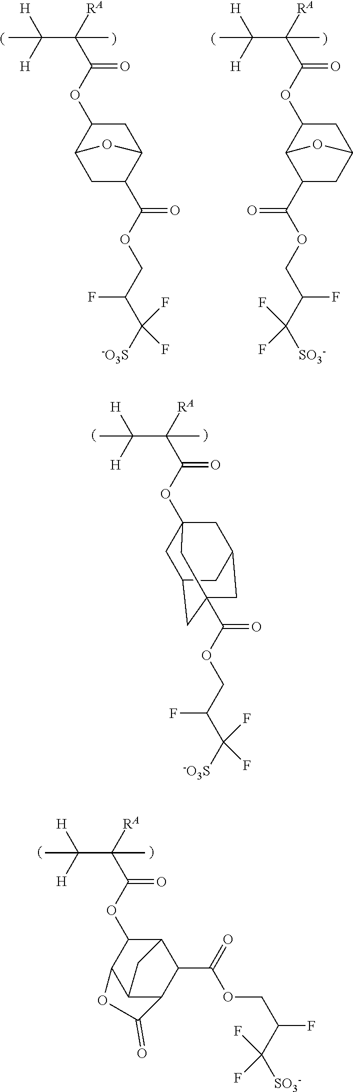

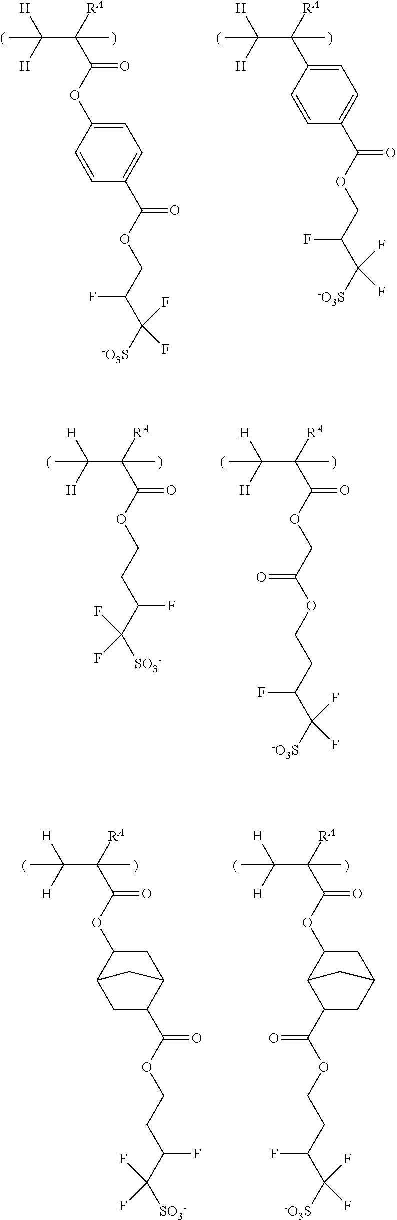

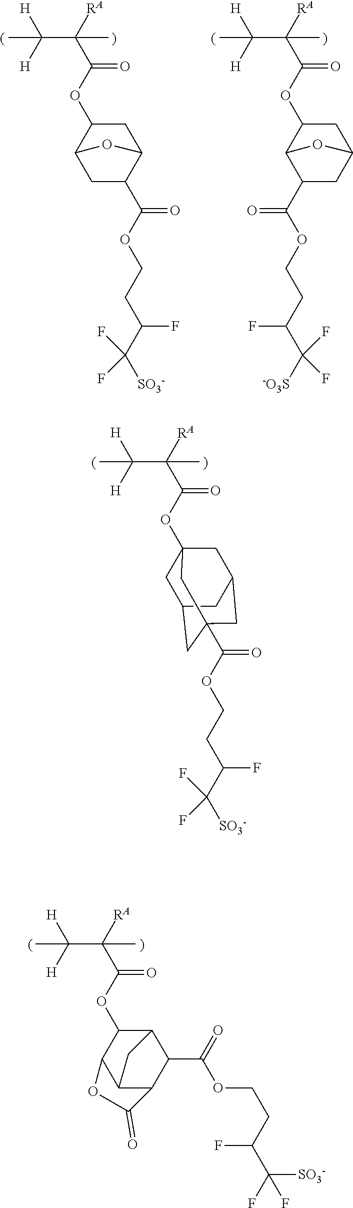

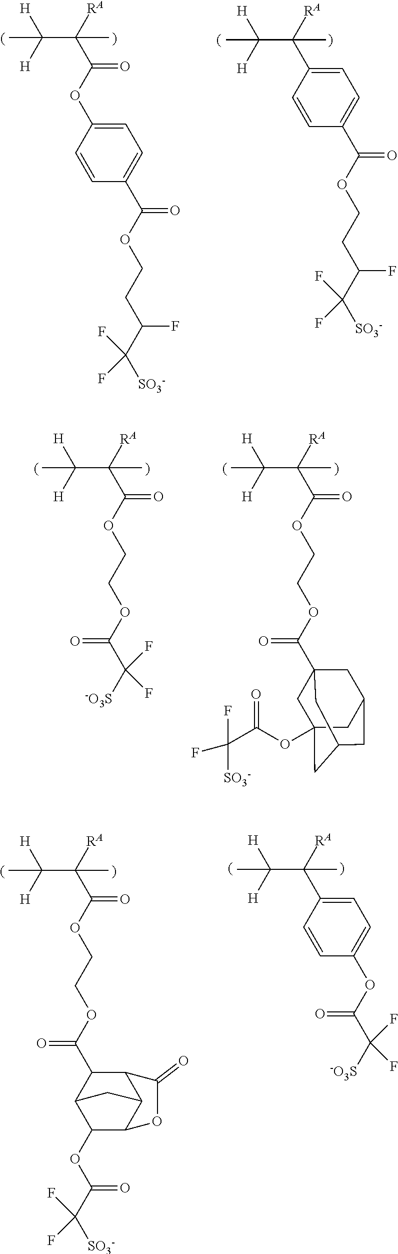

Examples of the anion in the repeat unit (d3) are shown below, but not limited thereto. Herein R^{A} is as defined above.

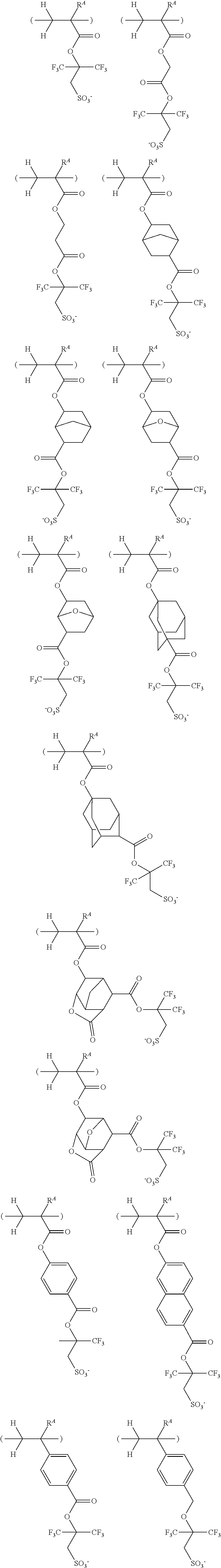

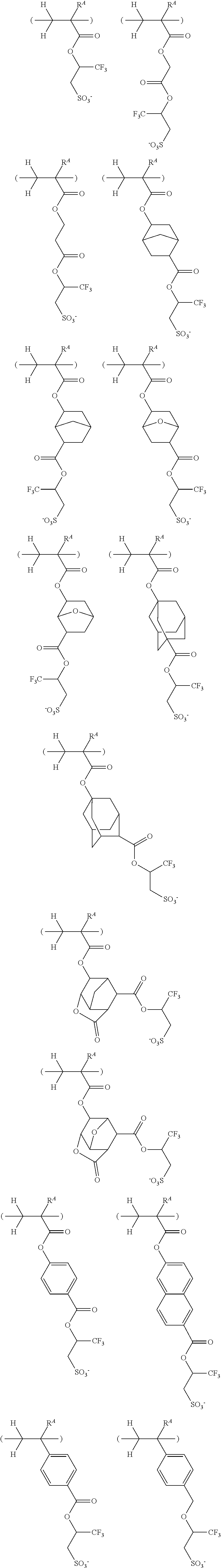

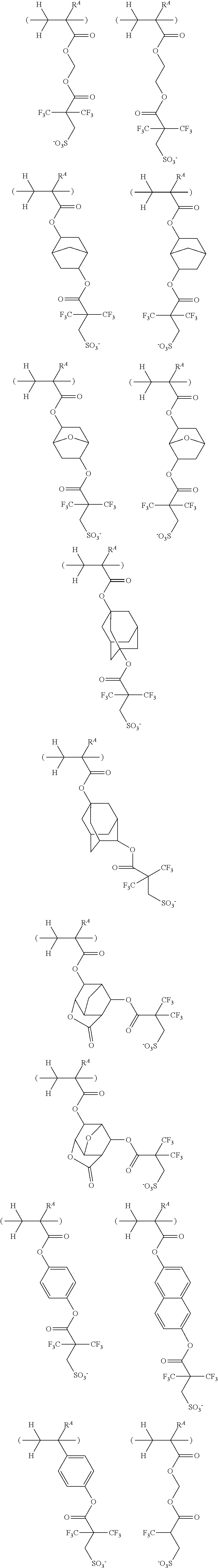

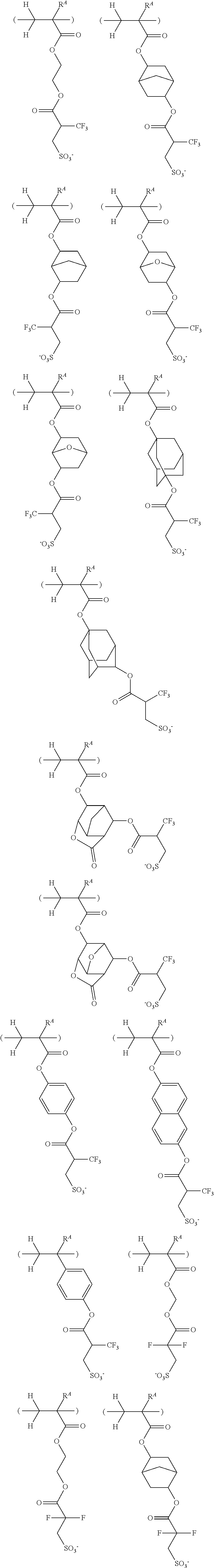

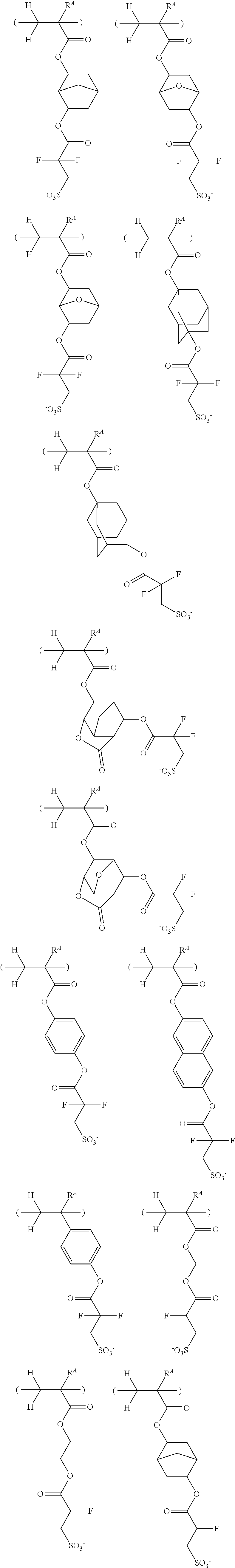

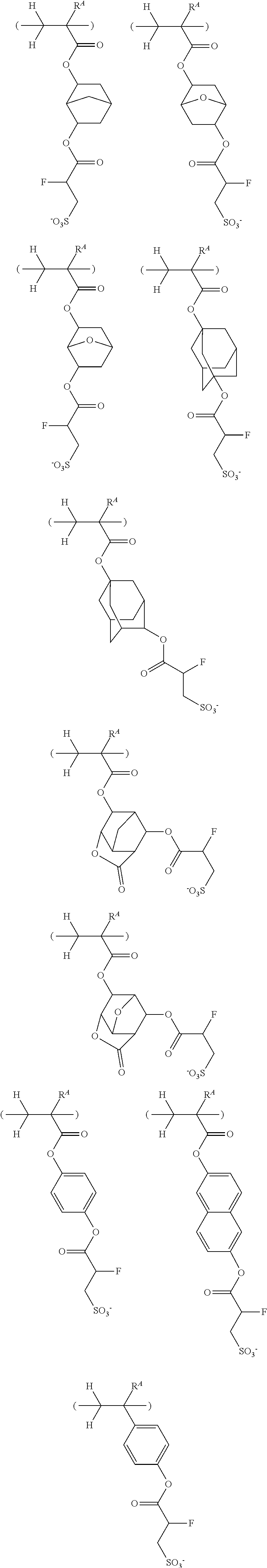

Examples of the anion in the repeat unit (d4) are shown below, but not limited thereto. Herein R^{A} is as defined above.

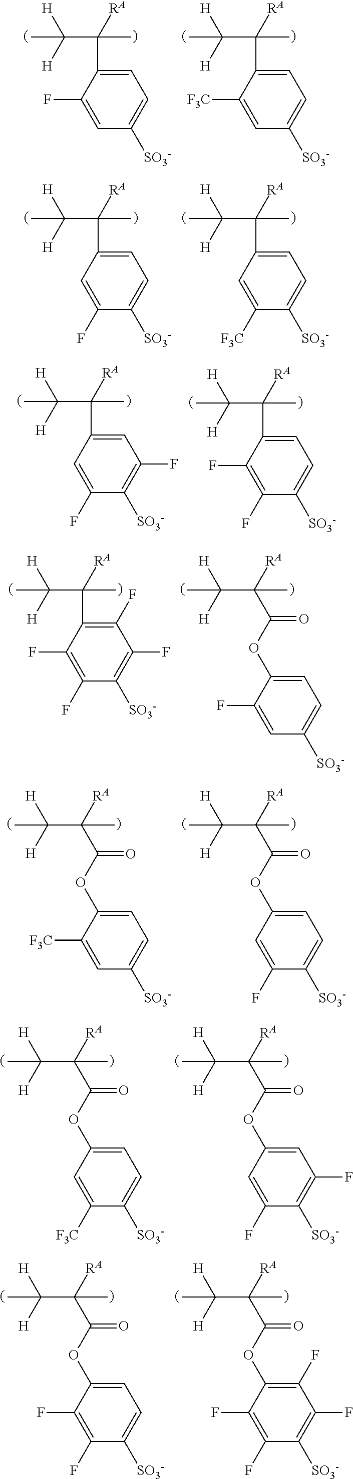

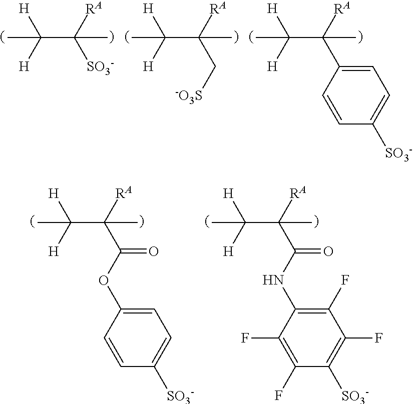

In formulae (d2) to (d4), A⁺ is an onium cation. Suitable onium cations include sulfonium, iodonium and ammonium cations, with the sulfonium and iodonium cations being preferred. Exemplary are cations having formulae (6-1) to (6-3) which are described later.

The repeat units (d1) to (d4) function as a photoacid generator. Where a polymer comprising any of the repeat units (d1) to (d4), that is, polymer-bound photoacid generator is used, the resist composition of the invention may or may not contain (D) a photoacid generator.

The polymer may further comprise repeat units of a structure having a hydroxy group protected with an acid labile group, the repeat units also being referred to as repeat units (e). The repeat unit (e) is not particularly limited as long as the unit includes one or more structures having a hydroxy group protected with a protective group such that the protective group is decomposed to generate the hydroxy group under the action of acid. Repeat units having formula (e1) are preferred.

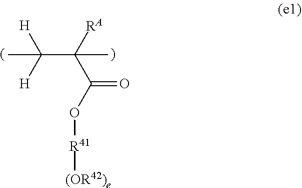

In formula (e1), R^{A} is as defined above. e is an integer of 1 to 4. R⁴¹ is a C₁-C₃₀ (e+1)-valent hydrocarbon group which may contain a heteroatom. R⁴² is an acid labile group.

In formula (e1), the acid labile group R⁴² is deprotected under the action of acid so that a hydroxy group is generated. Although the structure of R⁴² is not particularly limited, an acetal structure, ketal structure, alkoxycarbonyl group and alkoxymethyl group having the following formula (e2) are preferred, with the alkoxymethyl group having formula (e2) being more preferred.

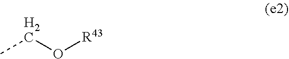

Herein * is a bond, and R⁴³ is a C₁-C₁₅ hydrocarbyl group.

Illustrative examples of the acid labile group R⁴², the alkoxymethyl group having formula (e2), and the repeat units (e) are as exemplified for the repeat units (d) in JP-A 2020-111564 (US 20200223796).

In addition to the foregoing units, the polymer may further comprise repeat units (f) derived from indene, benzofuran, benzothiophene, acenaphthylene, chromone, coumarin, and norbornadiene, or derivatives thereof. Examples of the monomer from which the repeat units (f) are derived are shown below, but not limited thereto.

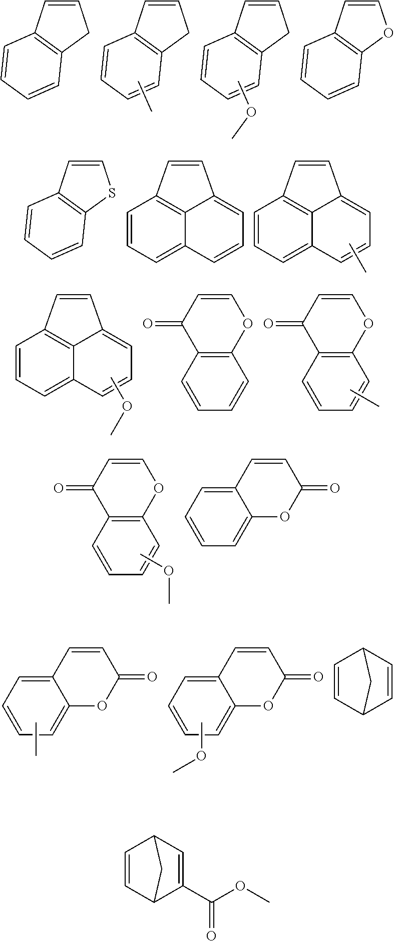

Furthermore, the polymer may comprise repeat units (g) derived from indane, vinylpyridine, or vinylcarbazole.

In the polymer, a fraction of the repeat units (a1), (a2), (b), (c1), (c2), (d1) to (d4), (e), (f), and (g) is: preferably 0<a1≤0.8, 0≤a2≤0.8, 0≤b≤0.5, 0≤c1≤0.6, 0≤c2≤0.6, 0≤d1≤0.4, 0≤d2≤0.4, 0≤d3≤0.4, 0≤d4≤0.4, 0≤e≤0.4, 0≤f≤0.3, and 0≤g≤0.3; more preferably 0<a1≤0.7, 0≤a2≤0.7, 0≤b≤0.3, 0≤c1≤0.5, 0≤c2≤0.5, 0≤d1≤0.3, 0≤d2≤0.3, 0≤d3≤0.3, 0≤d4≤0.3, 0≤e≤0.3, 0≤f≤0.3, and 0≤g≤0.3, provided that a1+a2+b+c1+c2+d1+d2+d3+d4+e+f+g≤1.

The polymer should preferably have a weight average molecular weight (Mw) in the range of 1,000 to 500,000, and more preferably 3,000 to 100,000. A Mw in the range ensures satisfactory etch resistance and eliminates the risk of resolution being lowered due to a failure to acquire a difference in dissolution rate before and after exposure. It is noted that Mw is as measured by gel permeation chromatography (GPC) versus polystyrene standards using tetrahydrofuran (THF) or N,N-dimethylformamide (DMF) solvent.

Since the influence of dispersity (Mw/Mn) becomes stronger as the pattern rule becomes finer, the polymer should preferably have a narrow dispersity (Mw/Mn) of 1.0 to 2.0 in order to provide a resist composition suitable for micropatterning to a small feature size. A Mw/Mn in the range indicates smaller amounts of lower and higher molecular weight fractions and eliminates the risk of leaving foreign matter on the pattern or degrading the pattern profile after exposure and development.

The polymer may be synthesized by any desired methods, for example, by dissolving one or more monomers selected from the monomers corresponding to the foregoing repeat units in an organic solvent, adding a radical polymerization initiator thereto, and heating for polymerization.

Examples of the organic solvent which can be used for polymerization include toluene, benzene, THF, diethyl ether, dioxane, cyclohexane, cyclopentane, methyl ethyl ketone (MEK), propylene glycol monomethyl ether acetate (PGMEA), and γ-butyrolactone (GBL). Examples of the polymerization initiator used herein include 2,2′-azobisisobutyronitrile (AIBN), 2,2′-azobis(2,4-dimethylvaleronitrile), dimethyl-2,2-azobis(2-methylpropionate), 1,1′-azobis(1-acetoxy-1-phenylethane), benzoyl peroxide, and lauroyl peroxide. The amount of the initiator added is preferably 0.01 to 25 mol % based on the total of monomers. The reaction temperature is preferably 50 to 150° C., more preferably 60 to 100° C. The reaction time is preferably 2 to 24 hours, a time of 2 to 12 hours being more preferred in view of production efficiency.

The polymerization initiator may be added to the monomer solution, which is fed to the reactor. Alternatively, a solution of the polymerization initiator is prepared separately from the monomer solution, and the monomer and initiator solutions may be independently fed to the reactor. Since there is a possibility that the initiator generates a radical in the standby time, by which polymerization reaction takes place to form an ultrahigh molecular weight compound, it is preferred from the standpoint of quality control that the monomer solution and the initiator solution be independently prepared and added dropwise. The acid labile group that has been incorporated in the monomer may be kept as such, or the polymerization may be followed by protection or partial protection. Any of well-known chain transfer agents such as dodecylmercaptan and 2-mercaptoethanol may be used for the purpose of adjusting molecular weight. An appropriate amount of the chain transfer agent is 0.01 to 20 mol % based on the total of monomers to be polymerized.

Where a monomer having a hydroxy group is copolymerized, the hydroxy group may be substituted by an acetal group susceptible to deprotection with acid, typically ethoxyethoxy, prior to polymerization, and the polymerization be followed by deprotection with weak acid and water. Alternatively, the hydroxy group may be substituted by an acetyl, formyl, pivaloyl or similar group prior to polymerization, and the polymerization be followed by alkaline hydrolysis.

Hydroxystyrene or hydroxyvinylnaphthalene may be copolymerized by combining it with another monomer in an organic solvent, adding a radical polymerization initiator, and heating the mixture for polymerization. Alternatively, acetoxystyrene or acetoxyvinylnaphthalene is used instead of hydroxystyrene or hydroxyvinylnaphthalene, and after polymerization, the acetoxy group is deprotected by alkaline hydrolysis, for thereby converting the polymer product to hydroxystyrene or hydroxyvinylnaphthalene.

For alkaline hydrolysis, a base such as aqueous ammonia or triethylamine may be used. The reaction temperature is preferably −20 to 100° C., more preferably 0 to 60° C. The reaction time is preferably 0.2 to 100 hours, more preferably 0.5 to 20 hours.

The amounts of monomers in the monomer solution may be determined appropriate so as to provide the preferred fractions of repeat units as mentioned above.

It is described how to use the polymer obtained by the above preparation method. The reaction solution resulting from polymerization reaction may be used as the final product. Alternatively, the polymer may be recovered in powder form through a purifying step such as re-precipitation step of adding the reaction solution to a poor solvent and letting the polymer precipitate as powder, after which the polymer powder is used as the final product. It is preferred from the standpoints of operation efficiency and consistent quality to handle a polymer solution which is obtained by dissolving the powder polymer resulting from the purifying step in a solvent, as the final product.

The solvents which can be used herein are described in JP-A 2008-111103, paragraphs [0144]-[0145](U.S. Pat. No. 7,537,880). Examples of the solvents include ketones such as cyclohexanone and methyl-2-n-pentyl ketone; alcohols such as 3-methoxybutanol, 3-methyl-3-methoxybutanol, 1-methoxy-2-propanol, and 1-ethoxy-2-propanol; ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as GBL; alcohols such as diacetone alcohol (DAA); and high-boiling alcohols such as diethylene glycol, propylene glycol, glycerol, 1,4-butanediol, and 1,3-butanediol, which may be used alone or in admixture.

The polymer solution preferably has a polymer concentration of 0.01 to 30 wt %, more preferably 0.1 to 20 wt %.

Prior to use, the reaction solution or polymer solution is preferably filtered through a filter. Filtration is effective for consistent quality because foreign matter and gel which can cause defects are removed.

Suitable materials of which the filter is made include fluorocarbon, cellulose, nylon, polyester, and hydrocarbon base materials. Preferred for the filtration of a resist composition are filters made of fluorocarbons commonly known as Teflon®, hydrocarbons such as polyethylene and polypropylene, and nylon. While the pore size of the filter may be selected appropriate to comply with the desired cleanness, the filter preferably has a pore size of up to 100 nm, more preferably up to 20 nm. A single filter may be used or a plurality of filters may be used in combination. Although the filtering method may be single pass of the solution, preferably the filtering step is repeated by flowing the solution in a circulating manner. In the polymer preparation process, the filtering step may be carried out any times, in any order and in any stage. The reaction solution as polymerized or the polymer solution may be filtered, preferably both are filtered.

The base polymer (A) may be used alone or as a mixture of two or more polymers which are different in compositional ratio, Mw and/or Mw/Mn. In addition to the polymer defined above, the base polymer (A) may contain a hydrogenated product of ring-opening metathesis polymerization (ROMP) polymer, which is described in JP-A 2003-066612.

The base polymer (A) of the invention preferably comprises the repeat units having a photoacid generating group particularly when narrow pitch patterns are formed by applying the EUV lithography. It is more preferred that an acid generating anion structure be bound to the polymer. This minimizes acid diffusion. Further preferably, the base polymer (A) of the invention additionally contains an aromatic moiety in the repeat unit having an acid labile group and comprises phenolic hydroxy group-containing units having formula (c2). Incorporation of aromatic-bearing groups ensures improvements in etch resistance and the efficiency of generating secondary electrons upon exposure to EUV radiation.

### (B) Organic Solvent

The resist composition of the invention comprises an organic solvent as component (B). The organic solvent used herein is not particularly limited as long as the foregoing component (A) and other components are soluble therein. Examples of the organic solvent include ketones such as cyclohexanone, cyclopentanone, methyl-2-n-pentyl ketone, and 2-heptanone; alcohols such as 3-methoxybutanol, 3-methyl-3-methoxybutanol, 1-methoxy-2-propanol, 1-ethoxy-2-propanol, and DAA; ethers such as PGME, ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, methyl 2-hydroxyisobutyrate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones such as GBL, as described in JP-A 2008-111103, paragraphs [0144]-[0145](U.S. Pat. No. 7,537,880).

In the resist composition of the invention, the organic solvent (B) is preferably added in an amount of 100 to 10,000 parts by weight, and more preferably 200 to 8,000 parts by weight per 80 parts by weight of the base polymer (A). The organic solvent (B) may be used alone or in admixture of two or more.

### (C) Hypervalent Iodine Compound

The resist composition of the invention further comprises a hypervalent iodine compound as component (C) having formula (1).

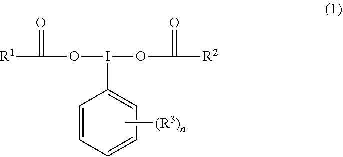

In formula (1), n is an integer of 0 to 5.

In formula (1), R¹ and R² are each independently halogen or a C₁-C₁₀ hydrocarbyl group which may contain a heteroatom. R¹ and R² may bond together to form a ring with the carbon atoms to which they are attached and the intervenient atoms. Examples of the halogen include fluorine, chlorine, bromine and iodine. The C₁-C₁₀ hydrocarbyl group may be saturated or unsaturated and straight, branched, or cyclic. Examples thereof include C₁-C₁₀ alkyl groups such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl, and n-decyl; C₃-C₁₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}]decanyl, and adamantyl; alkenyl groups such as vinyl and allyl; C₆-C₁₀ aryl groups such as phenyl and naphthyl; and combinations thereof. In the foregoing hydrocarbyl groups, some or all of the hydrogen atoms may be substituted by a moiety containing a heteroatom such as oxygen, sulfur, nitrogen or halogen, or some constituent —CH₂— may be substituted by a moiety containing a heteroatom such as oxygen, sulfur or nitrogen, so that the group may contain a hydroxy, cyano, halogen, carbonyl, ether bond, thioether bond, ester bond, sulfonic ester bond, carbonate bond, carbamate bond, lactone ring, sultone ring or carboxylic anhydride (—C(═O)—O—C(═O)—). R¹ and R² are preferably C₁-C₄ hydrocarbyl groups.

In formula (1), R³ is halogen or a C₁-C₄₀ hydrocarbyl group which may contain a heteroatom. When n is an integer of 2 to 5, a plurality of R³ may be the same or different. Examples of the halogen include fluorine, chlorine, bromine and iodine. The C₁-C₄₀ hydrocarbyl group may be saturated or unsaturated and straight, branched, or cyclic. Examples thereof include C₁-C₄₀ alkyl groups such as methyl, ethyl, propyl, isopropyl, n-butyl, sec-butyl, tert-butyl, tert-pentyl, n-pentyl, n-hexyl, n-octyl, 2-ethylhexyl, n-nonyl, and n-decyl; C₃-C₄₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}]decanyl, adamantyl, and adamantylmethyl; and C₆-C₄₀ aryl groups such as phenyl, naphthyl, and anthracenyl. In the foregoing hydrocarbyl groups, some or all of the hydrogen atoms may be substituted by a moiety containing a heteroatom such as oxygen, sulfur, nitrogen or halogen, or some constituent —CH₂— may be substituted by a moiety containing a heteroatom such as oxygen, sulfur or nitrogen, so that the group may contain a hydroxy, cyano, halogen, carbonyl, ether bond, thioether bond, ester bond, sulfonic ester bond, carbonate bond, carbamate bond, lactone ring, sultone ring or carboxylic anhydride (—C(═O)—O—C(═O)—).

Examples of the hypervalent iodine compound having formula (1) are shown below, but not limited thereto.

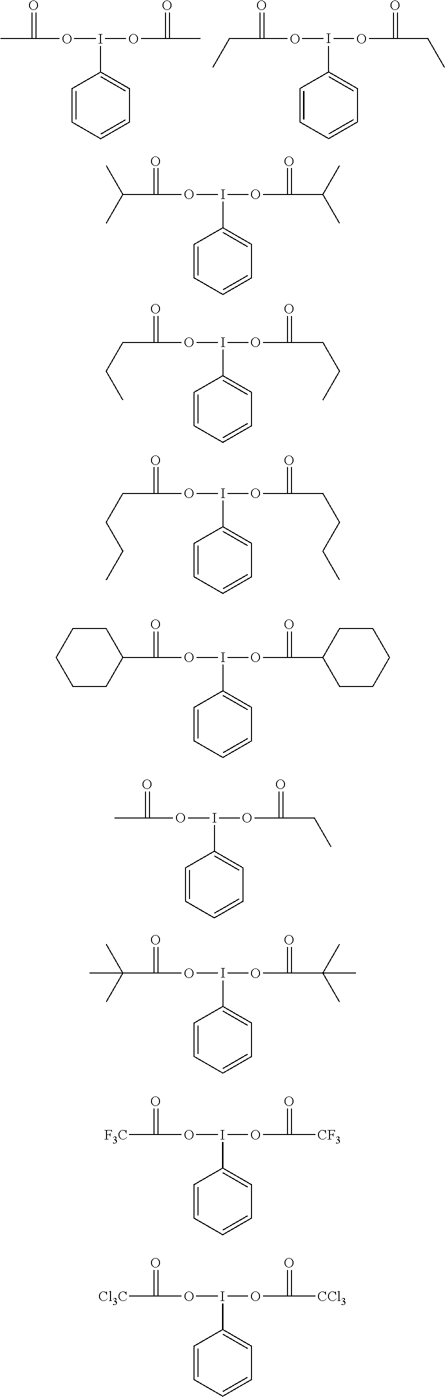

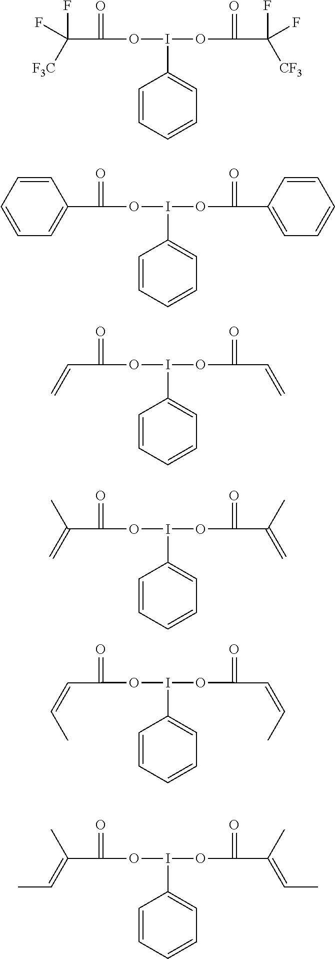

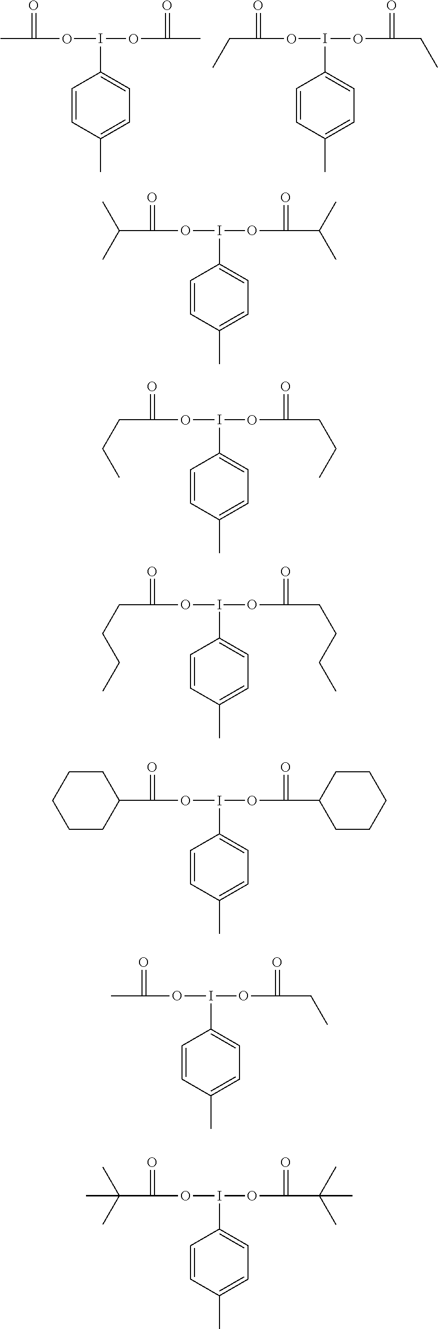

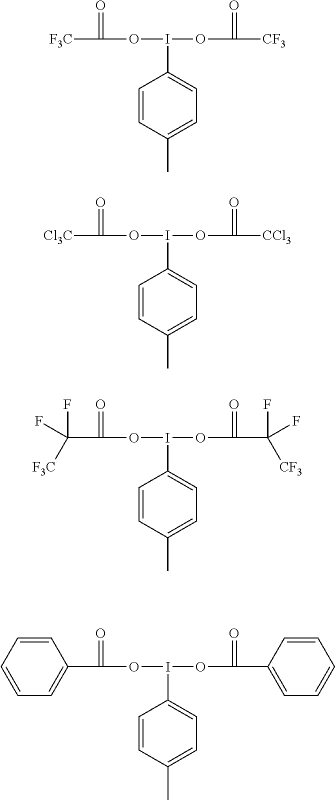

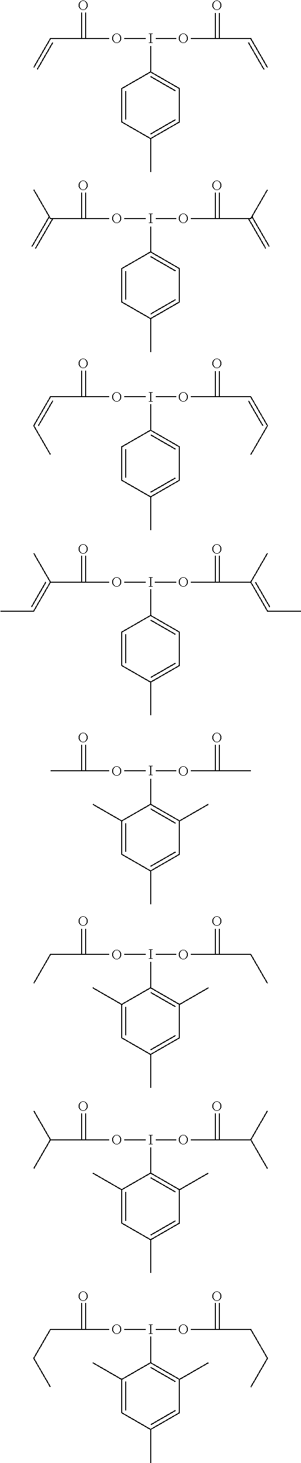

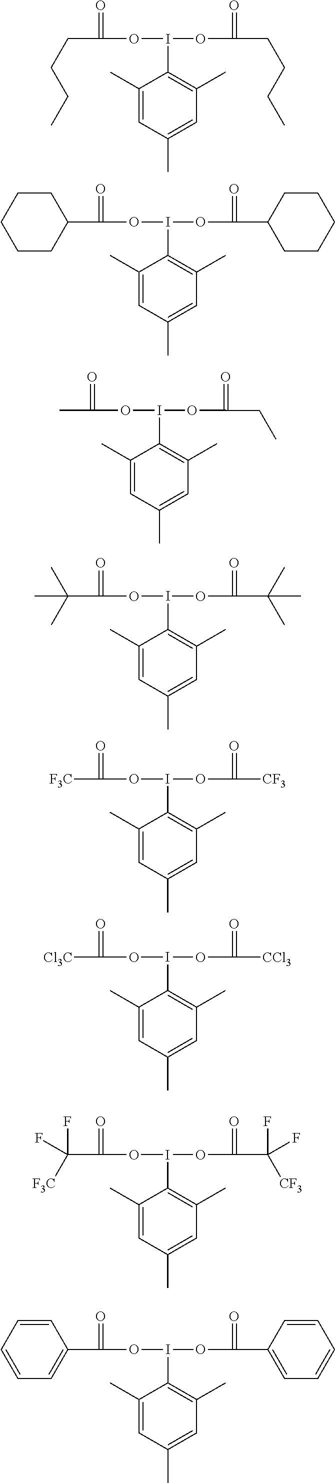

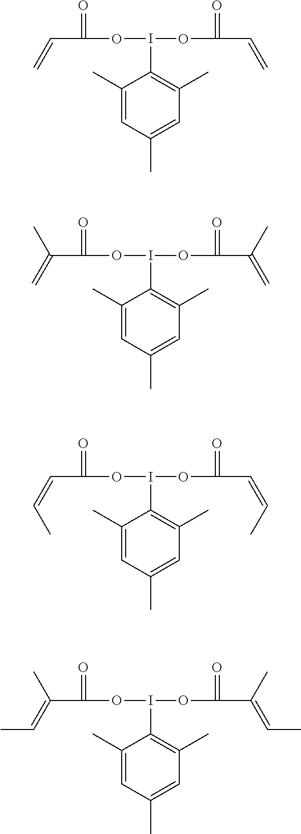

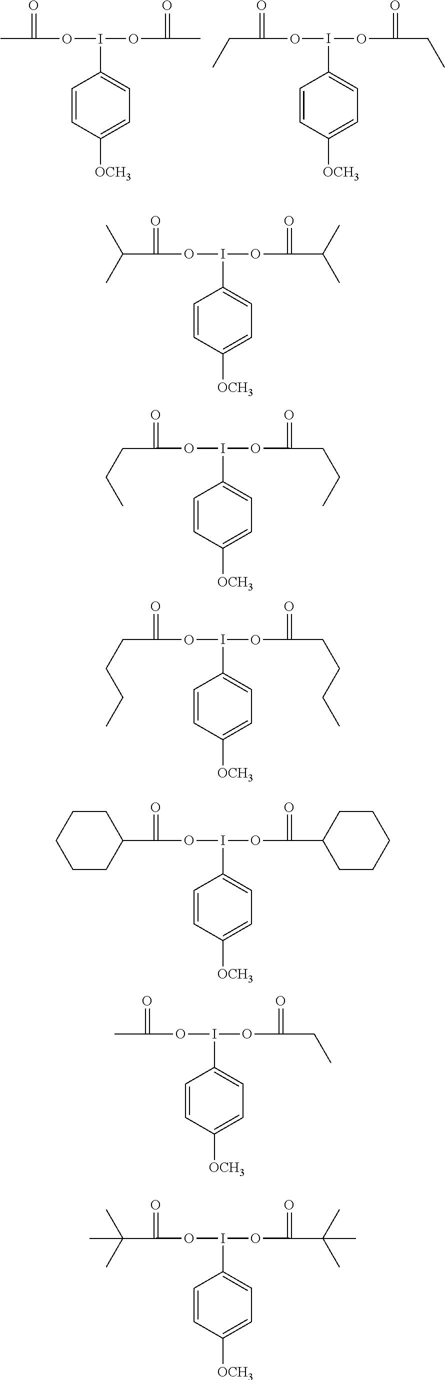

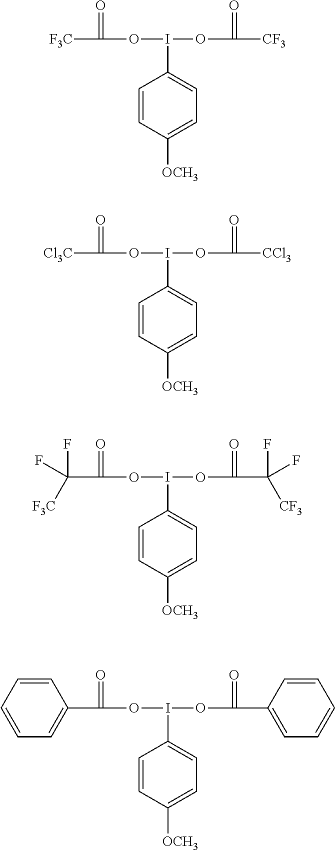

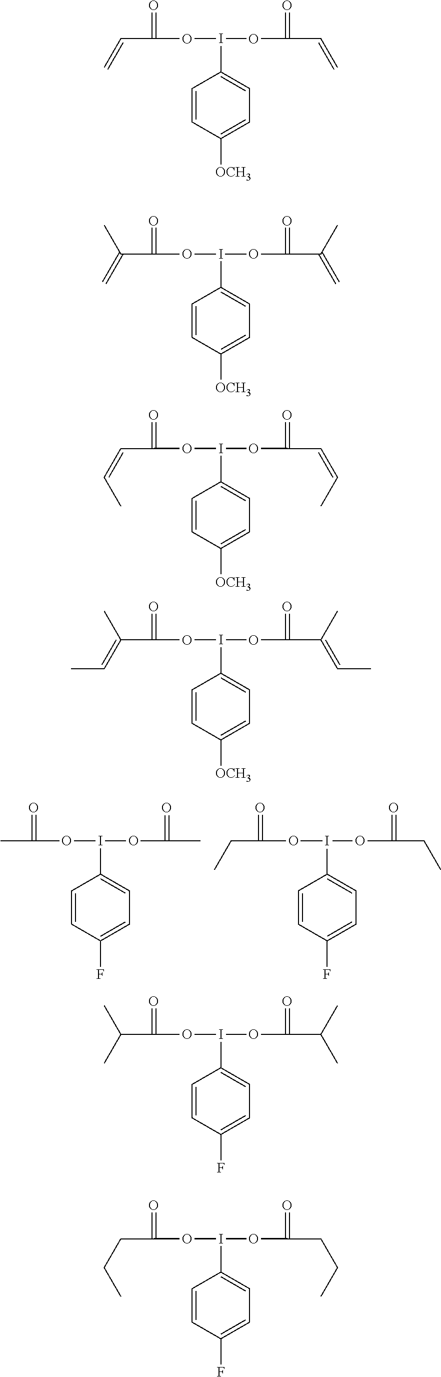

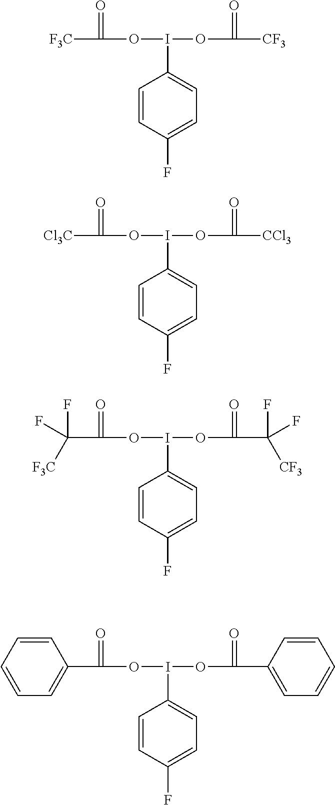

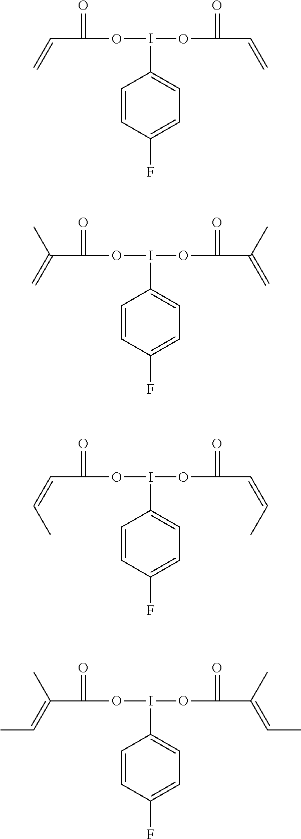

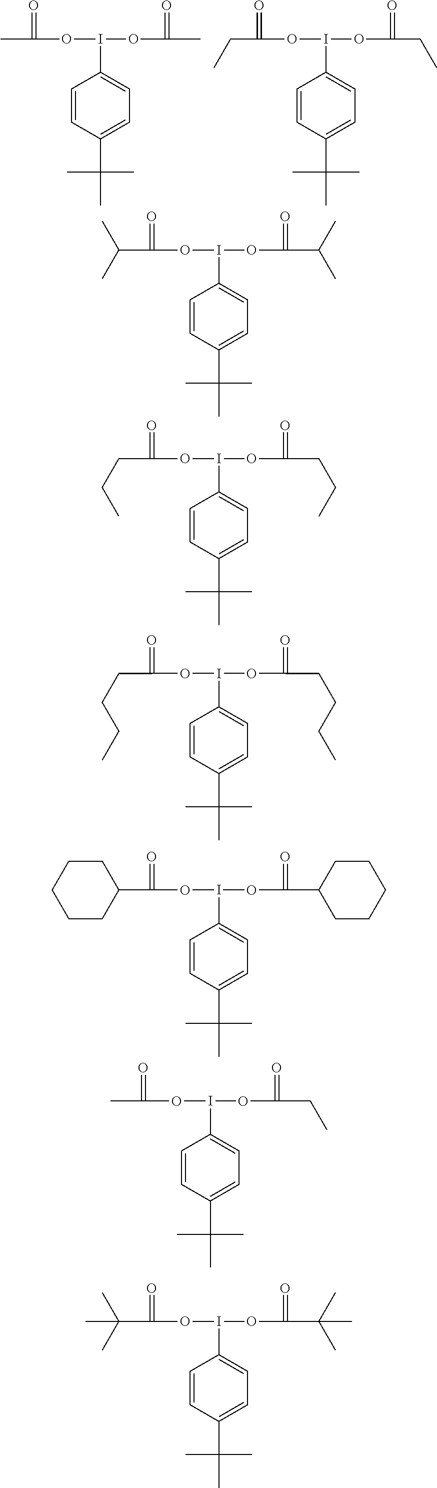

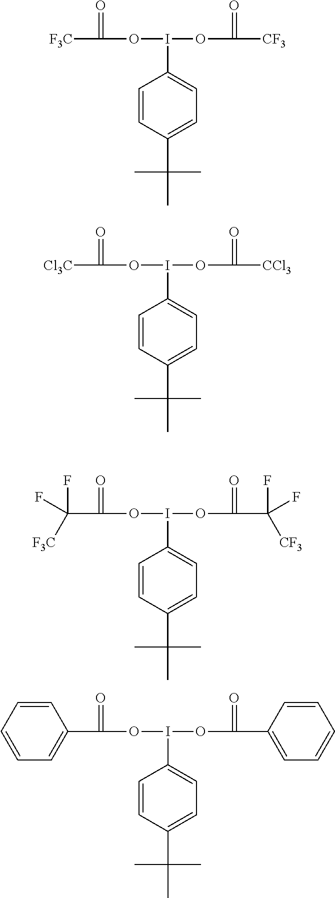

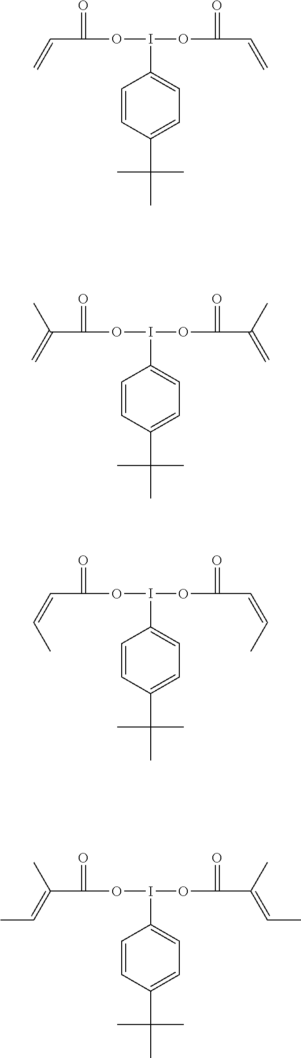

The hypervalent iodine compound having formula (1) is preferably added in an amount of 1 to 50 wt %, and more preferably 2 to 30 wt % based on the overall solids in the resist composition. As long as the amount is in the range, sufficient sensitivity and resolution are achieved, and the risk of foreign matter and defects appearing during film formation is avoided.

The resist composition of the invention essentially comprises (A) the polymer comprising the repeat units having an acid labile group and the repeat units having a carboxy group, (B) the organic solvent, and (C) the hypervalent iodine compound having formula (1). This makes it possible to form a high-contrast image in fine pattern formation, to thereby improve maximum resolution and LWR. Although its reason is not well understood, the following reason is presumed.

The resist composition of the invention is a so-called chemically amplified resist composition in which the acid labile group in the polymer is cleaved by the acid generated by exposure from a photoacid generator to create a polar group and exhibit dissolution contrast between exposed and unexposed regions. In addition to this typical reaction mechanism of the chemically amplified resist composition, another reaction is considered to proceed in the invention. The reaction is a reaction related to the carboxy group in the polymer and the hypervalent iodine compound having formula (1).

The hypervalent iodine compound having formula (1) is a three-coordinate iodine compound. The iodine-oxygen bond in the hypervalent iodine compound is a relatively weak bond called a 3-center 4-electron bond, and with a carboxy group-containing compound outside the bond, a ligand exchange reaction is considered to proceed in equilibrium. In view of this, in the resist composition of the invention, the hypervalent iodine compound is partially bonded to the carboxy group in the polymer during film formation. It is presumed that the polymers are crosslinked by the hypervalent iodine compound.

Since the iodine-oxygen bond of the hypervalent iodine compound is weak as mentioned above, the bond is cleaved by exposure. The polymers crosslinked by the hypervalent iodine compound during film formation is cleaved by exposure, so that the molecular weight is greatly reduced in the exposed region.

Based on the above presumption, the resist composition of the invention exhibits dissolution contrast by two factors, that is, a polarity change and a molecular weight change. Therefore, a higher contrast than that in a conventional chemically amplified resist composition can be obtained, and resolution is improved.

Patent Document 3 discloses a resist composition to which a hypervalent iodine compound is added. However, Patent Document 3 describes that the resist composition is successful only in improving line edge roughness, and refers nowhere to a necessity of a carboxy group-containing unit for a polymer to be applied. It would be difficult for the resist composition described in Patent Document 3 to improve resolution by increasing contrast.

Patent Document 4 discloses application of a polymer for resist prepared using a hypervalent iodine compound containing a polymerizable group. The hypervalent iodine compound of Patent Document 4 crosslinks polymers, but in this case, the crosslinking density is too high, the solubility of the developer after exposure is deteriorated, and a residue is generated due to insoluble matter during development, causing degradations to resolution and LWR. On the other hand, in the resist composition of the invention, since the partial crosslinking is performed during film formation, the exposed region is easily dissolved in the developer, and the resolution is improved. The idea of improving the resolution by the partial crosslinking in the ligand exchange reaction during film formation cannot be obtained from Patent Document 4, and therefore it can be said that the resist composition of the invention can solve such a problem by a novel proposal.

### (D) Photoacid Generator

The resist composition of the invention may comprise a photoacid generator as component (D). The photoacid generator as component (D) is not particularly limited as long as it is capable of generating an acid upon exposure to high-energy radiation. The preferred photoacid generator is a salt having formula (2).

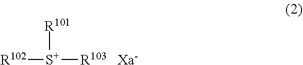

In formula (2), R¹⁰¹, R¹⁰² and R¹⁰³ are each independently a C₁-C₂₀ hydrocarbyl group which may contain a heteroatom. Any two of R¹⁰¹, R¹⁰² and R¹⁰³ may bond together to form a ring with the sulfur atom to which they are attached. The hydrocarbyl group may be saturated or unsaturated and straight, branched or cyclic. Examples thereof are as exemplified above for the hydrocarbyl groups R³¹ and R³² in formula (d1). Examples of the cation in the sulfonium salt having formula (2) are as will be exemplified later for the sulfonium cation having formula (6-1) which is described later.

In formula (2), Xa⁻ is a non-nucleophilic counter anion. Examples of the non-nucleophilic counter anion include anions selected from formulae (2A) to (2D).

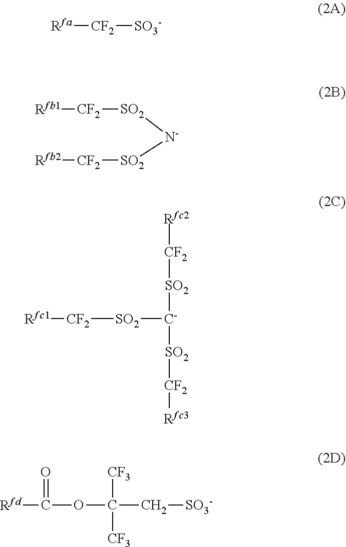

In formula (2A), R^{fa} is fluorine or a C₁-C₄₀ hydrocarbyl group which may contain a heteroatom. The hydrocarbyl group may be saturated or unsaturated and straight, branched or cyclic. Examples thereof are as will be exemplified later for the hydrocarbyl group R^{fa1} in formula (2A′).

Of the anions having formula (2A), anions having formula (2A′) are preferred.

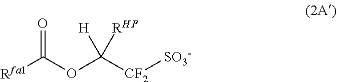

In formula (2A′), R^{HF} is hydrogen or trifluoromethyl, preferably trifluoromethyl.

R^{fa1} is a C₁-C₃₈ hydrocarbyl group which may contain a heteroatom. Suitable heteroatoms include oxygen, nitrogen, sulfur, and halogen atoms, with oxygen being preferred. Of the hydrocarbyl groups, those of 6 to 30 carbon atoms are preferred because a high resolution is available in fine pattern formation.

The C₁-C₃₈ hydrocarbyl group represented by R^{fa1} may be saturated or unsaturated and straight, branched or cyclic. Examples thereof include C₁-C₃₀ alkyl groups such as methyl, ethyl, n-propyl, isopropyl, n-butyl, isobutyl, sec-butyl, tert-butyl, pentyl, neopentyl, hexyl, heptyl, octyl, 2-ethylhexyl, nonyl, decyl, undecyl, dodecyl, tridecyl, pentadecyl, heptadecyl, and icosyl; C₃-C₃₀ cyclic saturated hydrocarbyl groups such as cyclopentyl, cyclohexyl, 1-adamantyl, 2-adamantyl, 1-adamantylmethyl, norbornyl, norbornylmethyl, tricyclodecanyl, tetracyclododecanyl, tetracyclododecanylmethyl, and dicyclohexylmethyl; C₂-C₃₀ unsaturated aliphatic hydrocarbyl groups such as allyl and 3-cyclohexenyl; C₆-C₃₀ aryl groups such as phenyl, 1-naphthyl and 2-naphthyl; C₇-C₃₈ aralkyl groups such as benzyl and diphenylmethyl; and combinations thereof.

In the foregoing hydrocarbyl groups, some or all of the hydrogen atoms may be substituted by a moiety containing a heteroatom such as oxygen, sulfur, nitrogen or halogen, or some constituent —CH₂— may be substituted by a moiety containing a heteroatom such as oxygen, sulfur or nitrogen, so that the group may contain a hydroxy, fluorine, chlorine, bromine, iodine, cyano, carbonyl, ether bond, ester bond, sulfonic ester bond, carbonate bond, lactone ring, sultone ring, carboxylic anhydride (—C(═O)—O—C(═O)—) or haloalkyl moiety. Examples of the heteroatom-containing hydrocarbyl group include tetrahydrofuryl, methoxymethyl, ethoxymethyl, methylthiomethyl, acetamidomethyl, trifluoroethyl, (2-methoxyethoxy)methyl, acetoxymethyl, 2-carboxy-1-cyclohexyl, 2-oxopropyl, 4-oxo-1-adamantyl, 5-hydroxy-1-adamantyl, 5-tert-butylcarbonyloxy-1-adamantyl, 4-oxatricyclo[4.2.1.0^{3,7}]nonan-5-on-2-yl, and 3-oxocyclohexyl.

With respect to the synthesis of the sulfonium salt having an anion of formula (2A′), reference may be made to JP-A 2007-145797, JP-A 2008-106045, JP-A 2009-007327, and JP-A 2009-258695. Also useful are the sulfonium salts described in JP-A 2010-215608, JP-A 2012-041320, JP-A 2012-106986, and JP-A 2012-153644.

Examples of the anion having formula (2A) are as exemplified above for the sulfonate anion having formula (d1-1) or (d1-2).

In formula (2B), R^{fb1} and R^{fb2} are each independently fluorine or a C₁-C₄₀ hydrocarbyl group which may contain a heteroatom. The hydrocarbyl group may be saturated or unsaturated and straight, branched or cyclic. Examples thereof are as exemplified above for the hydrocarbyl group R^{fa1} in formula (2A′). Preferably R^{fb1} and R^{fb2} are fluorine or C₁-C₄ straight fluorinated alkyl groups. Also, R¹¹ and R¹² may bond together to form a ring with the linkage: —CF₂—SO₂—N⁻—SO₂—CF₂⁻ to which they are attached. It is preferred that a combination of R¹¹ and R¹² be a fluorinated ethylene or fluorinated propylene group.

In formula (2C), R^{fc1}, R^{fc2} and R^{fc3} are each independently fluorine or a C₁-C₄₀ hydrocarbyl group which may contain a heteroatom. The hydrocarbyl group may be saturated or unsaturated and straight, branched or cyclic. Examples thereof are as exemplified above for the hydrocarbyl group R^{fa1} in formula (2A′). Preferably R^{fc1}, R^{fc2} and R^{fc3} are fluorine or C₁-C₄ straight fluorinated alkyl groups. Also, R^{fc1} and R^{fc2} may bond together to form a ring with the linkage: —CF₂—SO₂—C⁻—SO₂—CF₂— to which they are attached. It is preferred that a combination of R^{fc1} and R^{fc2} be a fluorinated ethylene or fluorinated propylene group.

In formula (2D), R^{fd} is a C₁-C₄₀ hydrocarbyl group which may contain a heteroatom. The hydrocarbyl group may be saturated or unsaturated and straight, branched or cyclic. Examples thereof are as exemplified above for the hydrocarbyl group R^{fa1} in formula (2A′).

With respect to the synthesis of the sulfonium salt having an anion of formula (2D), reference may be made to JP-A 2010-215608 and JP-A 2014-133723.

Examples of the anion having formula (2D) are shown below, but not limited thereto.

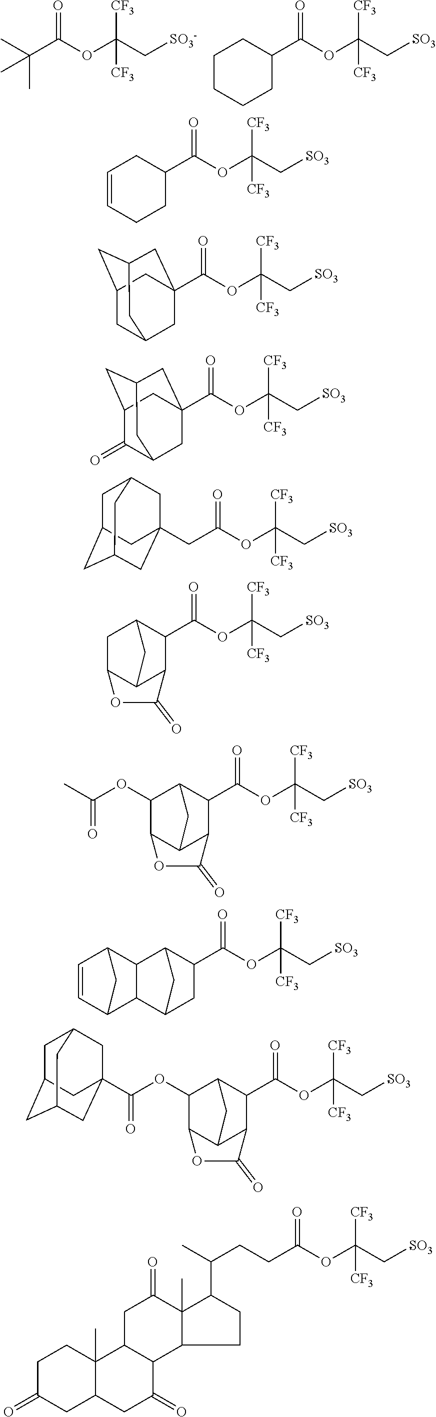

Another example of the non-nucleophilic counter anion is an anion having an iodine or bromine-substituted aromatic ring. The preferred anion has formula (2E).

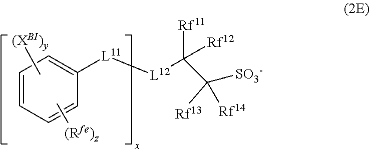

In formula (2E), x is an integer of 1 to 3. y is an integer of 1 to 5, z is an integer of 0 to 3, and y+z is from 1 to 5. y is preferably an integer of 1 to 3, more preferably 2 or 3. z is preferably an integer of 0 to 2.

In formula (2E), X^{BI} is iodine or bromine. When x and/or y is 2 or more, a plurality of X^{BI} may be the same or different.

In formula (2E), L¹¹ is a single bond, ether bond, ester bond, or a C₁-C₆ saturated hydrocarbylene group which may contain an ether bond or ester bond. The saturated hydrocarbylene group may be straight, branched or cyclic.

In formula (2E), L¹² is a single bond or a C₁-C₂₀ divalent linking group when x=1, and a C₁-C₂₀ (x+1)-valent linking group which may contain oxygen, sulfur or nitrogen when x=2 or 3.

In formula (2E), R^{fe} is hydroxy, carboxy, fluorine, chlorine, bromine, amino, or a C₁-C₂₀ hydrocarbyl group, C₁-C₂₀ hydrocarbyloxy group, C₂-C₂₀ hydrocarbylcarbonyl group, C₂-C₂₀ hydrocarbyloxycarbonyl group, C₂-C₂₀ hydrocarbylcarbonyloxy group, or C₁-C₂₀ hydrocarbylsulfonyloxy group, which may contain fluorine, chlorine, bromine, hydroxy, amino or ether bond, or —N(R^{feA})(R^{feB}), —N(R^{feC})—C(═O)—R^{feD} or —N(R^{feC})—C(═O)—O—R^{feD}. R^{feA} and R^{feB} are each independently hydrogen or a C₁-C₆ saturated hydrocarbyl group. R^{feC} is hydrogen or a C₁-C₆ saturated hydrocarbyl group which may contain halogen, hydroxy, C₁-C₆ saturated hydrocarbyloxy, C₂-C₆ saturated hydrocarbylcarbonyl or C₂-C₆ saturated hydrocarbylcarbonyloxy moiety. R^{feD} is a C₁-C₁₆ aliphatic hydrocarbyl group, C₆-C₁₂ aryl group or C₇-C₁₅ aralkyl group, which may contain halogen, hydroxy, C₁-C₆ saturated hydrocarbyloxy, C₂-C₆ saturated hydrocarbylcarbonyl or C₂-C₆ saturated hydrocarbylcarbonyloxy moiety. The aliphatic hydrocarbyl group may be saturated or unsaturated and straight, branched or cyclic. The hydrocarbyl, hydrocarbyloxy, hydrocarbylcarbonyl, hydrocarbyloxycarbonyl, hydrocarbylcarbonyloxy and hydrocarbylsulfonyloxy groups may be straight, branched or cyclic. A plurality of groups R^{fe} may be the same or different when x and/or z is 2 or more.

Of these, R^{fe} is preferably hydroxy, —N(R^{feC})—C(═O)—R^{feD}, —N(R^{feC})—C(═O)—O—R^{feD}, fluorine, chlorine, bromine, methyl or methoxy.

In formula (2E), Rf¹¹ to Rf¹⁴ are each independently hydrogen, fluorine or trifluoromethyl, at least one of Rf¹¹ to Rf¹⁴ is fluorine or trifluoromethyl. Rf¹¹ and Rf¹², taken together, may form a carbonyl group. Preferably, both Rf¹³ and Rf¹⁴ are fluorine.

Examples of the anion having formula (2E) are shown below, but not limited thereto. Herein X^{BI} is as defined above.

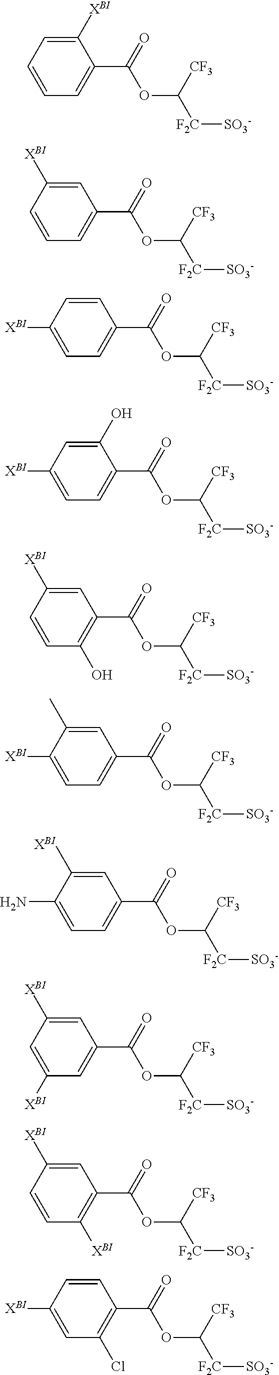

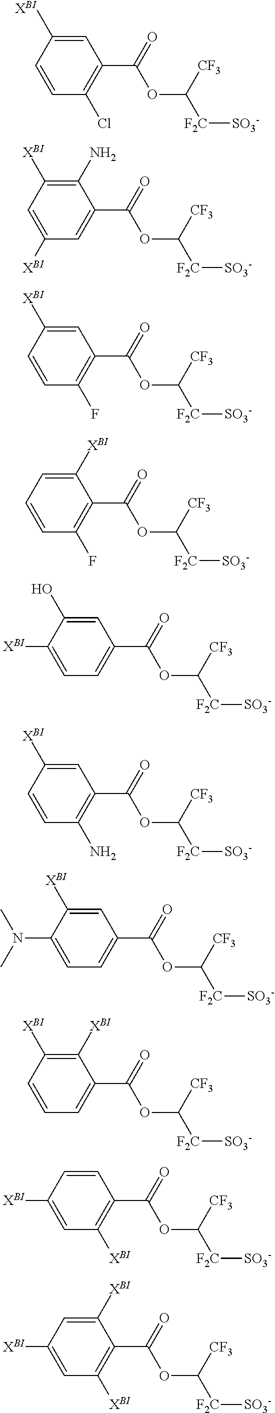

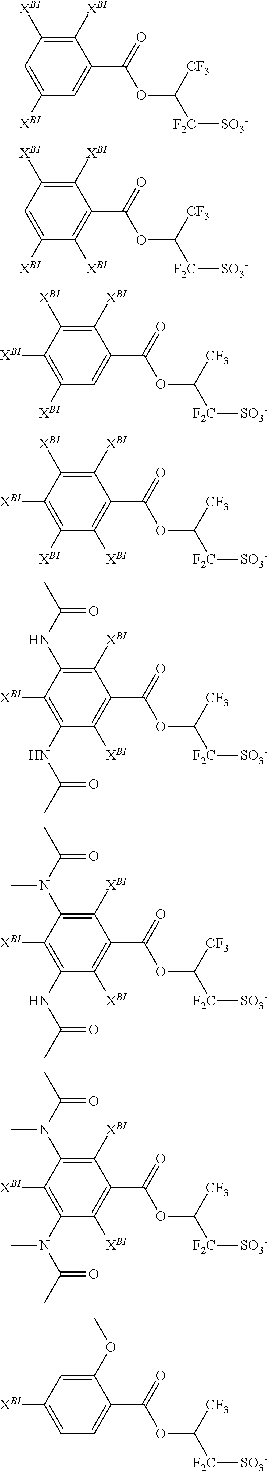

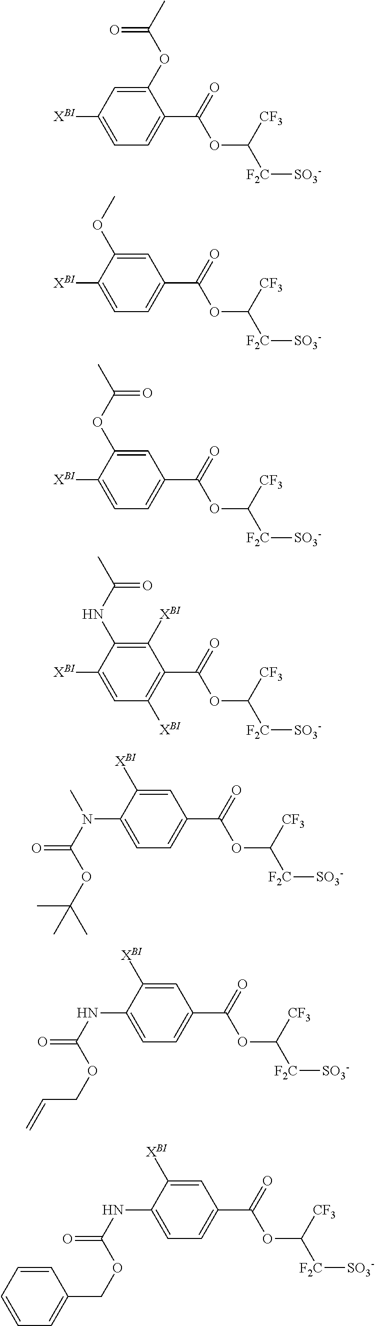

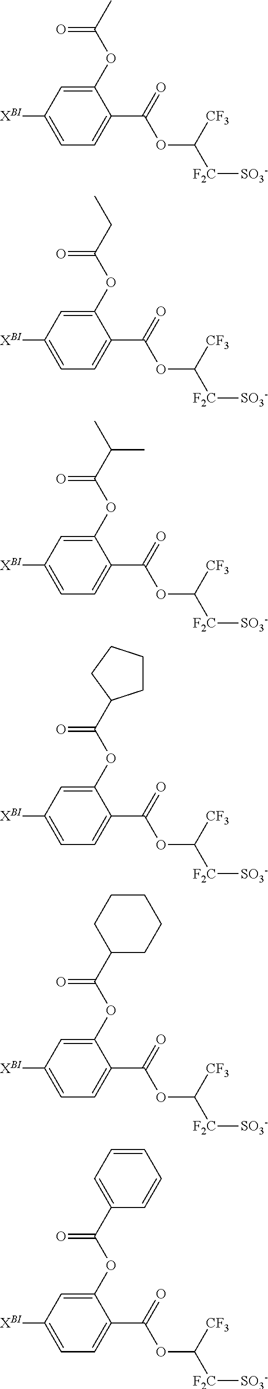

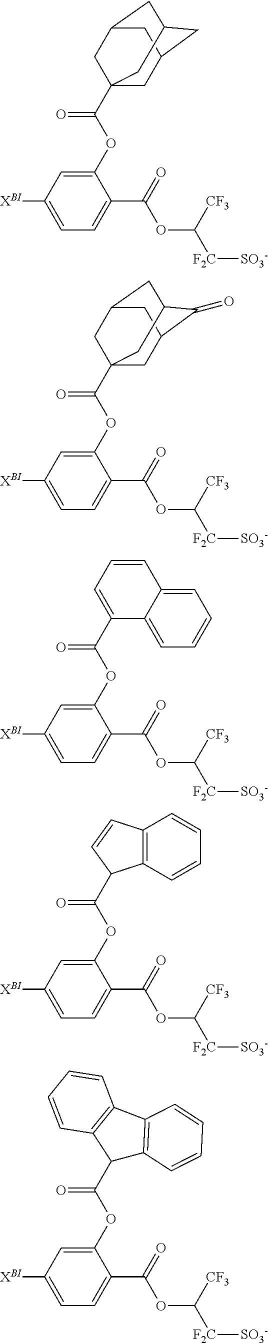

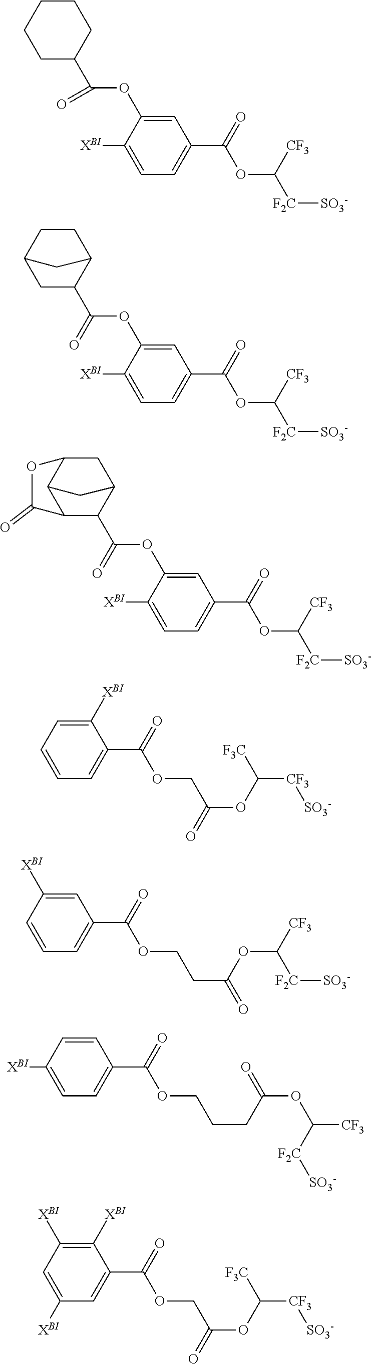

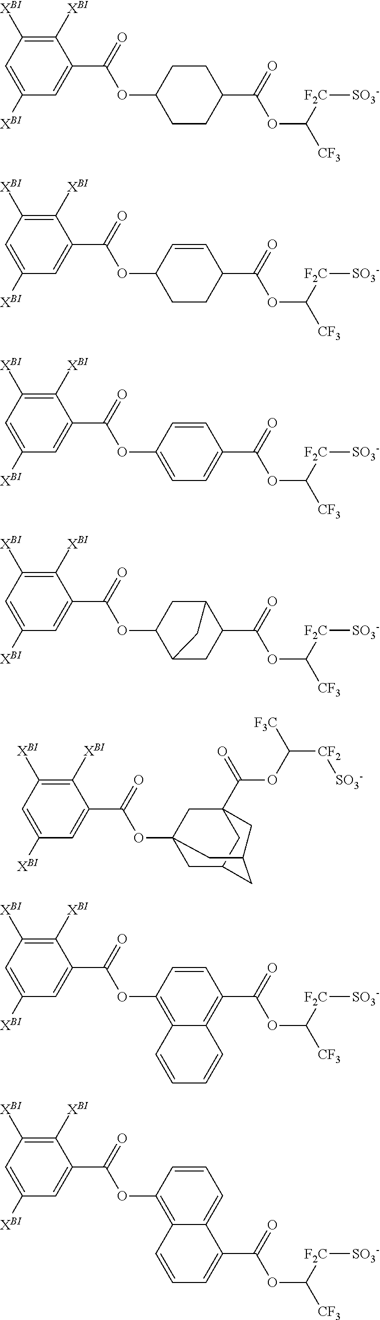

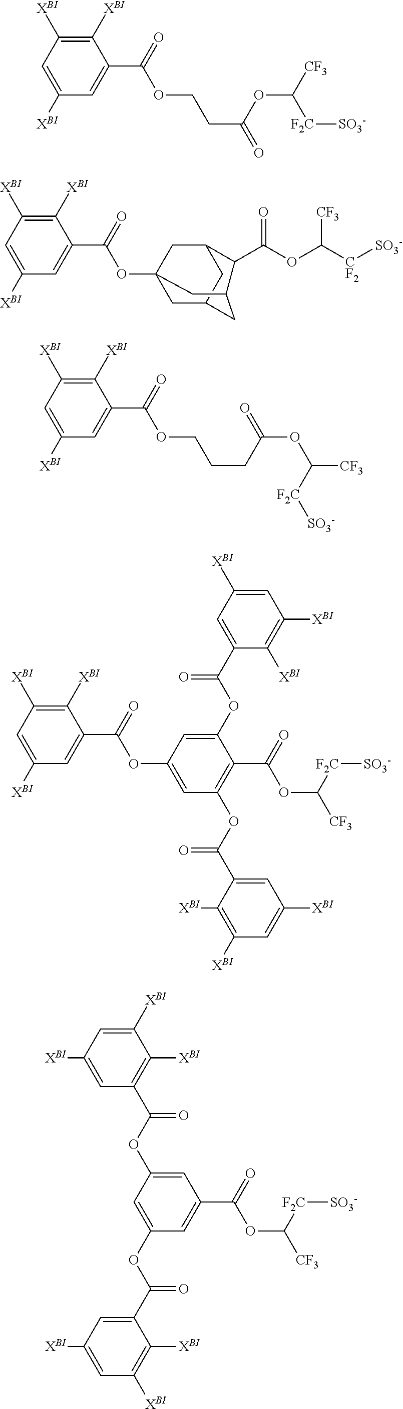

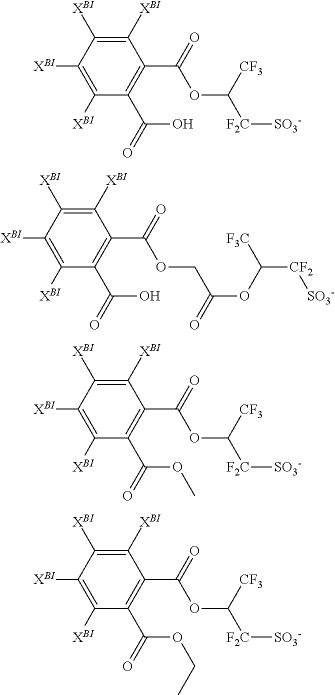

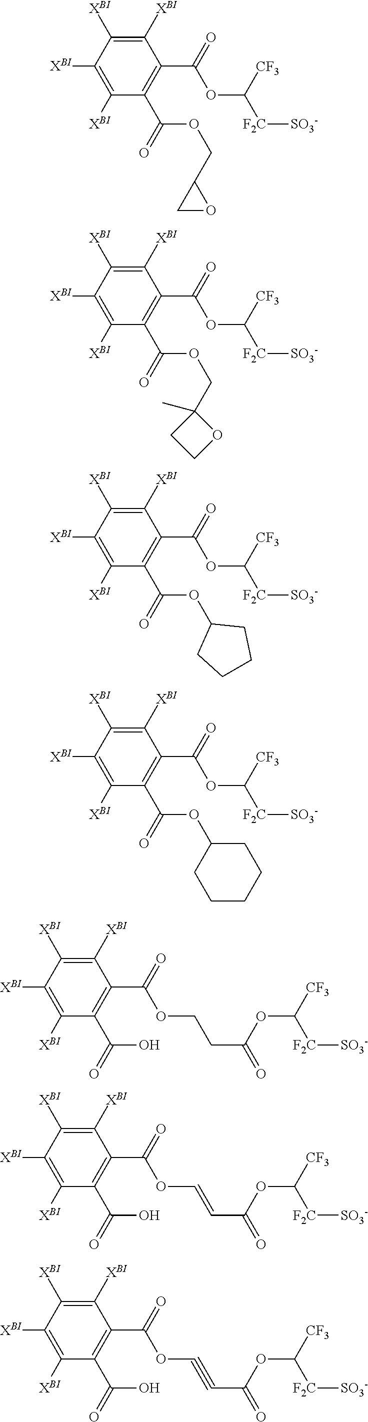

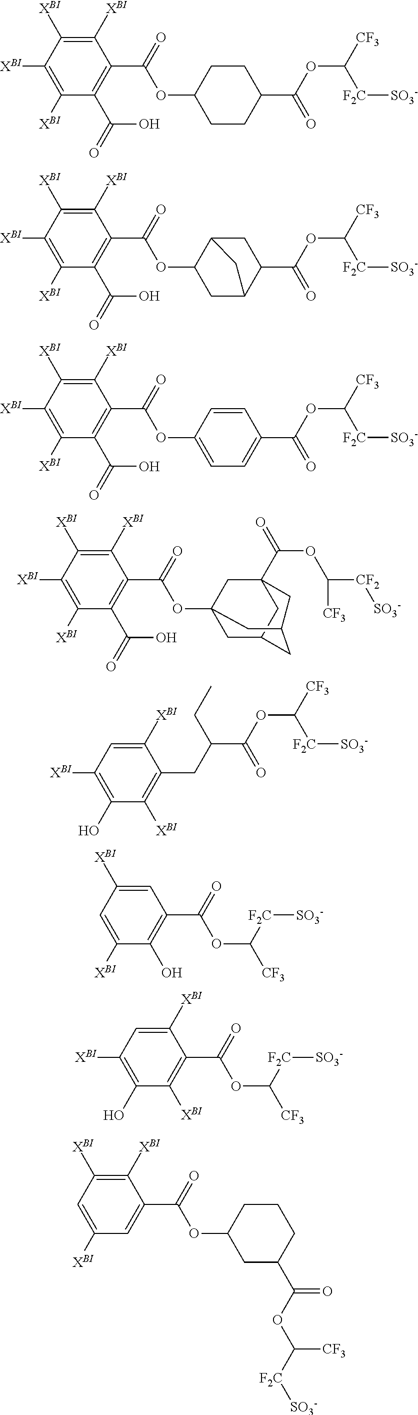

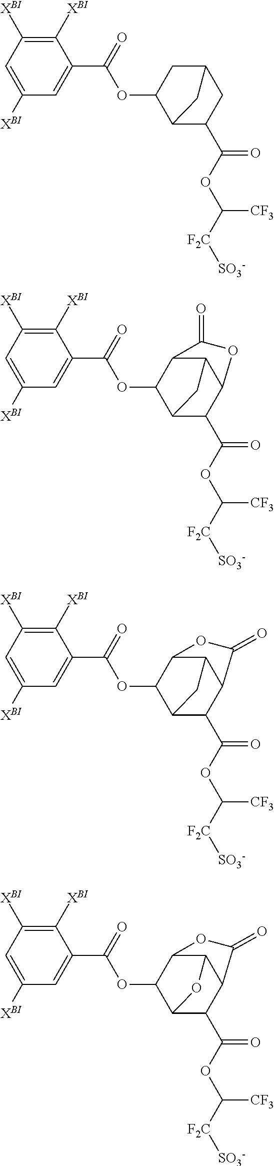

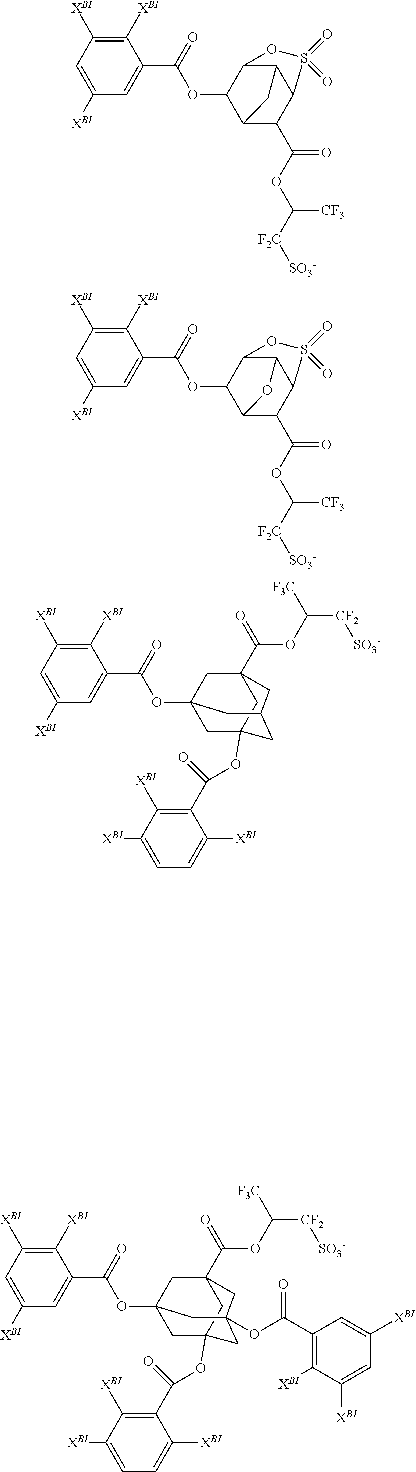

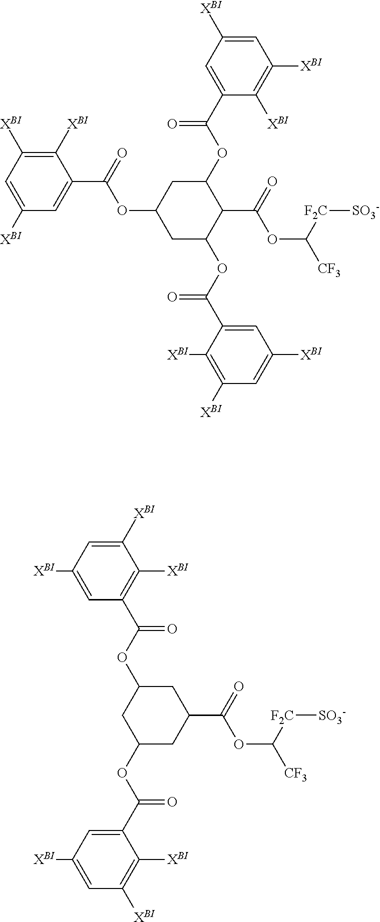

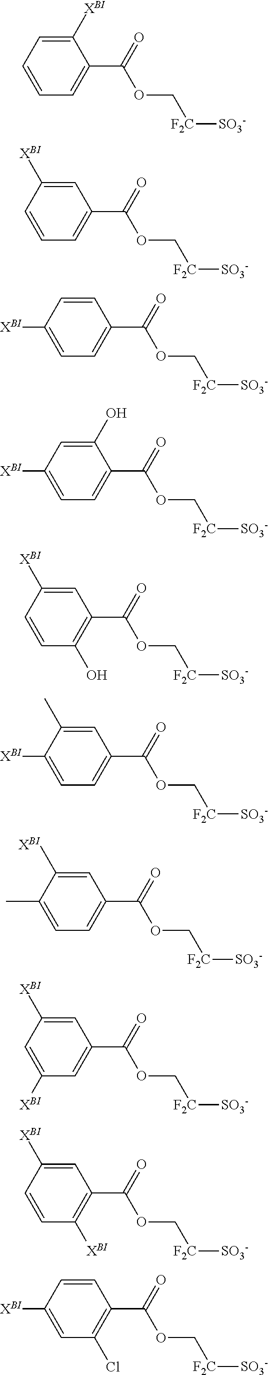

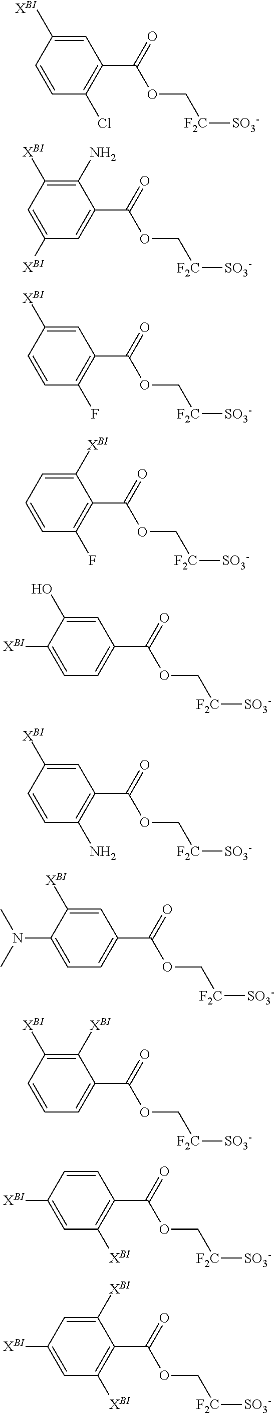

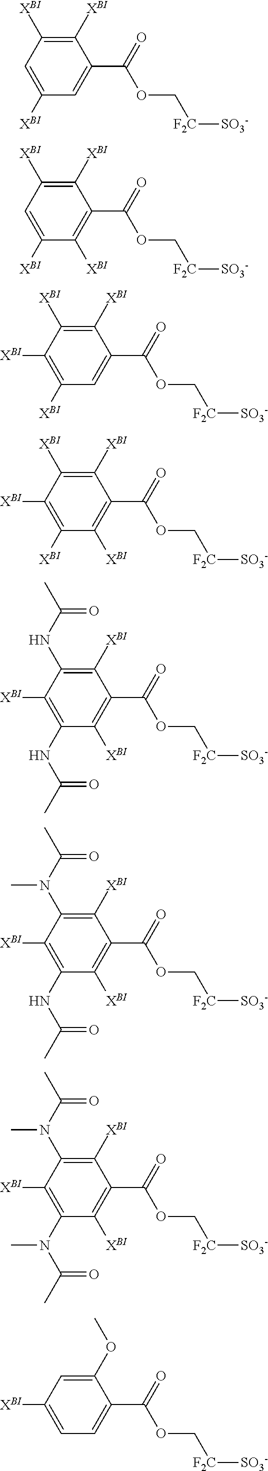

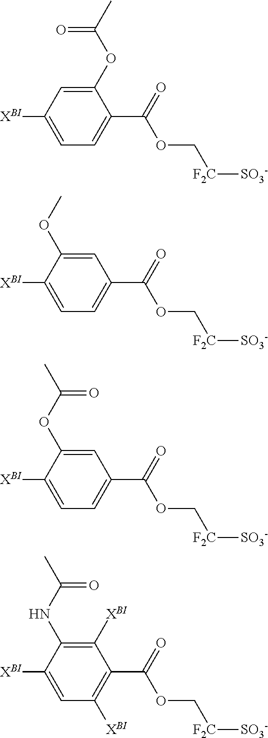

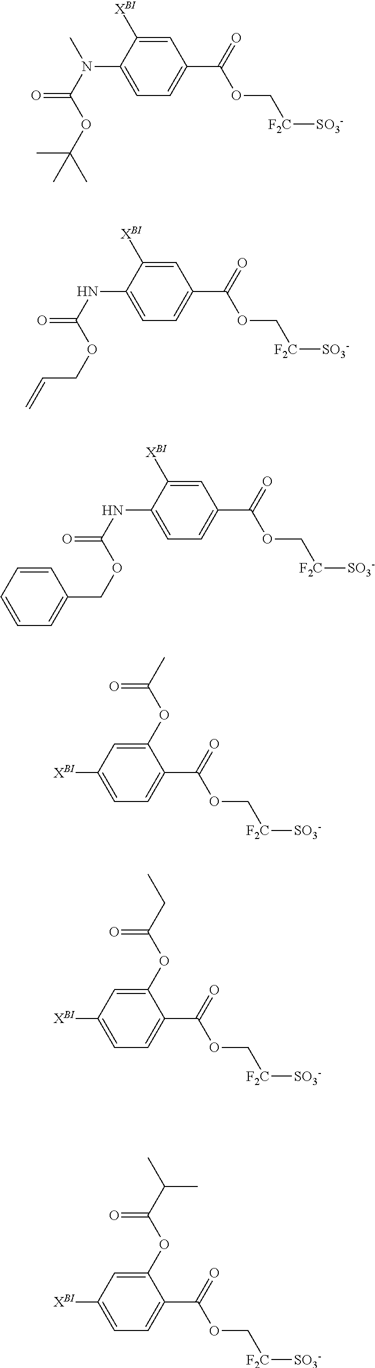

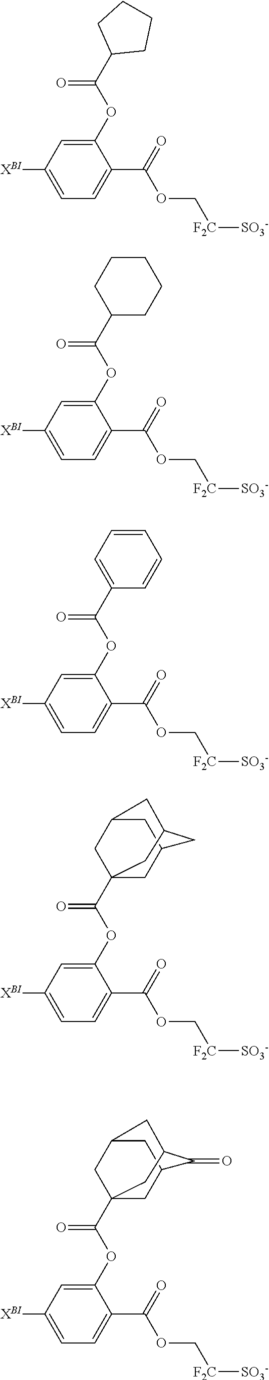

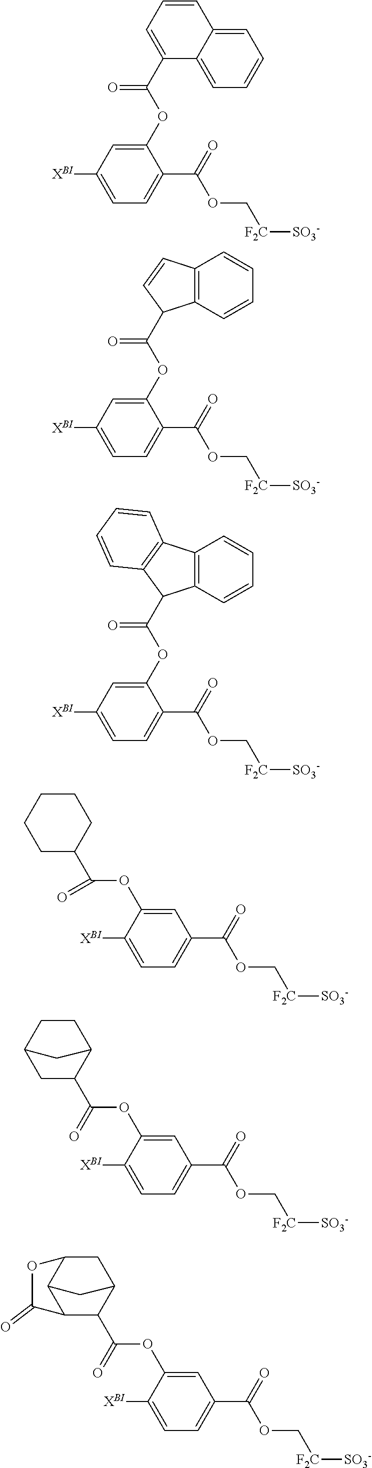

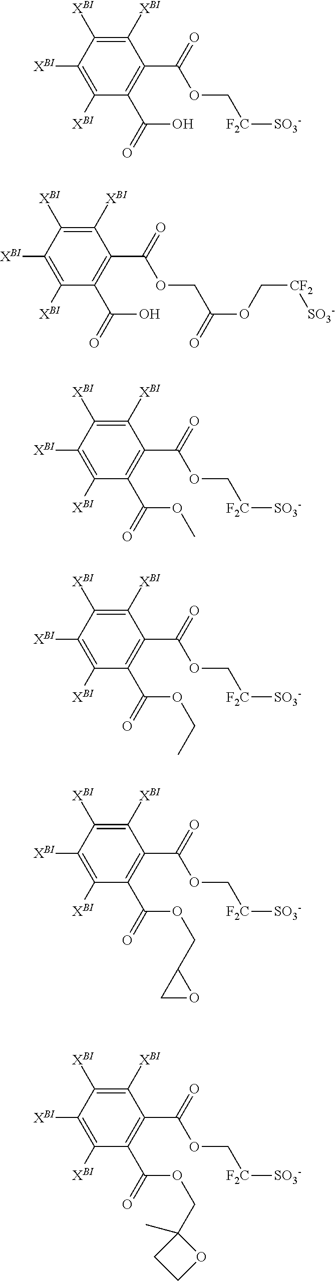

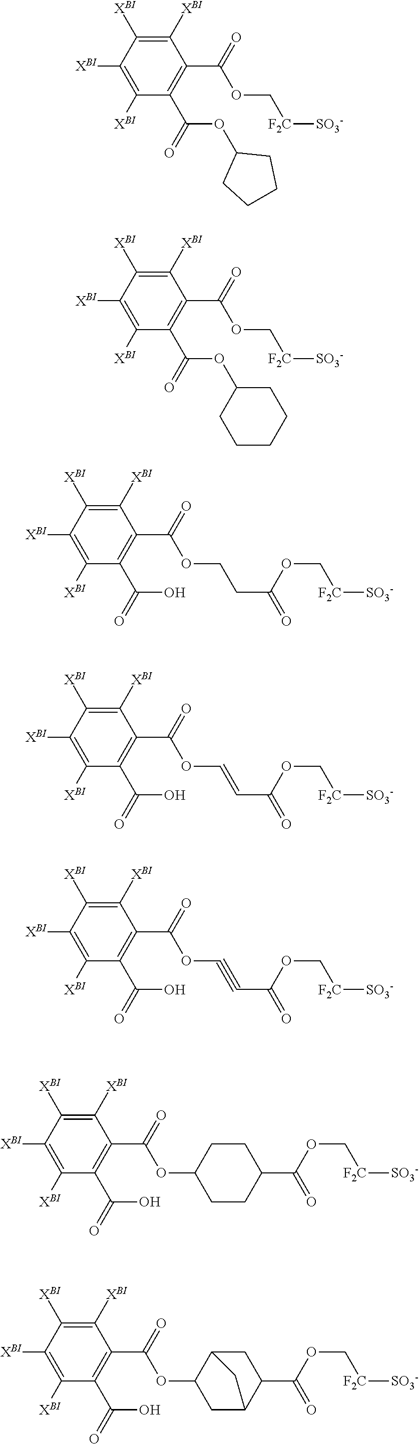

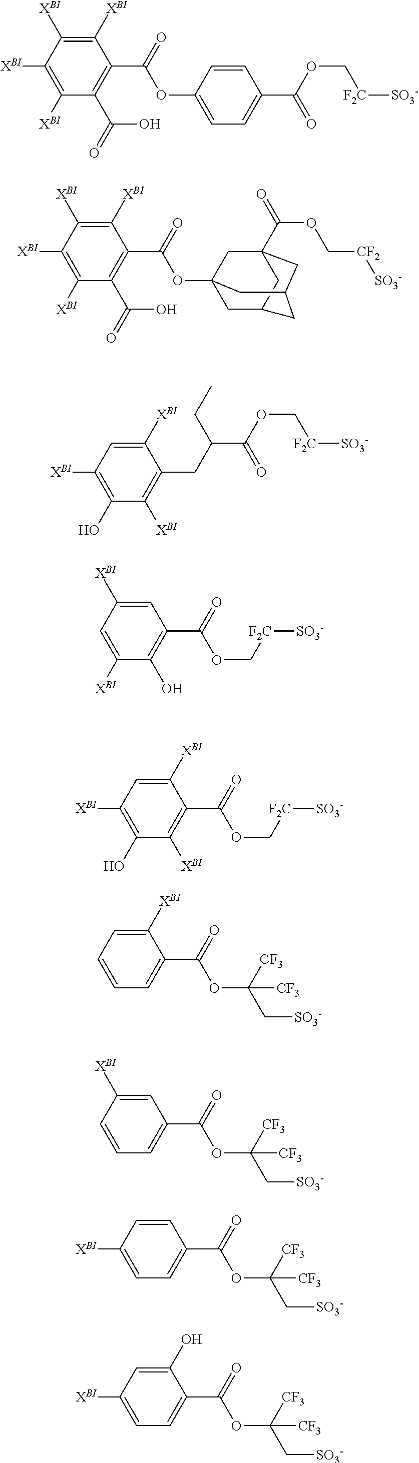

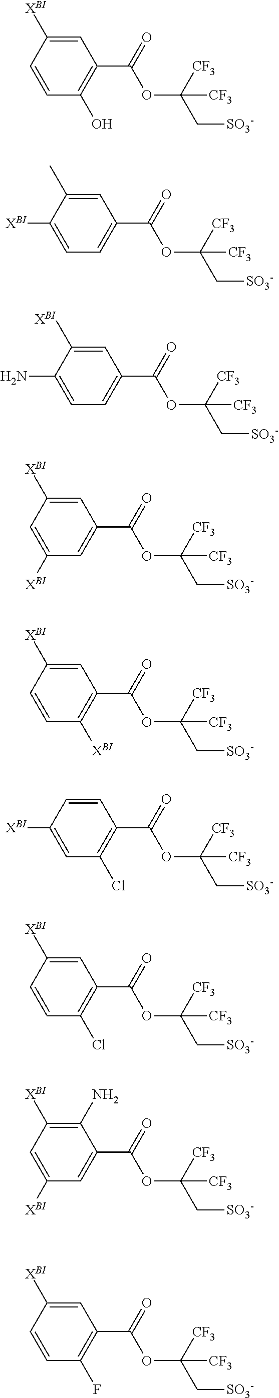

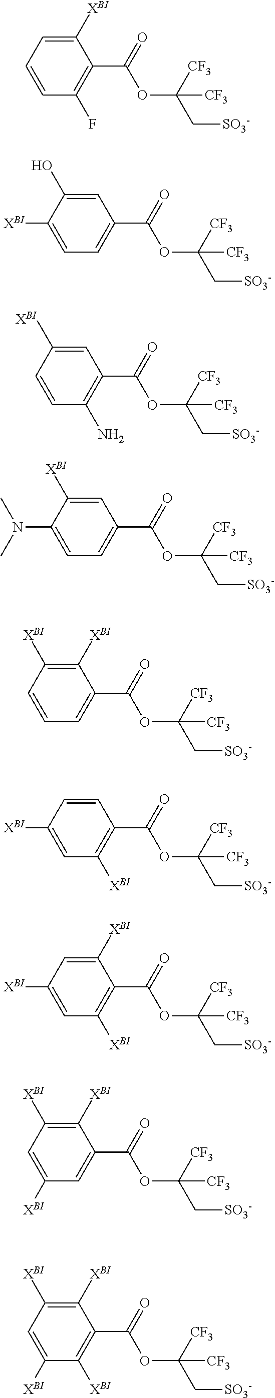

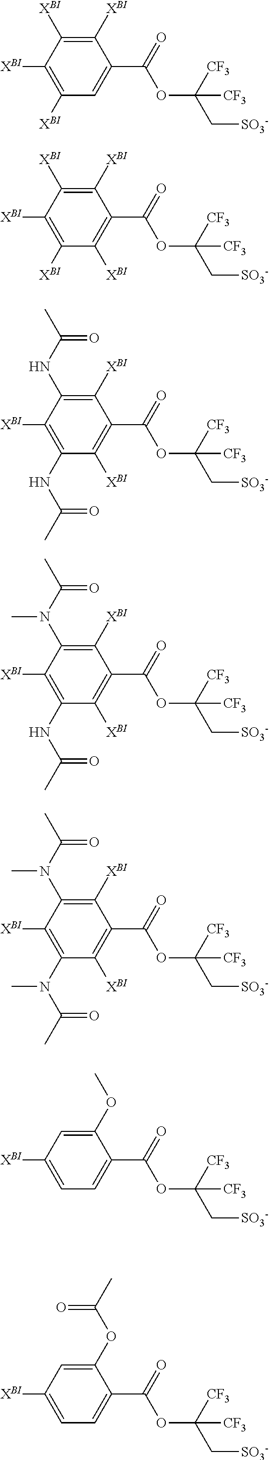

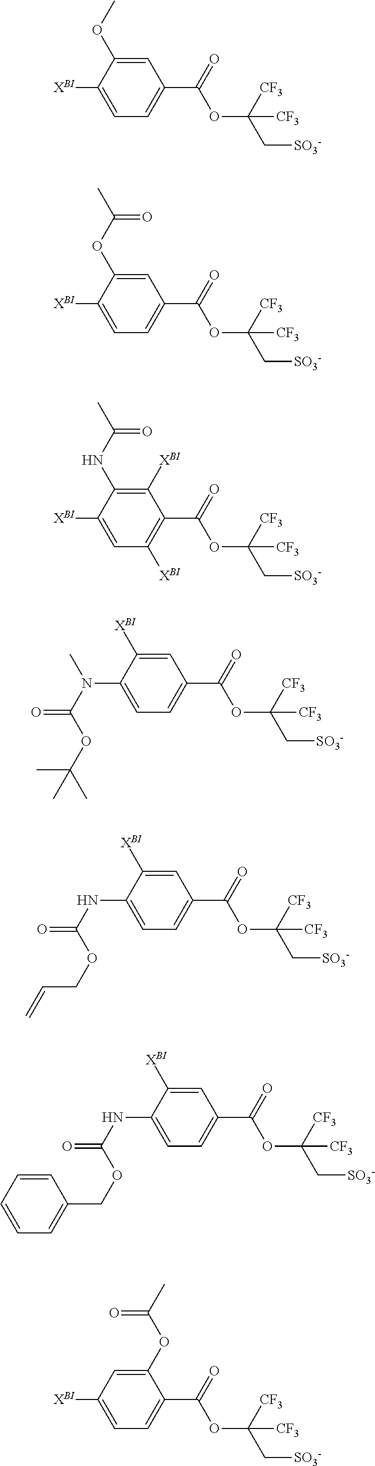

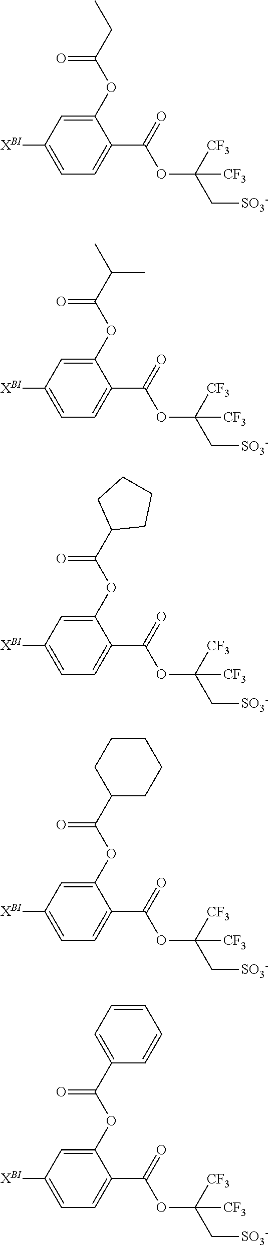

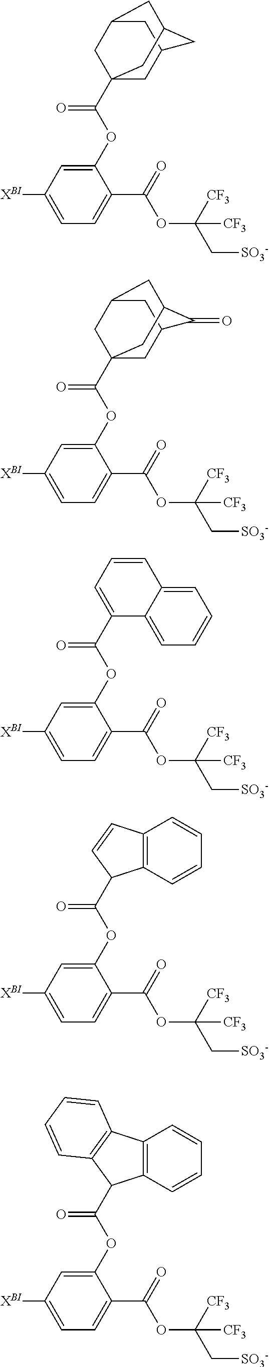

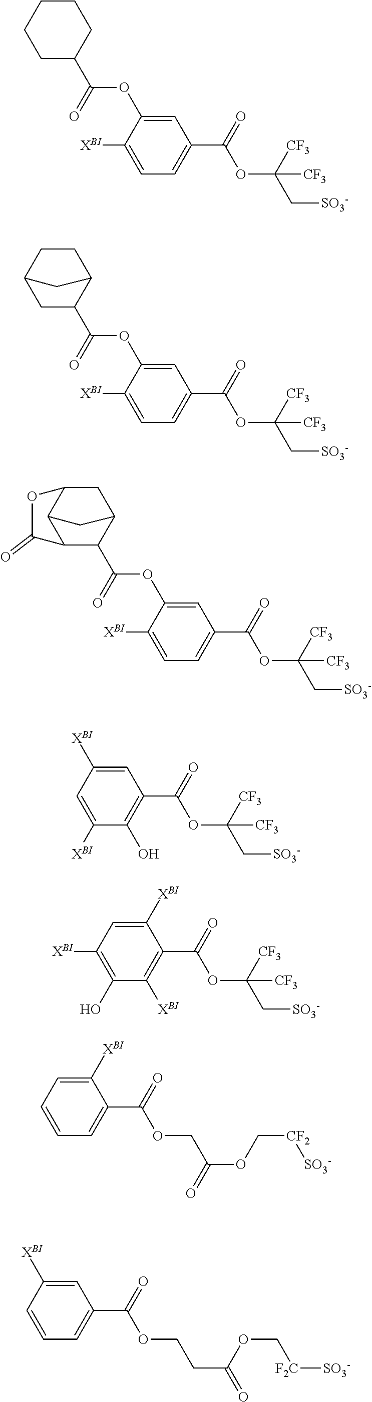

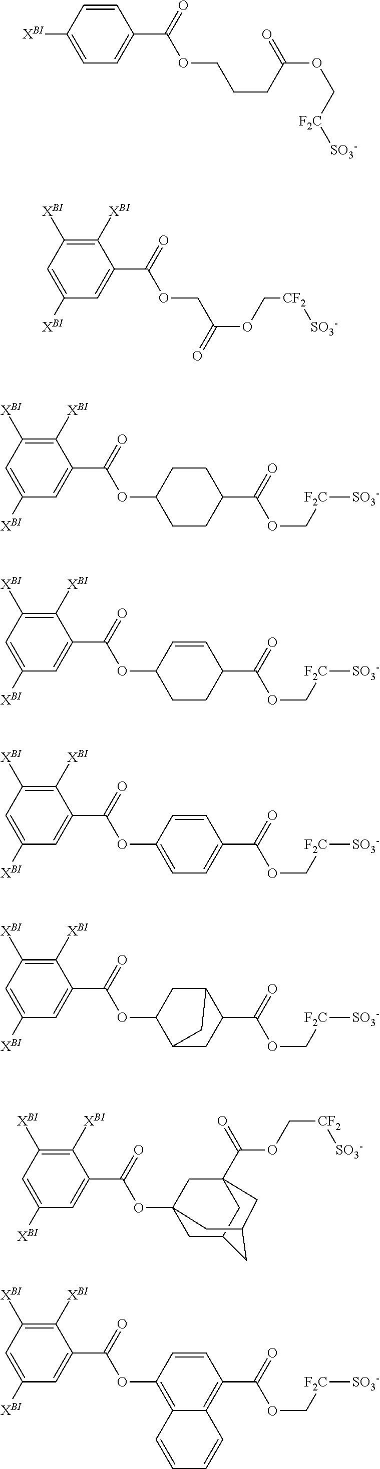

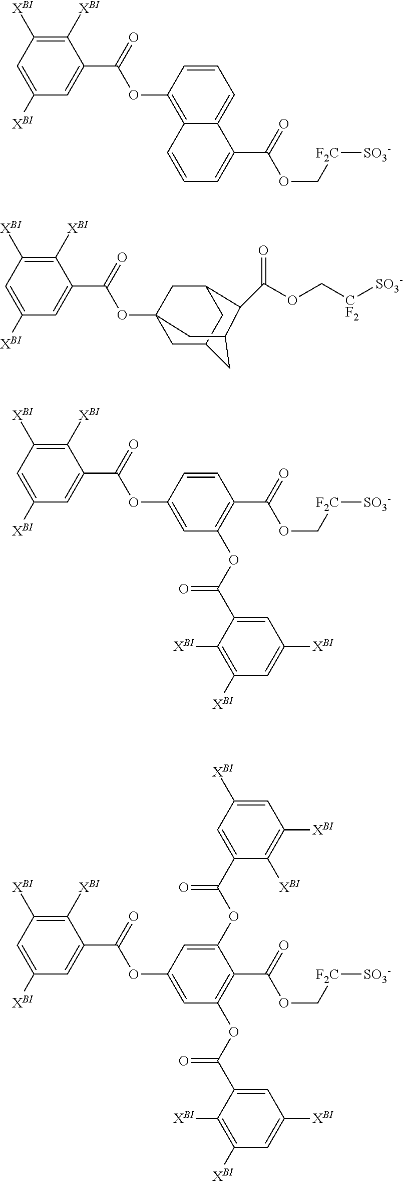

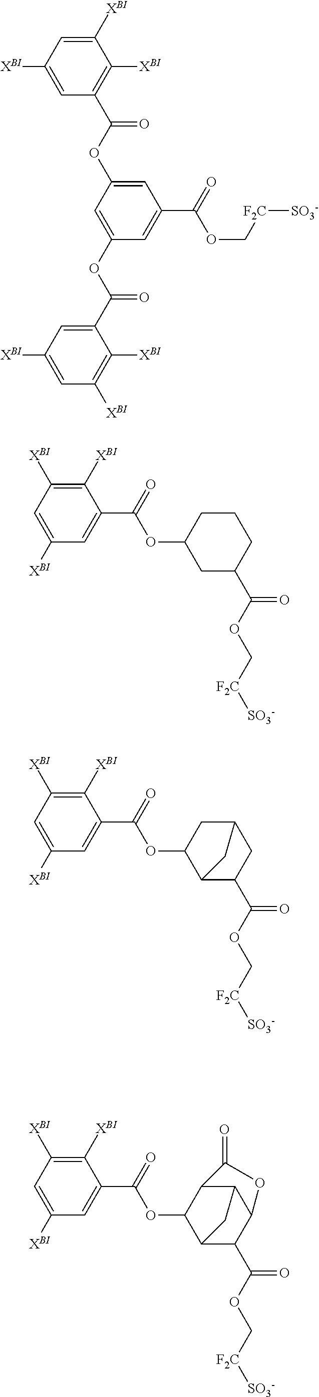

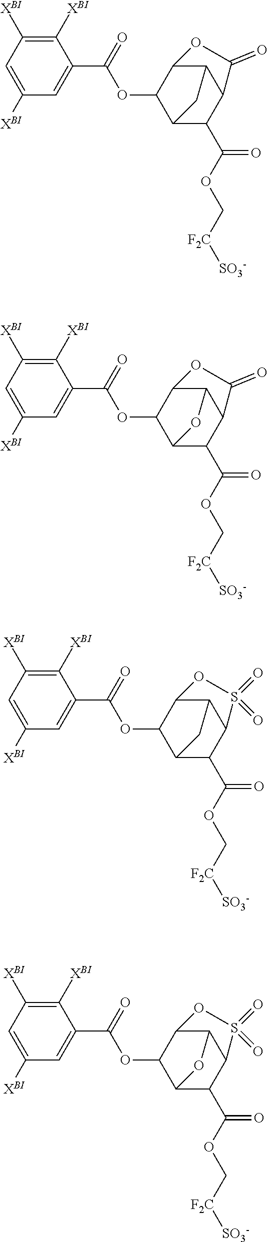

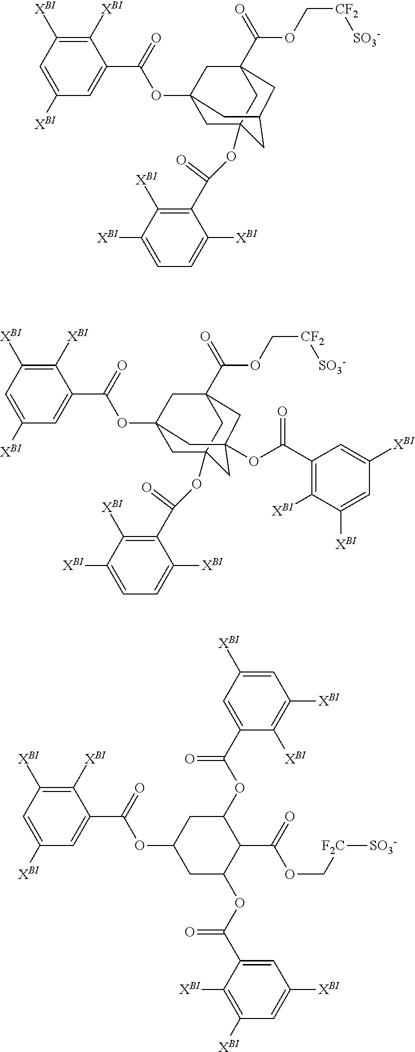

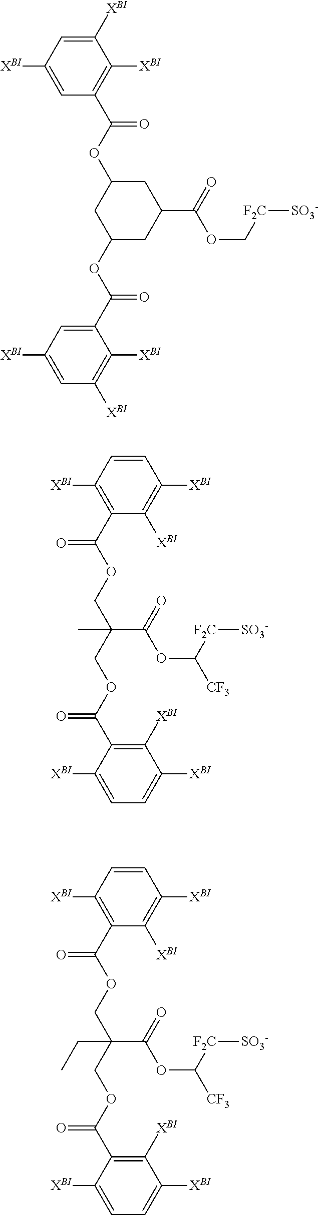

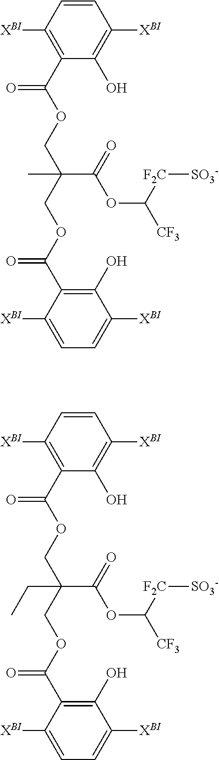

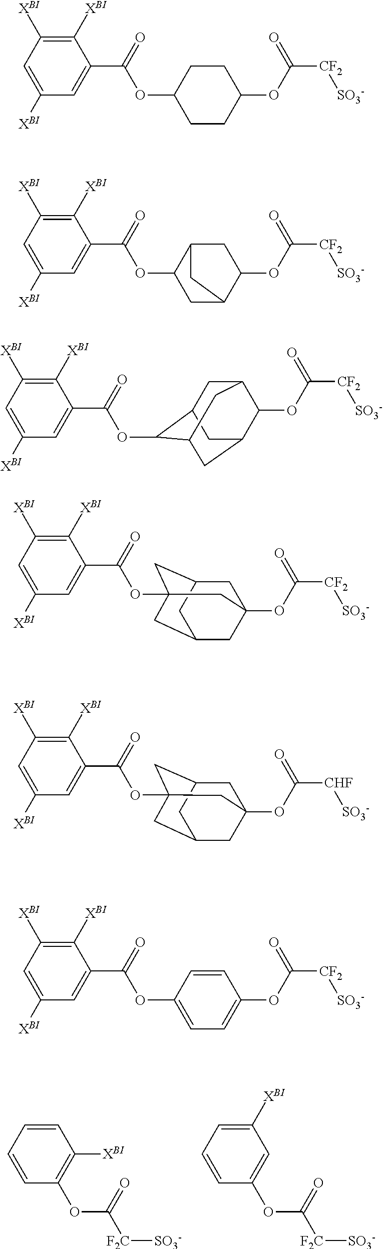

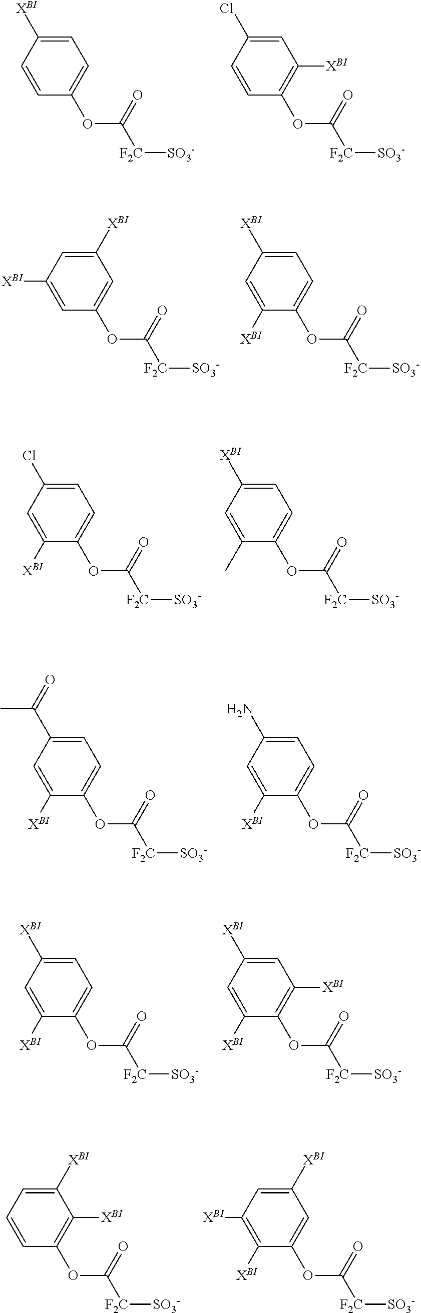

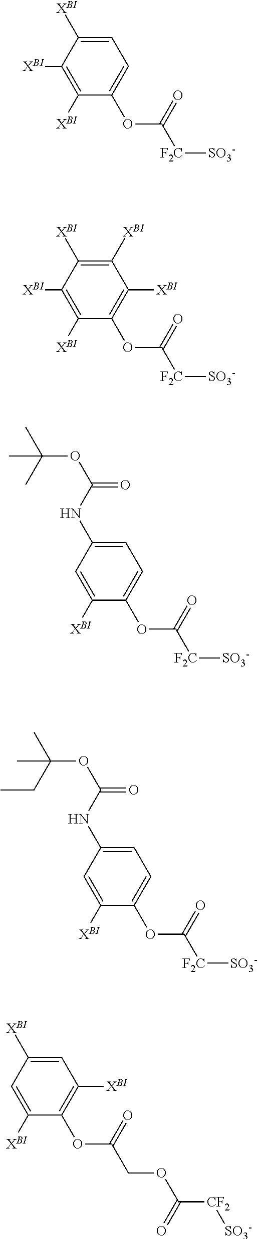

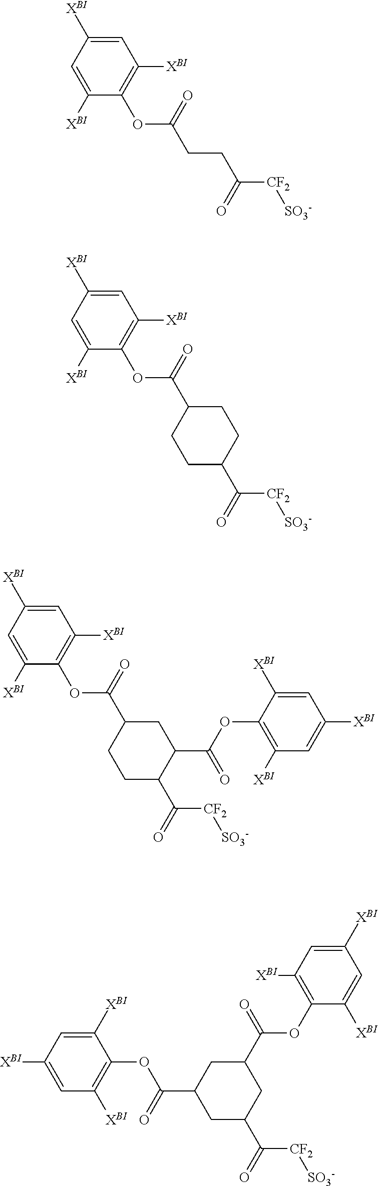

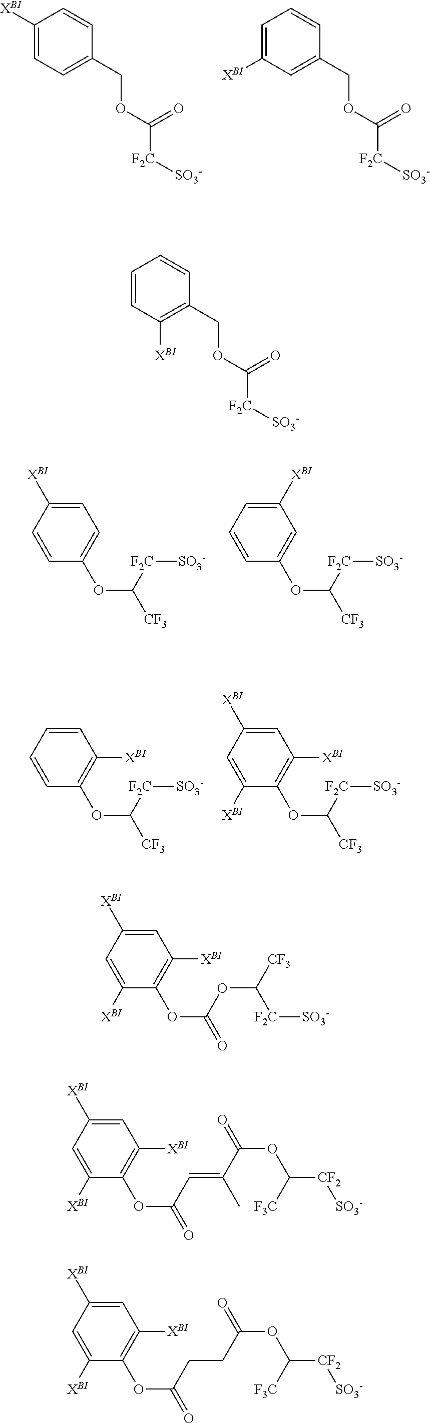

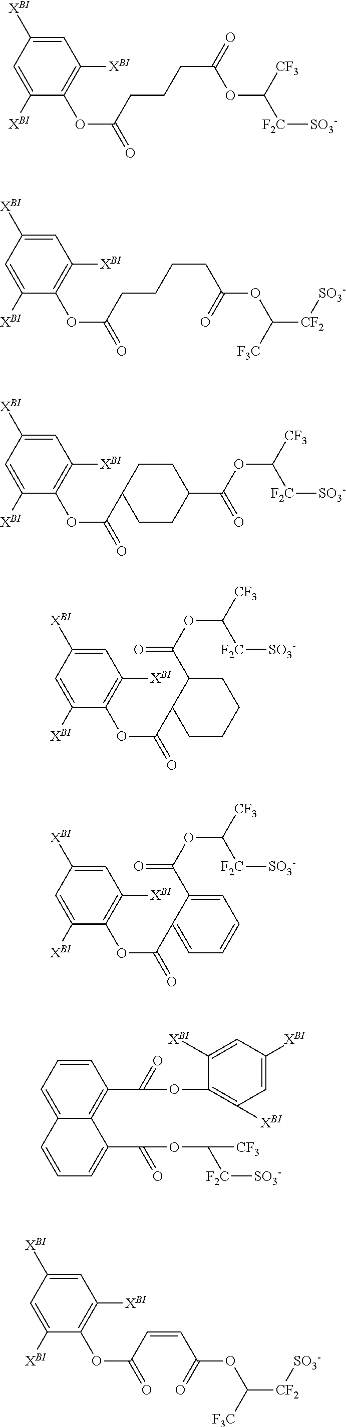

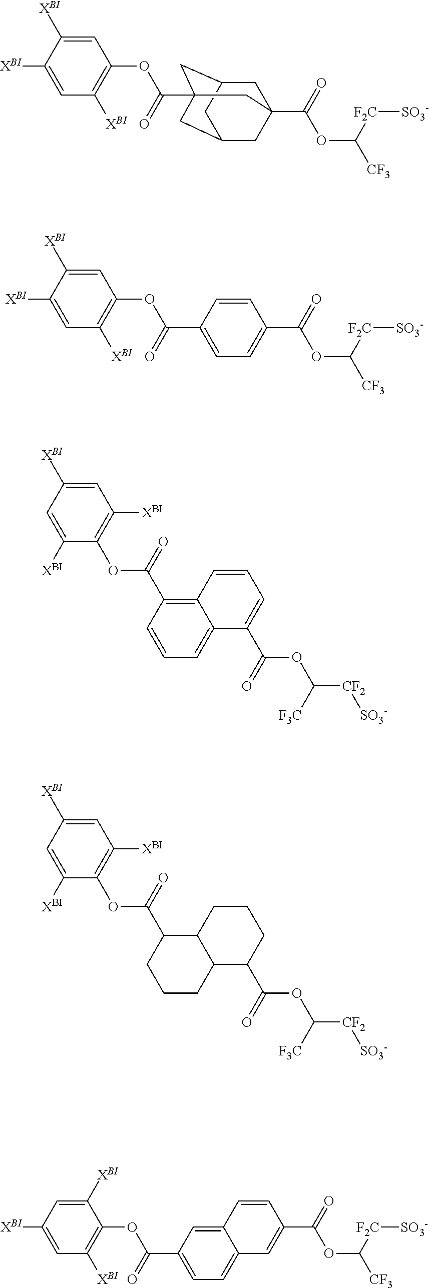

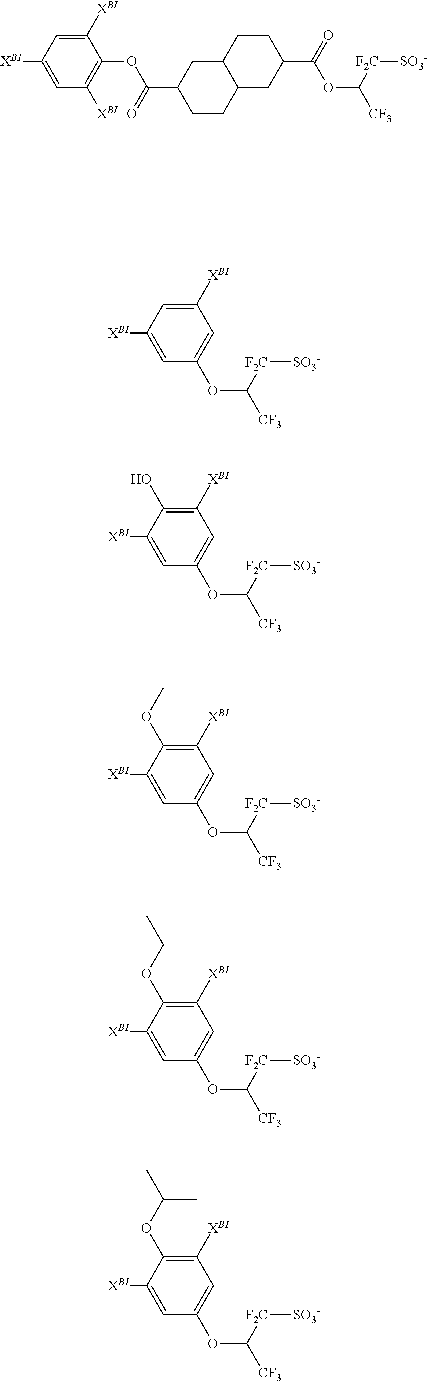

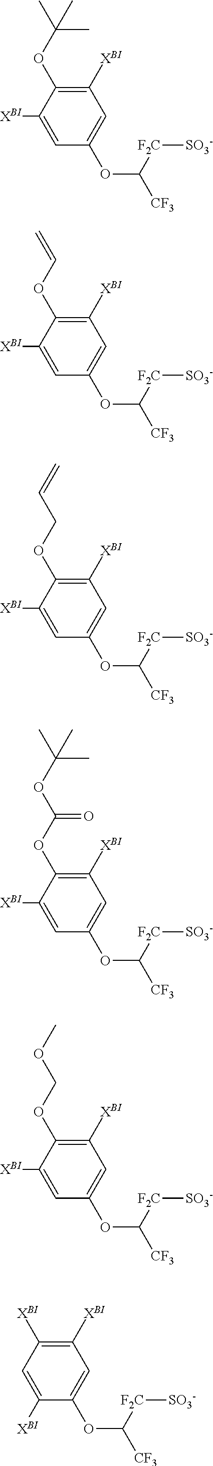

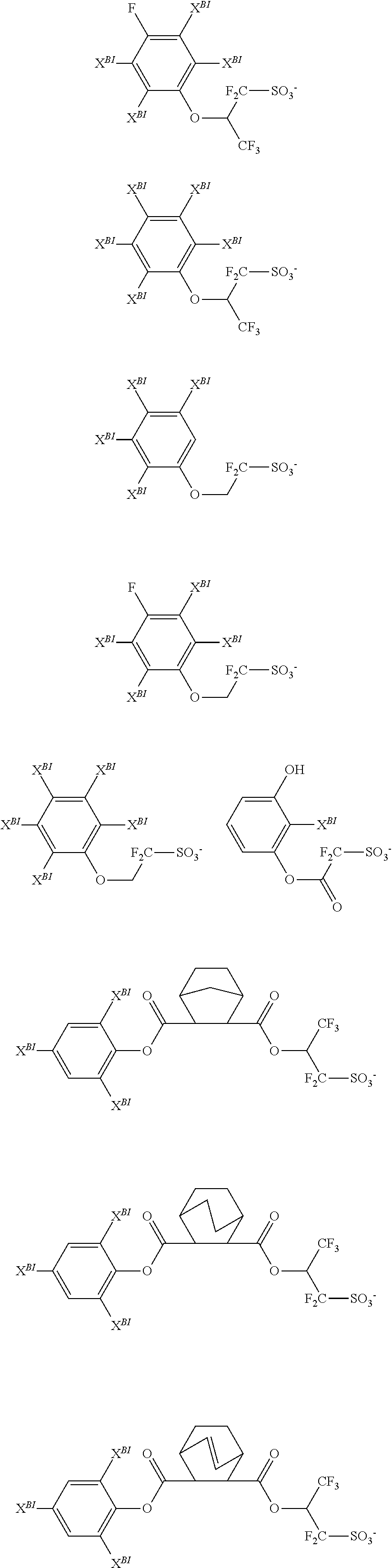

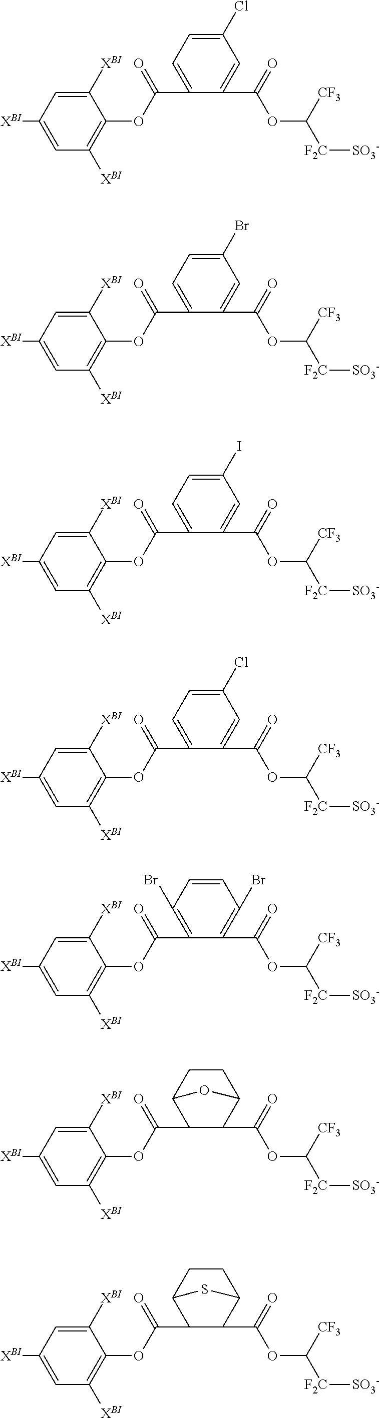

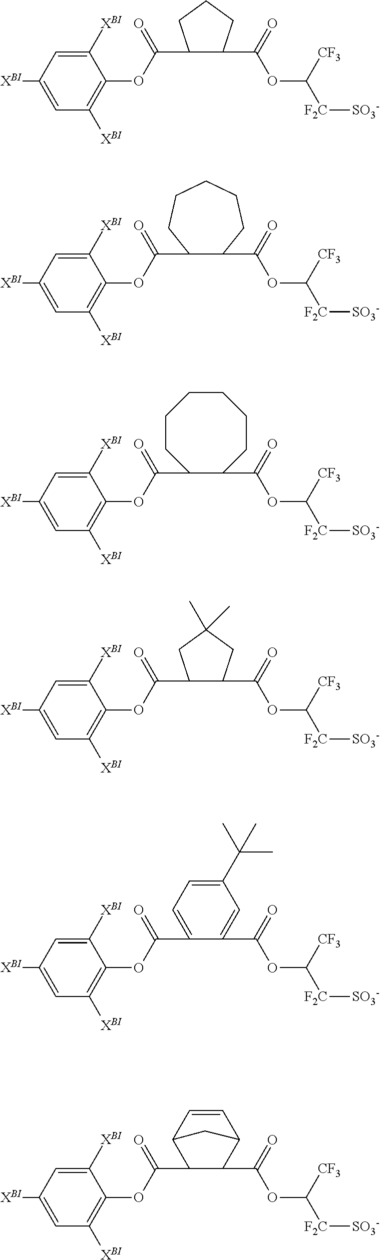

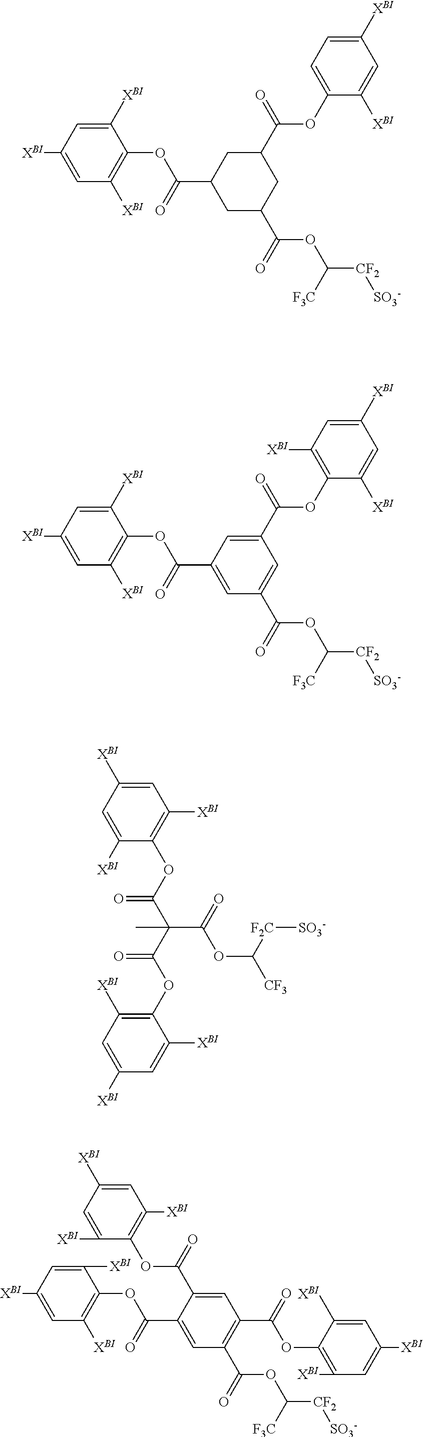

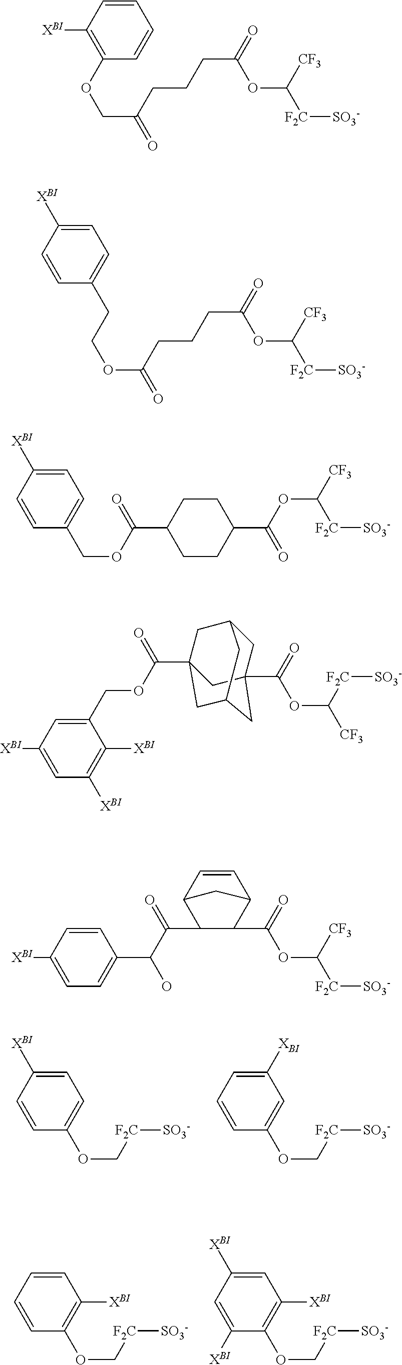

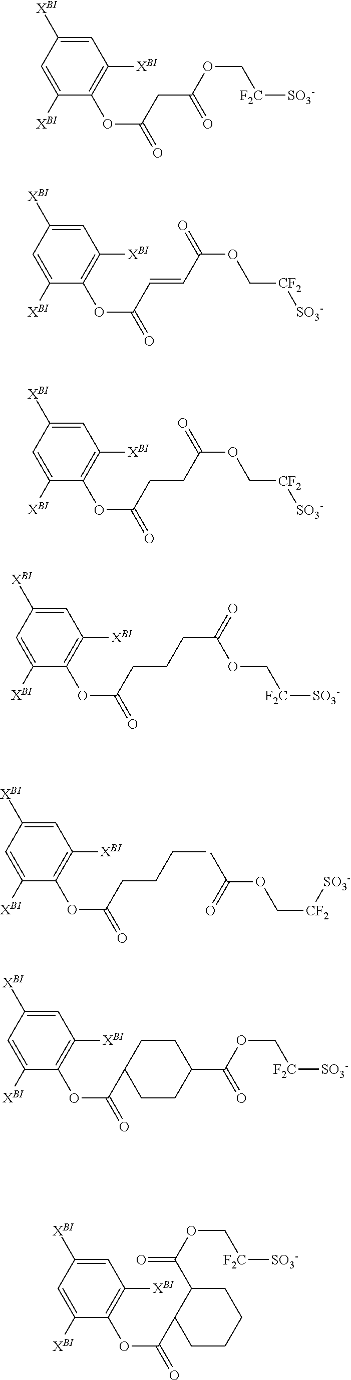

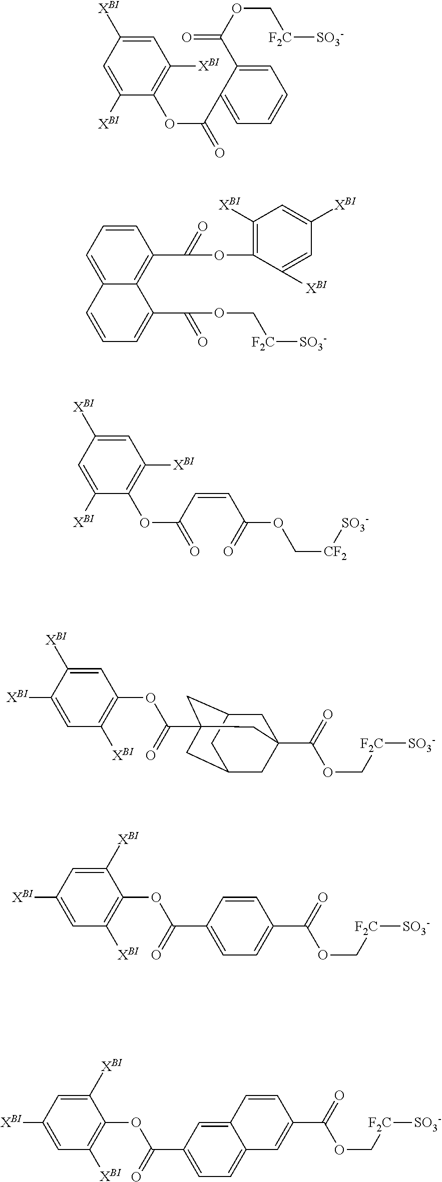

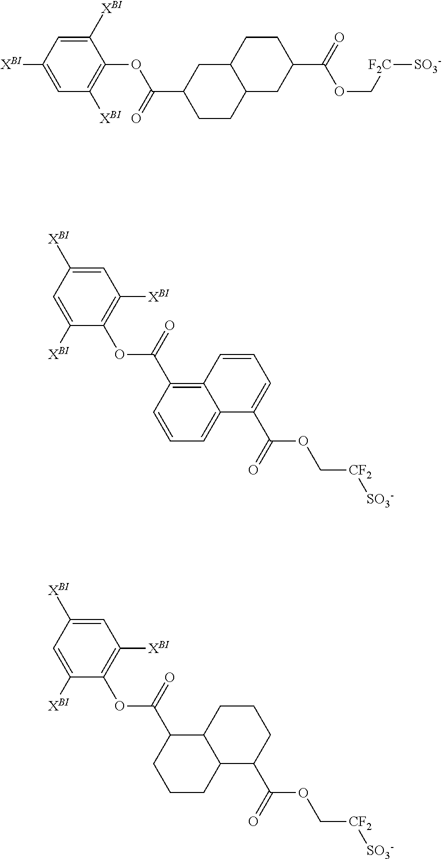

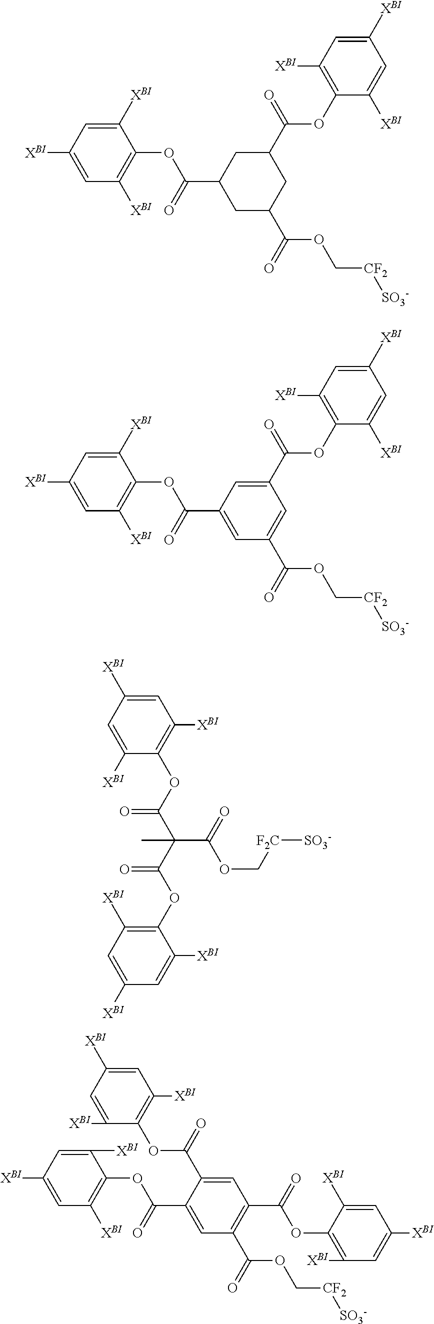

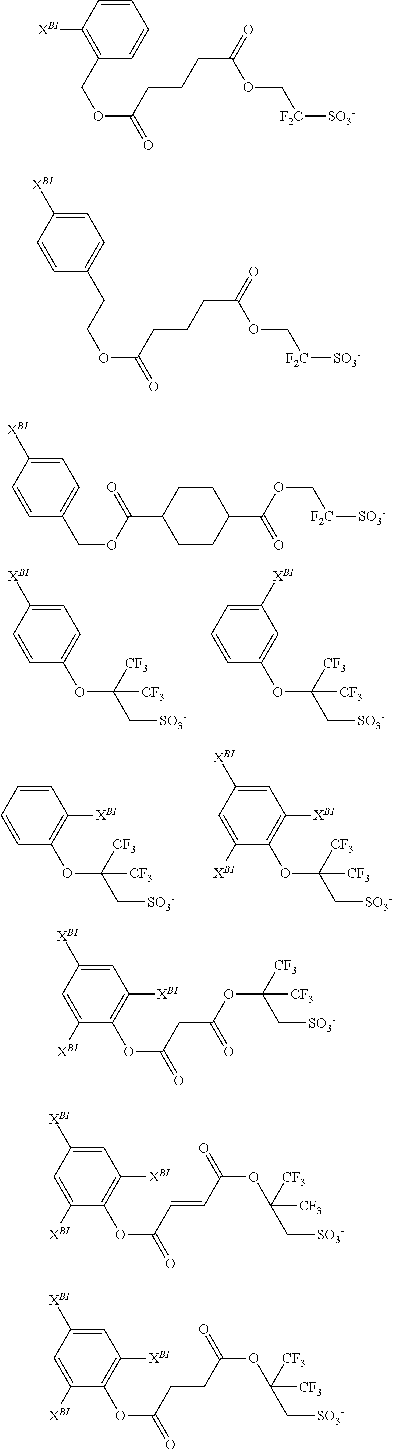

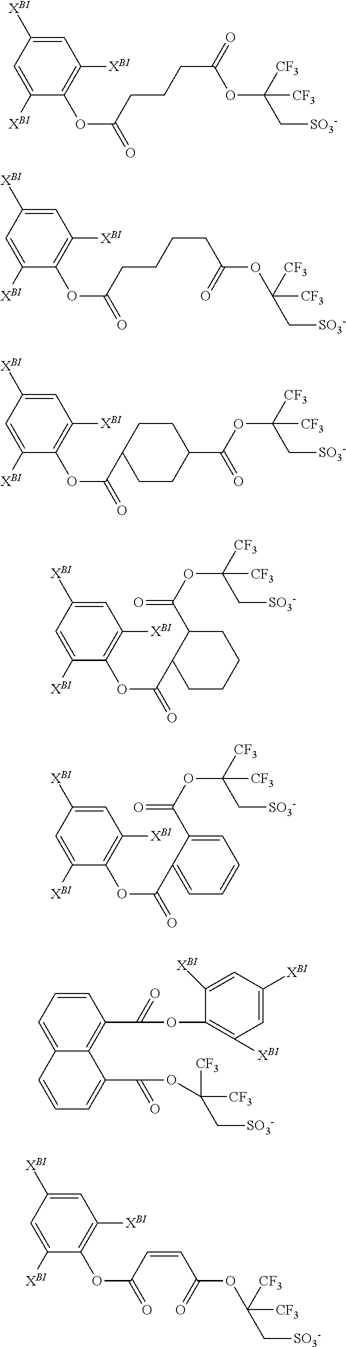

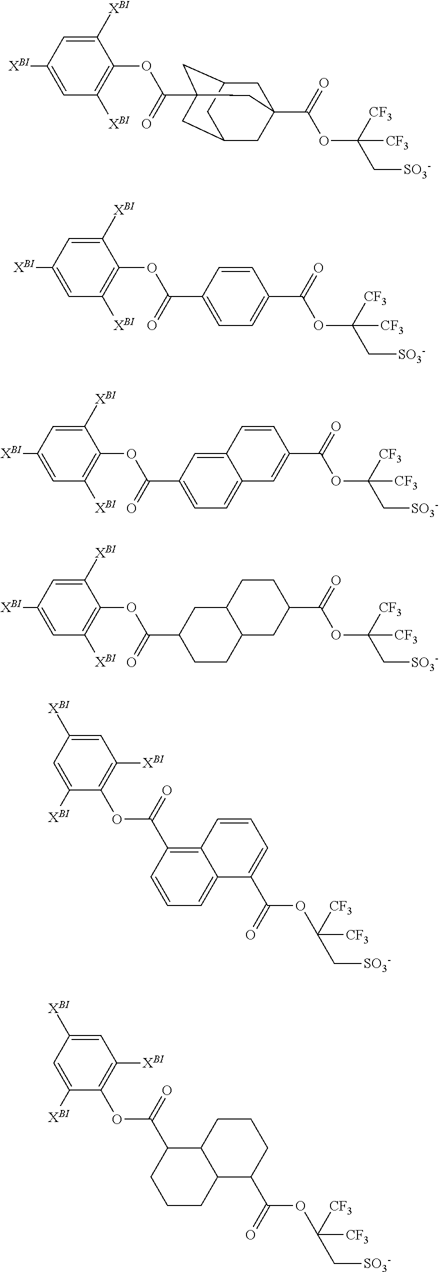

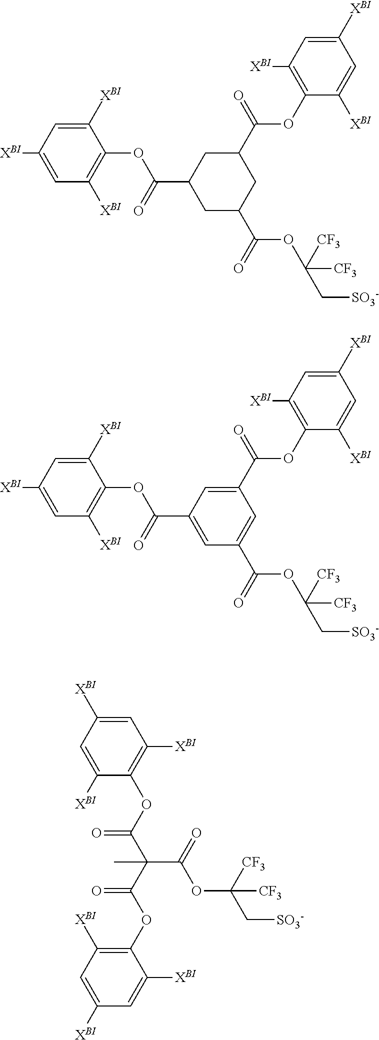

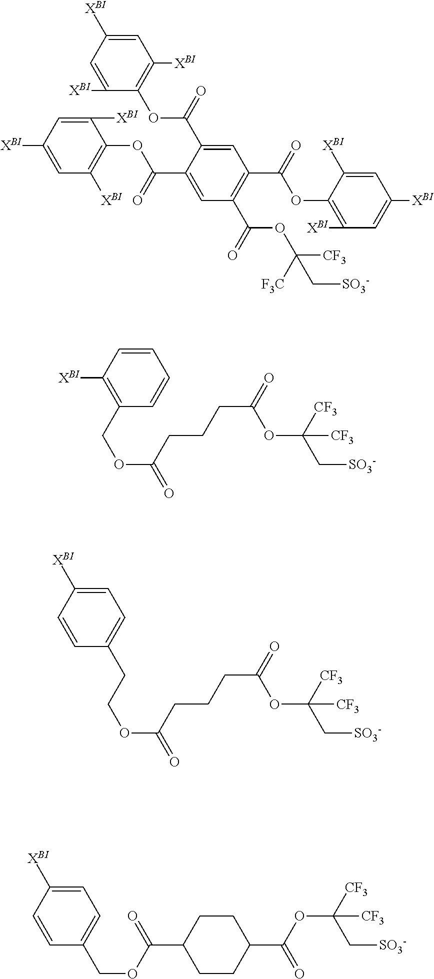

Other useful examples of the non-nucleophilic counter anion include fluorobenzenesulfonic acid anions having an iodized aromatic ring bonded thereto as described in JP 6648726, anions having an acid-catalyzed decomposition mechanism as described in WO 2021/200056 and JP-A 2021-070692, anions having a cyclic ether group as described in JP-A 2018-180525 and JP-A 2021-035935, and anions as described in JP-A 2018-092159.

Further useful examples of the non-nucleophilic counter anion include fluorine-free bulky benzenesulfonic acid anions as described in JP-A 2006-276759, JP-A 2015-117200, JP-A 2016-065016, and JP-A 2019-202974, fluorine-free benzenesulfonic acid or alkylsulfonic acid anions having an iodized aromatic group bonded thereto as described in JP 6645464.

Also useful are bissulfonic acid anions as described in JP-A 2015-206932, sulfonamide or sulfonimide anions having sulfonic acid side and different side as described in WO 2020/158366, and anions having a sulfonic acid side and a carboxylic acid side as described in JP-A 2015-024989.

Also compounds having formula (3) are preferred as the photoacid generator as component (D).

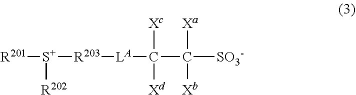

In formula (3), R²⁰¹ and R²⁰² are each independently a C₁-C₃₀ hydrocarbyl group which may contain a heteroatom. R²⁰³ is a C₁-C₃₀ hydrocarbylene group which may contain a heteroatom. Any two of R²⁰¹, R²⁰² and R²⁰³ may bond together to form a ring with the sulfur atom to which they are attached.

The C₁-C₃₀ hydrocarbyl groups represented by R²⁰¹ and R²⁰² may be saturated or unsaturated and straight, branched or cyclic. Examples thereof include C₁-C₃₀ alkyl groups such as methyl, ethyl, n-propyl, isopropyl, n-butyl, sec-butyl, tert-butyl, n-pentyl, tert-pentyl, n-hexyl, n-octyl, 2-ethylhexyl, n-nonyl, and n-decyl; C₃-C₃₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, oxanorbornyl, tricyclo[5.2.1.0^{2,6}]decanyl, and adamantyl; C₆-C₃₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, tert-butylnaphthyl, and anthracenyl; and combinations thereof. In the foregoing hydrocarbyl groups, some or all of the hydrogen atoms may be substituted by a moiety containing a heteroatom such as oxygen, sulfur, nitrogen or halogen, or some constituent —CH₂— may be substituted by a moiety containing a heteroatom such as oxygen, sulfur or nitrogen, so that the group may contain a hydroxy, cyano, fluorine, chlorine, bromine, iodine, carbonyl, ether bond, ester bond, sulfonic ester bond, carbonate bond, lactone ring, sultone ring, carboxylic anhydride (—C(═O)—O—C(═O)—) or haloalkyl moiety.

The C₁-C₃₀ hydrocarbylene group represented by R²⁰³ may be saturated or unsaturated and straight, branched or cyclic. Examples thereof include C₁-C₃₀ alkanediyl groups such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, and heptadecane-1,17-diyl; C₃-C₃₀ cyclic saturated hydrocarbylene groups such as cyclopentanediyl, cyclohexanediyl, norbornanediyl and adamantanediyl; C₆-C₃₀ arylene groups such as phenylene, methylphenylene, ethylphenylene, n-propylphenylene, isopropylphenylene, n-butylphenylene, isobutylphenylene, sec-butylphenylene, tert-butylphenylene, naphthylene, methylnaphthylene, ethylnaphthylene, n-propylnaphthylene, isopropylnaphthylene, n-butylnaphthylene, isobutylnaphthylene, sec-butylnaphthylene, and tert-butylnaphthylene; and combinations thereof. In these hydrocarbylene groups, some or all of the hydrogen atoms may be substituted by a moiety containing a heteroatom such as oxygen, sulfur, nitrogen or halogen, or some constituent —CH₂— may be substituted by a moiety containing a heteroatom such as oxygen, sulfur or nitrogen, so that the group may contain a hydroxy, cyano, fluorine, chlorine, bromine, iodine, carbonyl, ether bond, ester bond, sulfonic ester bond, carbonate bond, lactone ring, sultone ring, carboxylic anhydride (—C(═O)—O—C(═O)—) or haloalkyl moiety. Of the heteroatoms, oxygen is preferred.

In formula (3), L^{A} is a single bond, ether bond or a C₁-C₂₀ hydrocarbylene group which may contain a heteroatom. The hydrocarbylene group may be saturated or unsaturated and straight, branched or cyclic. Examples thereof are as exemplified above for the hydrocarbylene group R²⁰³.

In formula (3), X^{a}, X^{b}, X^{c} and X^{d} are each independently hydrogen, fluorine or trifluoromethyl, with the proviso that at least one of X^{a}, X^{b}, X^{c} and X^{d} is fluorine or trifluoromethyl.

Of the photoacid generators having formula (3), those having formula (3′) are preferred.

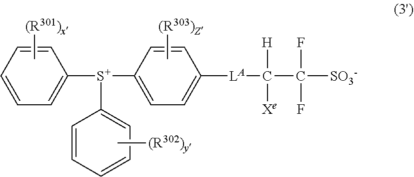

In formula (3′), L^{A} is as defined above. X^{e} is hydrogen or trifluoromethyl, preferably trifluoromethyl. R³⁰¹, R³⁰² and R³⁰³ are each independently hydrogen or a C₁-C₂₀ hydrocarbyl group which may contain a heteroatom. The hydrocarbyl group may be saturated or unsaturated and straight, branched or cyclic. Examples thereof are as exemplified above for the hydrocarbyl group R^{fa1} in formula (2A′). The subscripts x′ and y′ are each independently an integer of 0 to 5, and z′ is an integer of 0 to 4.

Examples of the photoacid generator having formula (3) are as exemplified for the photoacid generator having formula (2) in JP-A 2017-026980.

Of the foregoing photoacid generators, those having an anion of formula (2A′) or (2D) are especially preferred because of reduced acid diffusion and high solubility in the resist solvent. Also those having formula (3′) are especially preferred because of extremely reduced acid diffusion.

When the resist composition of the invention contains the photoacid generator (D), it is preferably used in an amount of 0.1 to 40 parts by weight, and more preferably 0.5 to 20 parts by weight per 80 parts by weight of the base polymer (A). An amount of the photoacid generator (D) in the range ensures good resolution and eliminates the risk of leaving foreign matter after development or during separation of resist film. The photoacid generator (D) may be used alone or in admixture of two or more. When the base polymer contains the repeat units (d1) to (d4) and/or the resist composition contains the photoacid generator (D), the resist composition of the invention functions as a chemically amplified resist composition.

### (E) Quencher

The chemically amplified resist composition of the invention preferably comprises a quencher, that is, acid diffusion controlling agent. As used herein, the quencher refers to a compound capable of trapping the acid generated by the photoacid generator in the chemically amplified resist composition to prevent the acid from diffusing to the unexposed region, for thereby forming the desired pattern. Onium salts having formula (4) or (5) are useful as the quencher.

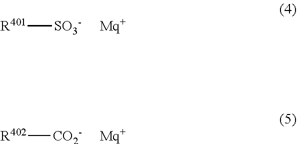

In formula (4), R⁴⁰¹ is hydrogen or a C₁-C₄₀ hydrocarbyl group which may contain a heteroatom, exclusive of the hydrocarbyl group in which the hydrogen atom bonded to the carbon atom at α-position of the sulfo group is substituted by fluorine or fluoroalkyl.

The hydrocarbyl group represented by R⁴⁰¹ may be saturated or unsaturated and straight, branched or cyclic. Examples thereof include C₁-C₄₀ alkyl groups such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl, and n-decyl; C₃-C₄₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, oxanorbornyl, tricyclo[5.2.1.0^{2,6}]decanyl, and adamantyl; C₆-C₄₀ aryl groups such as phenyl, naphthyl, and anthracenyl; and combinations thereof. In the foregoing hydrocarbyl groups, some or all of the hydrogen atoms may be substituted by a moiety containing a heteroatom such as oxygen, sulfur, nitrogen or halogen, or some constituent —CH₂— may be substituted by a moiety containing a heteroatom such as oxygen, sulfur or nitrogen, so that the group may contain a hydroxy, cyano, fluorine, chlorine, bromine, iodine, carbonyl, ether bond, ester bond, sulfonic ester bond, carbonate bond, lactone ring, sultone ring, carboxylic anhydride (—C(═O)—O—C(═O)—) or haloalkyl moiety.

In formula (5), R⁴⁰² is hydrogen or a C₁-C₄₀ hydrocarbyl group which may contain a heteroatom. Examples of the hydrocarbyl group which may contain a heteroatom include the substituent group exemplified above for R⁴⁰¹ and fluorinated saturated hydrocarbyl groups such as trifluoromethyl and trifluoroethyl, and fluorinated aryl groups such as pentafluorophenyl and 4-trifluoromethylphenyl.

Examples of the anion in the onium salt having formula (4) are shown below, but not limited thereto.

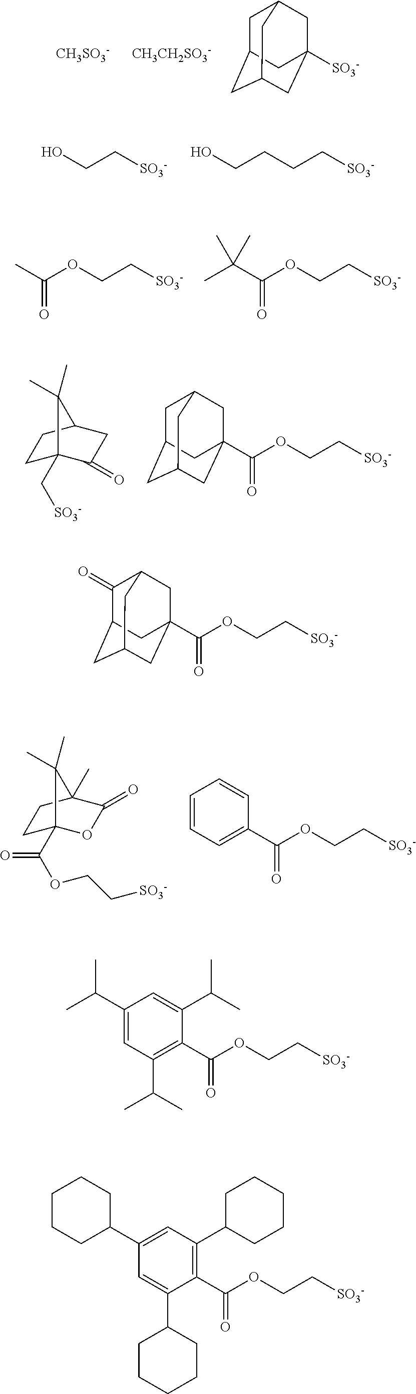

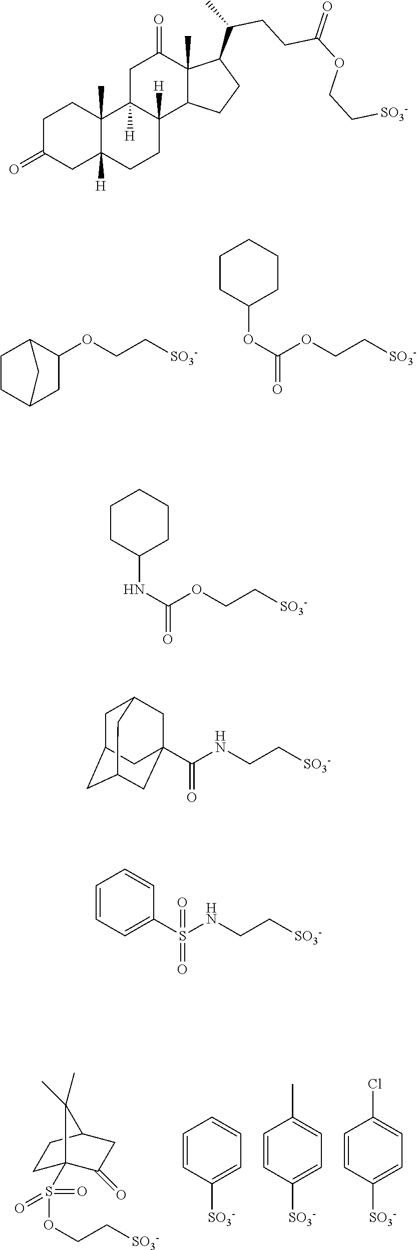

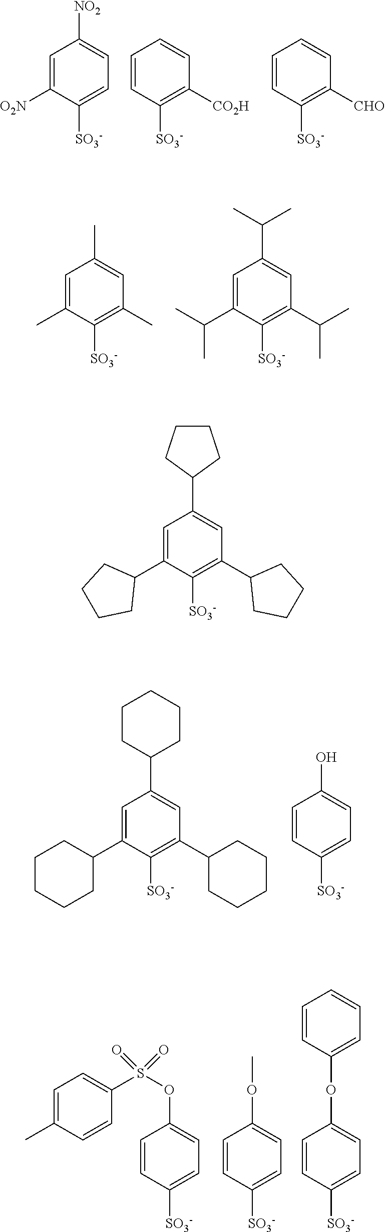

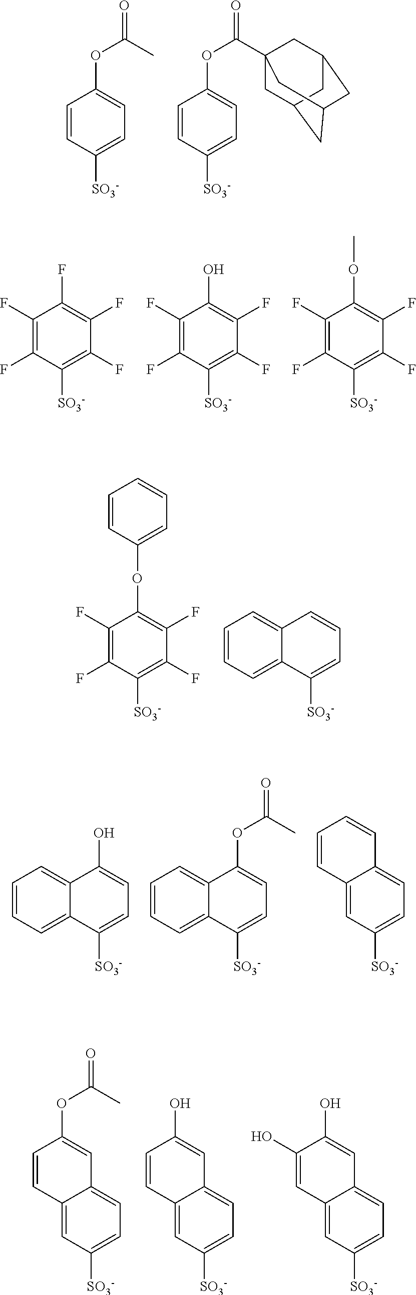

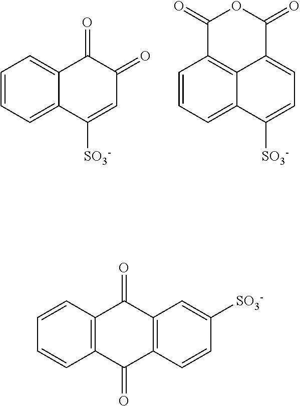

Examples of the anion in the onium salt having formula (5) are shown below, but not limited thereto.

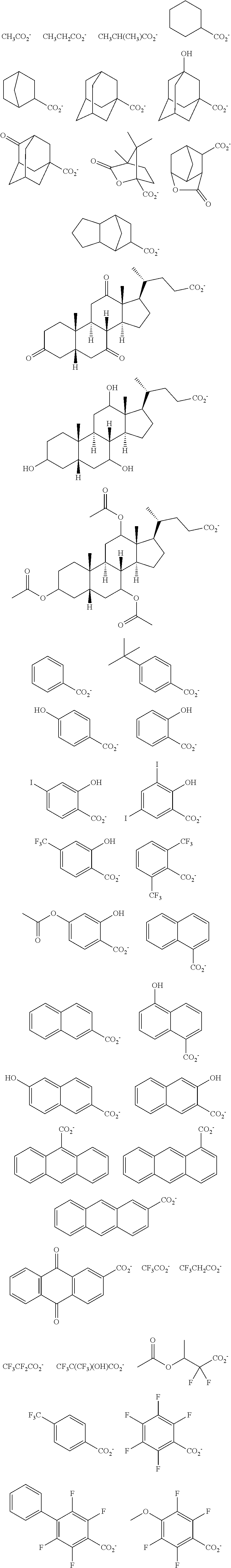

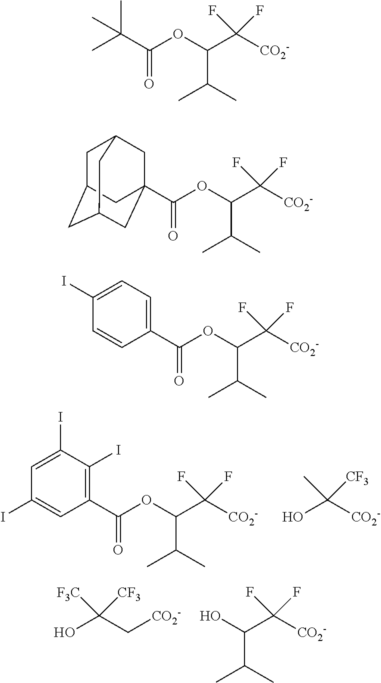

In formulae (3) and (4), Mq⁺ is an onium cation. The onium cation is preferably selected from sulfonium cations having formula (6-1), iodonium cations having formula (6-2), and ammonium cations having formula (6-3).

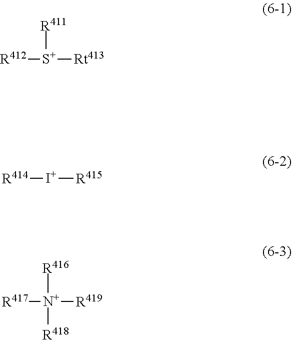

In formulae (6-1) to (6-3), R⁴¹¹ to R⁴¹⁹ are each independently a C₁-C₄₀ hydrocarbyl group which may contain a heteroatom. A pair of R⁴¹¹ and R⁴¹² may bond together to form a ring with the sulfur atom to which they are attached, and a pair of R⁴¹⁶ and R⁴¹⁷ may bond together to form a ring with the nitrogen atom to which they are attached. Examples of the hydrocarbyl group are as exemplified above for R⁴⁰¹ in formula (3).

Examples of the sulfonium cation having formula (6-1) are shown below, but not limited thereto.

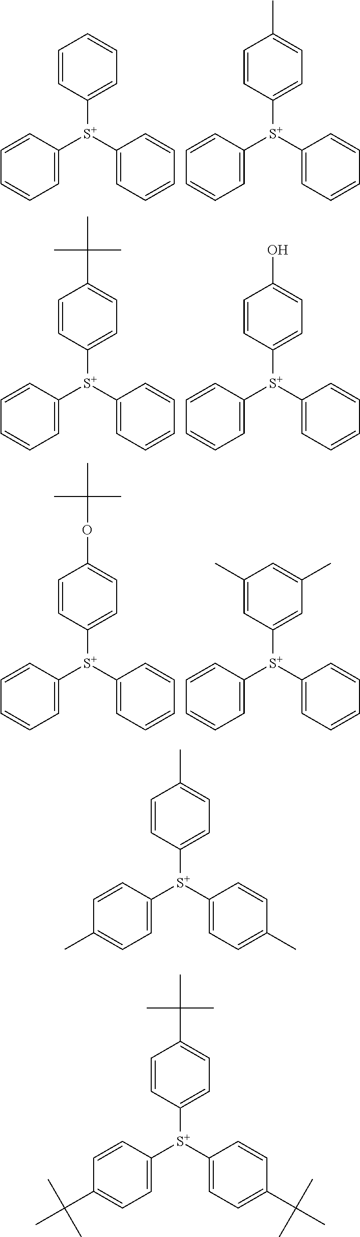

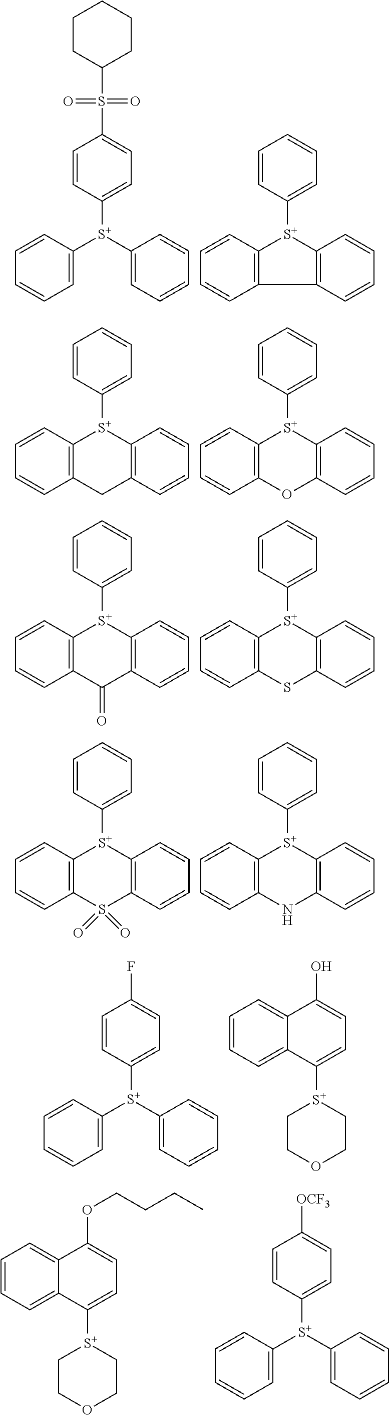

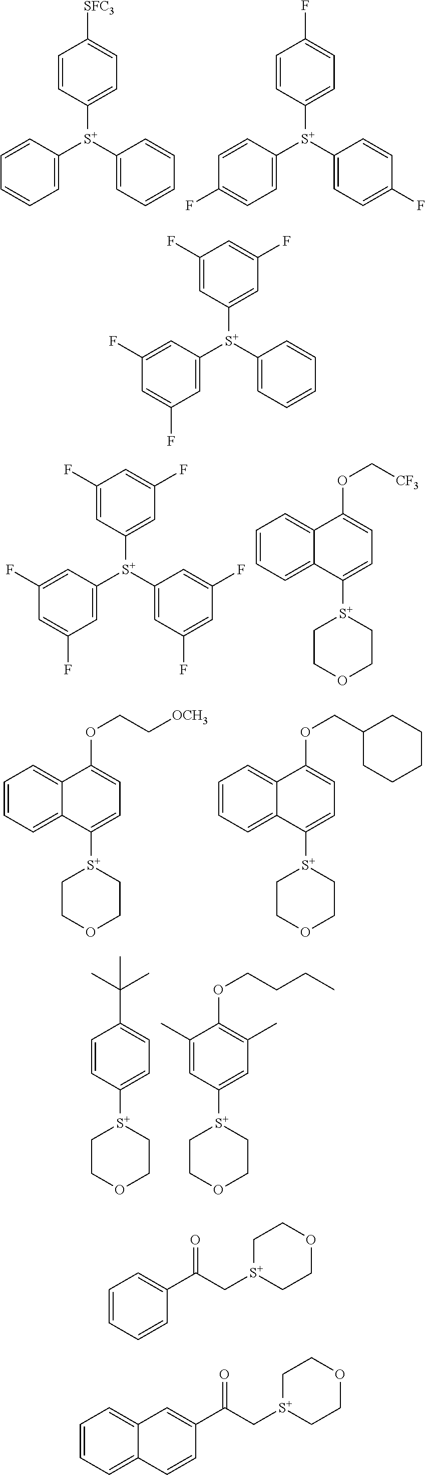

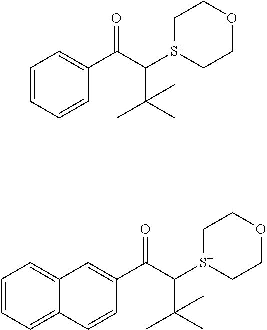 [SMILES: CC(C)(C)C(C(=O)c1ccccc1)[S+]1CCOCC1 ; CC(C)(C)C(C(=O)c1ccc2ccccc2c1)[S+]1CCOCC1]

Examples of the iodonium cation having formula (6-2) are shown below, but not limited thereto.

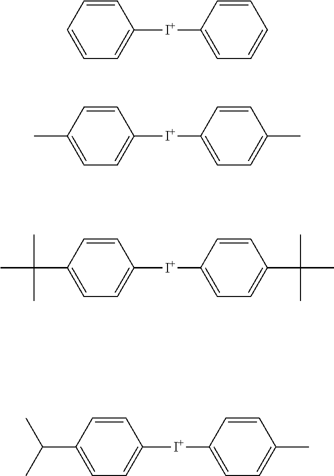

Examples of the ammonium cation having formula (6-3) are shown below, but not limited thereto.

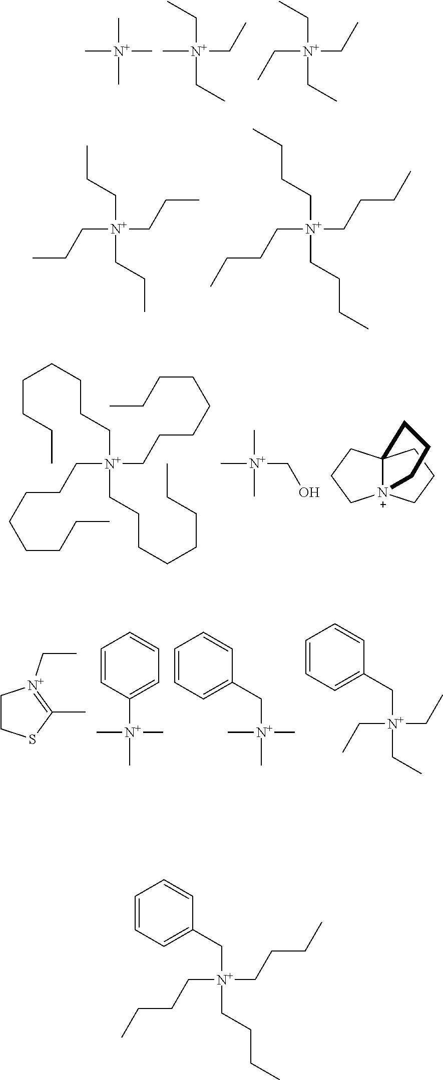

Examples of the onium salt having formula (4) or (5) include arbitrary combinations of anions with cations, both as exemplified above. These onium salts may be readily prepared by ion exchange reaction using any well-known organic chemistry technique. For the ion exchange reaction, reference may be made to JP-A 2007-145797, for example.

The onium salt having formula (4) or (5) functions as a quencher in the chemically amplified resist composition of the invention. This is because the counter anion of the onium salt is a conjugated base of a weak acid. As used herein, the term “weak acid” indicates an acidity insufficient to deprotect an acid labile group from an acid labile group containing unit in the base polymer. The onium salt having formula (4) or (5) functions as a quencher when used in combination with an onium salt type photoacid generator having a conjugated base of a strong acid (typically a sulfonic acid which is fluorinated at α-position) as the counter anion. In a system using a mixture of an onium salt capable of generating a strong acid (e.g., α-position fluorinated sulfonic acid) and an onium salt capable of generating a weak acid (e.g., non-fluorinated sulfonic acid or carboxylic acid), if the strong acid generated from the photoacid generator upon exposure to high-energy radiation collides with the unreacted onium salt having a weak acid anion, then a salt exchange occurs whereby the weak acid is released and an onium salt having a strong acid anion is formed. In this course, the strong acid is exchanged into the weak acid having a low catalysis, incurring apparent deactivation of the acid for enabling to control acid diffusion.

If a photoacid generator capable of generating a strong acid is an onium salt, an exchange from the strong acid generated upon exposure to high-energy radiation to a weak acid as above can take place, but it rarely happens that the weak acid generated upon exposure to high-energy radiation collides with the unreacted onium salt capable of generating a strong acid to induce a salt exchange. This is because of a likelihood of an onium cation forming an ion pair with a stronger acid anion.

When the resist composition of the invention contains the onium salt having formula (4) or (5) as the quencher (E), the amount of the onium salt used is preferably 0.1 to 10 parts by weight, more preferably 0.1 to 5 parts by weight per 80 parts by weight of the base polymer (A).

Also nitrogen-containing compounds may be used as the quencher as component (E). Suitable nitrogen-containing compounds include primary, secondary and tertiary amine compounds, specifically amine compounds having a hydroxy group, ether bond, ester bond, lactone ring, cyano group or sulfonic ester bond, as described in JP-A 2008-111103, paragraphs [0146]-[0164](U.S. Pat. No. 7,537,880). Suitable nitrogen-containing compounds also include primary or secondary amine compounds protected with a carbamate group, as described in JP 3790649

A sulfonic acid sulfonium salt having a nitrogen-containing substituent may also be used as the nitrogen-containing compound. This compound functions as a quencher in the unexposed region, but as a so-called photo-degradable base in the exposed region because it loses the quencher function in the exposed region due to neutralization thereof with the acid generated by itself. Using a photo-degradable base, the contrast between exposed and unexposed regions can be further enhanced. With respect to the photo-degradable base, reference may be made to JP-A 2009-109595 and JP-A 2012-046501, for example.

When the resist composition of the invention contains the nitrogen-containing compound as the quencher, the amount of the nitrogen-containing compound used is preferably 0.001 to 12 parts by weight, more preferably 0.01 to 8 parts by weight per 80 parts by weight of the base polymer (A). The nitrogen-containing compound may be used alone or in admixture of two or more.

### (F) Surfactant

The resist composition of the invention may further comprise a surfactant as component (F). It is typically a surfactant (F) which is insoluble or substantially insoluble in water and alkaline developer, or a surfactant which is insoluble or substantially insoluble in water and soluble in alkaline developer. For the surfactant, reference should be made to those compounds described in JP-A 2010-215608 and JP-A 2011-016746.

While many examples of the surfactant which is insoluble or substantially insoluble in water and alkaline developer are described in the patent documents cited herein, preferred examples are fluorochemical surfactants FC-4430 (3M), Olfine® E1004 (Nissin Chemical Co., Ltd.), Surflon® 5-381, KH-20 and KH-30 (AGC Seimi Chemical Co., Ltd.). Partially fluorinated oxetane ring-opened polymers having formula (surf-1) are also useful.

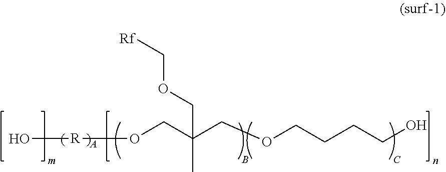

It is provided herein that R, Rf, A, B, C, m, and n are applied to only formula (surf-1), independent of their descriptions other than for the surfactant. R is a di- to tetra-valent C₂-C₅ aliphatic group. Exemplary divalent aliphatic groups include ethylene, 1,4-butylene, 1,2-propylene, 2,2-dimethyl-1,3-propylene and 1,5-pentylene. Exemplary tri- and tetra-valent groups are shown below.

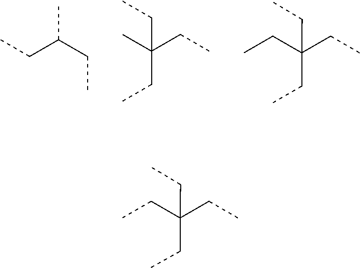

Herein the broken line is a valence bond. These formulae are partial structures derived from glycerol, trimethylol ethane, trimethylol propane, and pentaerythritol, respectively.

Of these, 1,4-butylene and 2,2-dimethyl-1,3-propylene are preferred.

Rf is trifluoromethyl or pentafluoroethyl, and preferably trifluoromethyl. The subscript m is an integer of 0 to 3, n is an integer of 1 to 4, and the sum of m and n, which represents the valence of R, is an integer of 2 to 4. “A” is equal to 1. B is an integer of 2 to 25, preferably an integer of 4 to 20. C is an integer of 0 to 10, preferably 0 or 1. Note that formula (surf-1) does not prescribe the arrangement of respective constituent units while they may be arranged either blockwise or randomly. For the preparation of surfactants in the form of partially fluorinated oxetane ring-opened polymers, reference should be made to U.S. Pat. No. 5,650,483, for example.

The surfactant which is insoluble or substantially insoluble in water and soluble in alkaline developer is useful when ArF immersion lithography is applied to the resist composition in the absence of a resist protective film. In this embodiment, the surfactant has a propensity to segregate on the surface of a resist film for achieving a function of minimizing water penetration or leaching. The surfactant is also effective for preventing water-soluble components from being leached out of the resist film for minimizing any damage to the exposure tool. The surfactant becomes solubilized during alkaline development following exposure and PEB, and thus forms few or no foreign matter which become defects. The preferred surfactant is a polymeric surfactant which is insoluble or substantially insoluble in water, but soluble in alkaline developer, also referred to as “hydrophobic resin” in this sense, and especially which is water repellent and enhances water sliding.

Examples of the polymeric surfactants include those containing repeat units of at least one type selected from formulae (7A) to (7E).

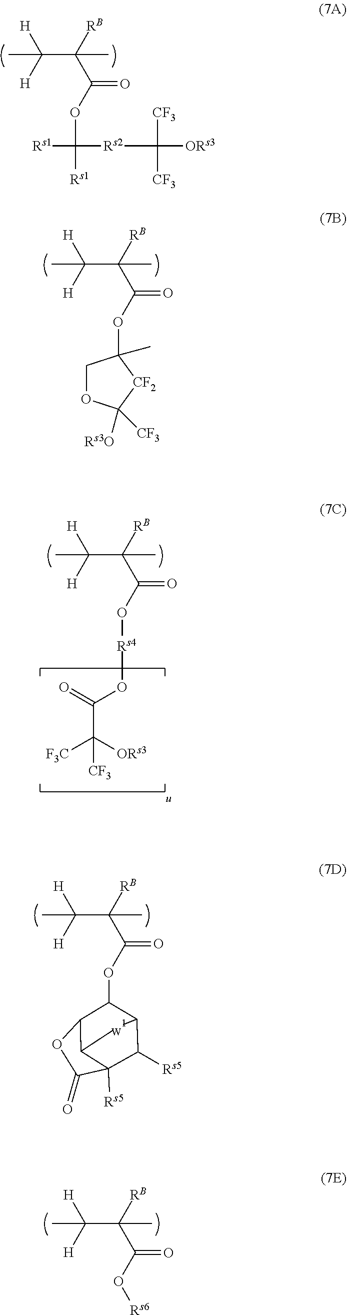

In formulae (7A) to (7E), R^{B} is hydrogen, fluorine, methyl or trifluoromethyl. W is —CH₂—, —CH₂CH₂— or —O—, or two separate —H. R^{s1} is each independently hydrogen or a C₁-C₁₀ hydrocarbyl group. R^{s2} is a single bond or a C₁-C₅ straight or branched hydrocarbylene group. R^{s3} is each independently hydrogen, a C₁-C₁₅ hydrocarbyl or fluorinated hydrocarbyl group, or an acid labile group. When R^{s3} is a hydrocarbyl or fluorinated hydrocarbyl group, an ether bond or carbonyl moiety may intervene in a carbon-carbon bond. R^{s4} is a C₁-C₂₀ (u+1)-valent hydrocarbon or fluorinated hydrocarbon group. u is an integer of 1 to 3. R^{s5} is each independently hydrogen or a group: —C(═O)—O—R^{sa}. R^{sa} is a C₁-C₂₀ fluorinated hydrocarbyl group. R^{s6} is a C₁-C₁₅ hydrocarbyl or fluorinated hydrocarbyl group in which an ether bond or carbonyl moiety may intervene in a carbon-carbon bond.

The hydrocarbyl group represented by R^{s1} is preferably saturated while it may be straight, branched or cyclic. Examples thereof include C₁-C₁₀ alkyl groups such as methyl, ethyl, n-propyl, isopropyl, n-butyl, isobutyl, sec-butyl, tert-butyl, n-pentyl, n-hexyl, n-heptyl, n-octyl, n-nonyl and n-decyl, and C₃-C₁₀ cyclic saturated hydrocarbyl groups such as cyclopropyl, cyclobutyl, cyclopentyl, cyclohexyl, adamantyl and norbornyl. Inter alia, C₁-C₆ groups are preferred.

The hydrocarbylene group represented by R^{s2} is preferably saturated while it may be straight, branched or cyclic. Examples thereof include methylene, ethylene, propylene, butylene, and pentylene.

The hydrocarbyl group represented by R^{s3} or R^{s6} may be saturated or unsaturated and straight, branched or cyclic. Examples thereof include saturated hydrocarbyl groups and aliphatic unsaturated hydrocarbyl groups such as alkenyl and alkynyl groups, with the saturated hydrocarbyl groups being preferred. Suitable saturated hydrocarbyl groups include those exemplified for the hydrocarbyl group represented by R^{s1} as well as n-undecyl, n-dodecyl, tridecyl, tetradecyl, and pentadecyl. Examples of the fluorinated hydrocarbyl group represented by R^{s3} or R^{s6} include the foregoing hydrocarbyl groups in which some or all carbon-bonded hydrogen atoms are substituted by fluorine atoms. In these groups, an ether bond or carbonyl moiety may intervene in a carbon-carbon bond as mentioned above.

Examples of the acid labile group represented by R^{s3} include groups of the above formulae (L1) to (L4), trialkylsilyl groups in which each alkyl moiety has 1 to 6 carbon atoms, and C₄-C₂₀ oxoalkyl groups.

The (u+1)-valent hydrocarbon or fluorinated hydrocarbon group represented by R^{s4} may be straight, branched or cyclic, and examples thereof include the foregoing hydrocarbyl or fluorinated hydrocarbyl groups from which “u” number of hydrogen atoms are eliminated.

The fluorinated hydrocarbyl group represented by R^{s2} is preferably saturated while it may be straight, branched or cyclic. Examples thereof include the foregoing hydrocarbyl groups in which some or all hydrogen atoms are substituted by fluorine atoms. Illustrative examples include trifluoromethyl, 2,2,2-trifluoroethyl, 3,3,3-trifluoro-1-propyl, 3,3,3-trifluoro-2-propyl, 2,2,3,3-tetrafluoropropyl, 1,1,1,3,3,3-hexafluoroisopropyl, 2,2,3,3,4,4,4-heptafluorobutyl, 2,2,3,3,4,4,5,5-octafluoropentyl, 2,2,3,3,4,4,5,5,6,6,7,7-dodecafluoroheptyl, 2-(perfluorobutyl)ethyl, 2-(perfluorohexyl)ethyl, 2-(perfluorooctyl)ethyl, and 2-(perfluorodecyl)ethyl.

Examples of the repeat units having any of formulae (7A) to (7E) are shown below, but not limited thereto. Herein R^{B} is as defined above.

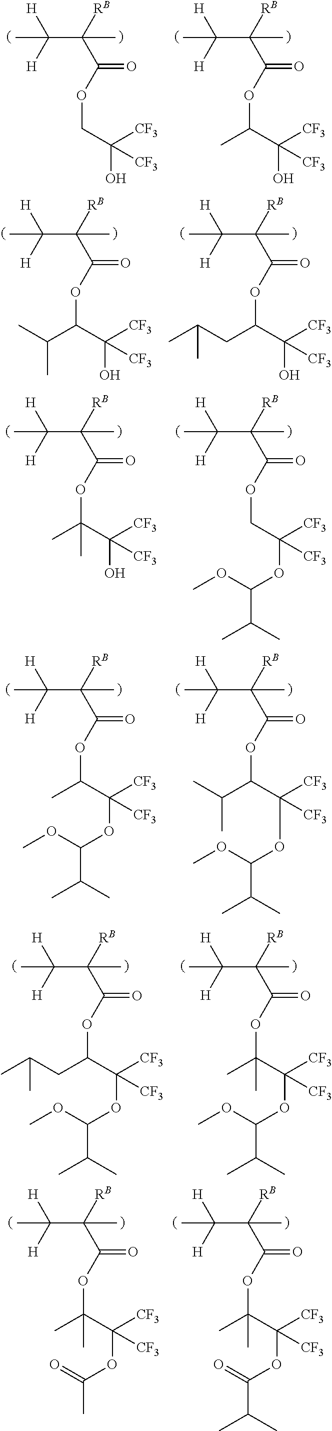

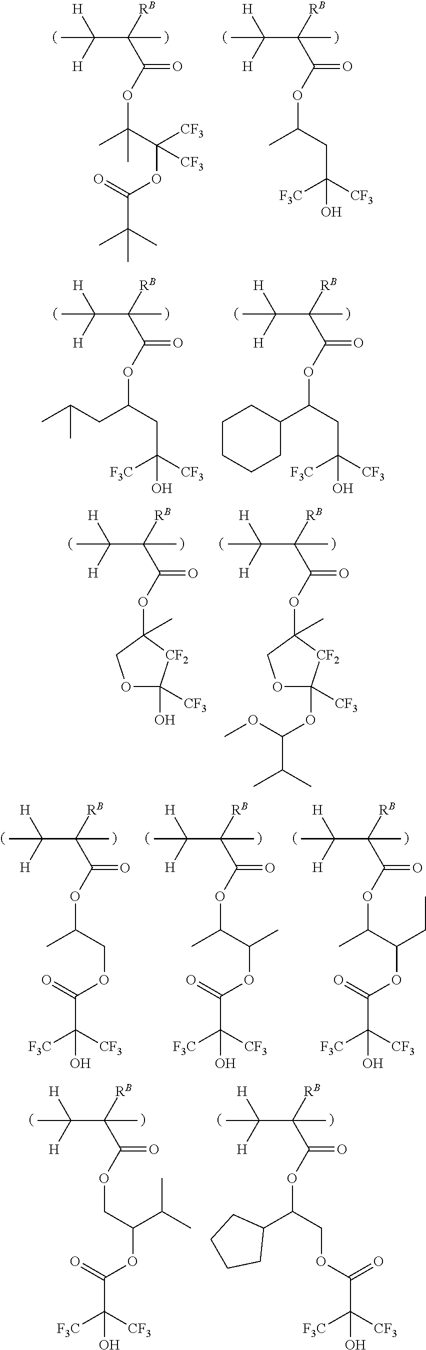

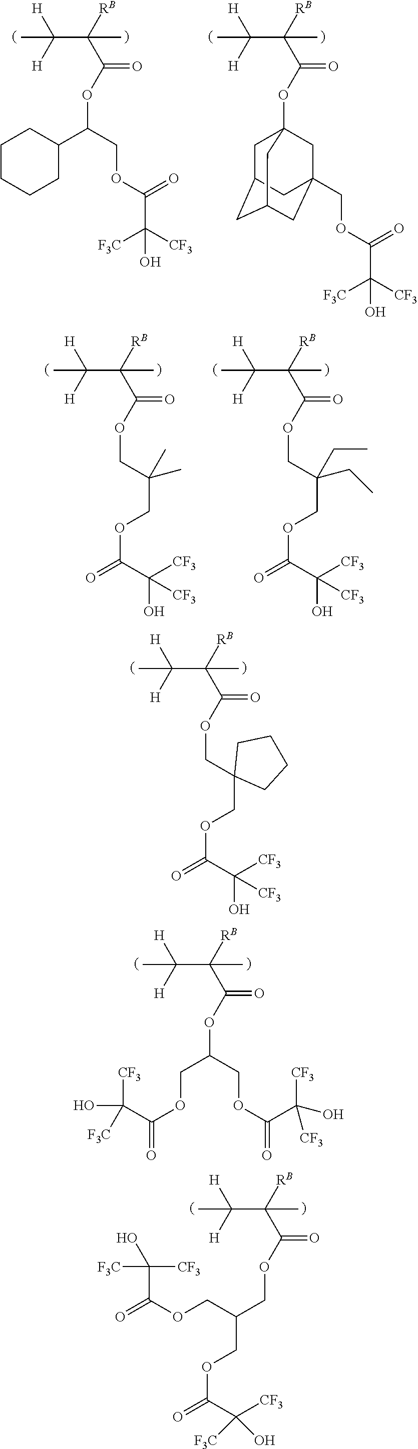

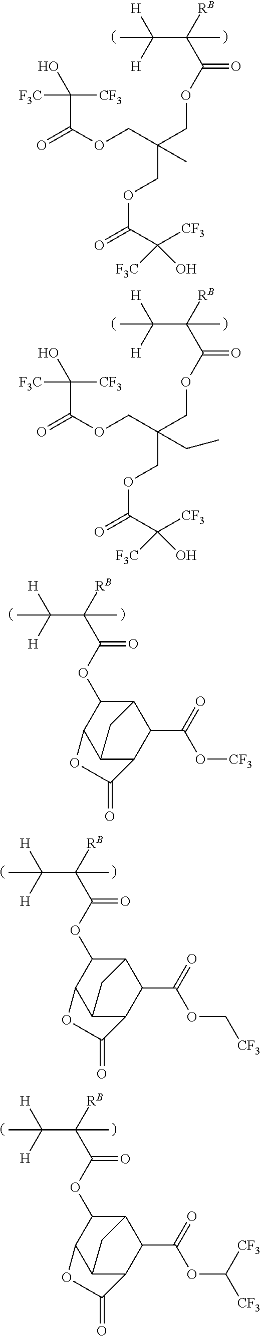

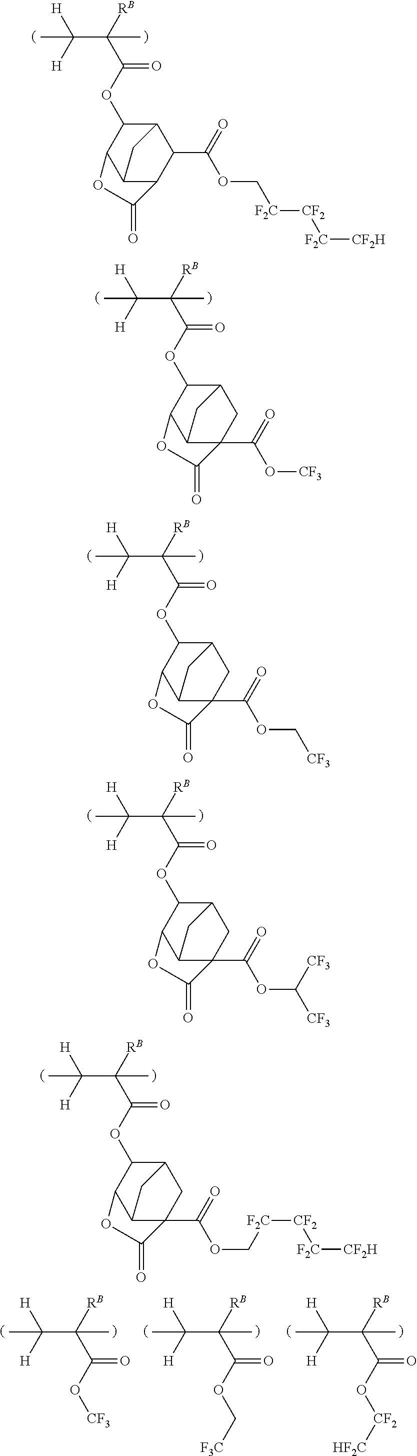

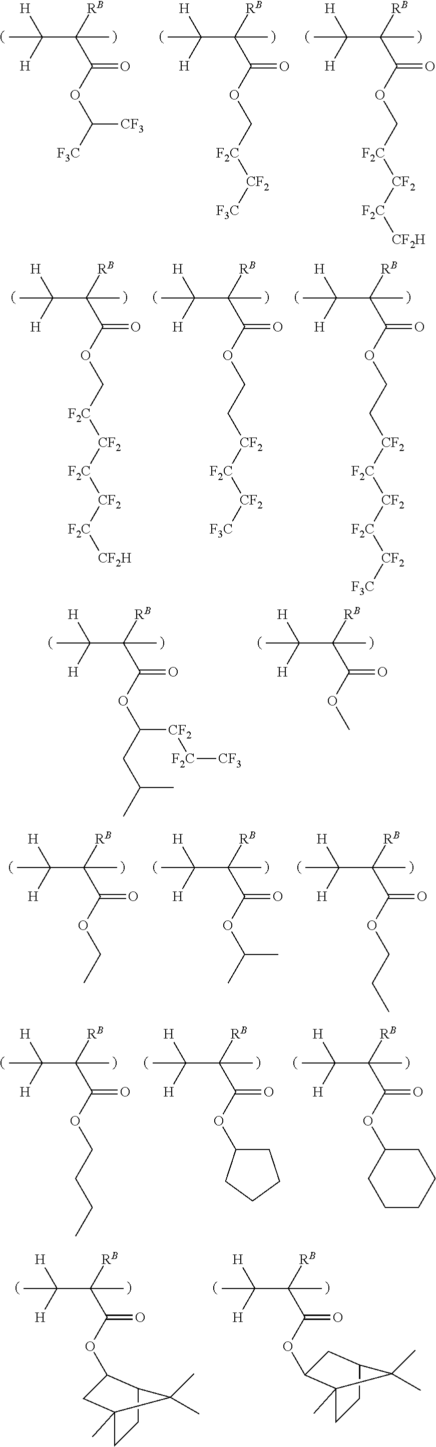

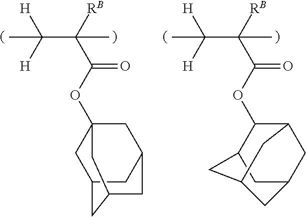

The polymeric surfactant may further contain repeat units other than the repeat units having formulae (7A) to (7E). Typical other repeat units are those derived from methacrylic acid and α-trifluoromethylacrylic acid derivatives. In the polymeric surfactant, the content of the repeat units having formulae (7A) to (7E) is preferably at least 20 mol %, more preferably at least 60 mol %, most preferably 100 mol % of the overall repeat units.

The polymeric surfactant preferably has a Mw of 1,000 to 500,000, more preferably 3,000 to 100,000. A Mw/Mn is preferably 1.0 to 2.0, more preferably 1.0 to 1.6.

The polymeric surfactant may be synthesized, for example, by dissolving an unsaturated bond-containing monomer or monomers, from which the repeat units having formulae (7A) to (7E) and optional other repeat units are derived, in an organic solvent, adding a radical initiator, and heating for polymerization. Suitable organic solvents used herein include toluene, benzene, THF, diethyl ether, and dioxane. Examples of the polymerization initiator used herein include AIBN, 2,2′-azobis(2,4-dimethylvaleronitrile), dimethyl 2,2-azobis(2-methylpropionate), benzoyl peroxide, and lauroyl peroxide. The reaction temperature is preferably 50 to 100° C. The reaction time is preferably 4 to 24 hours. The acid labile group that has been incorporated in the monomer may be kept as such, or the polymerization may be followed by protection or partial protection.

During the synthesis of the polymeric surfactant, any of well-known chain transfer agents such as dodecylmercaptan and 2-mercaptoethanol may be used for the purpose of adjusting molecular weight. An appropriate amount of the chain transfer agent is 0.01 to 10 mol % based on the total moles of monomers to be polymerized.

When the resist composition of the invention contains the surfactant (F), it is preferably used in an amount of 0.1 to 50 parts by weight, and more preferably 0.5 to 10 parts by weight per 80 parts by weight of the base polymer (A). As long as the amount of the surfactant (F) is at least 0.1 part by weight, the receding contact angle of resist film surface with water is fully improved. As long as the amount of the surfactant is up to 50 parts by weight, the dissolution rate of resist film surface in developer is so low that the resulting small-size pattern may maintain a sufficient height. The surfactant (F) may be used alone or in admixture of two or more.

### (G) Other Components

The resist composition of the invention may further comprise other components (G), for example, a compound which is decomposed with an acid to generate another acid (i.e., acid amplifier compound), organic acid derivative, fluorinated alcohol, and a compound with Mw≤3,000 adapted to change its solubility in developer under the action of acid (i.e., dissolution inhibitor). The acid amplifier compound is described in JP-A 2009-269953 and JP-A 2010-215608. The acid amplifier compound is preferably used in an amount of 0 to 5 parts by weight, more preferably 0 to 3 parts by weight per 80 parts by weight of the base polymer (A). An extra amount of the acid amplifier compound can make the acid diffusion control difficult and cause degradations to resolution and pattern profile. With respect to the organic acid derivative, fluorinated alcohol and dissolution inhibitor, reference should be made to JP-A 2009-269953 and JP-A 2010-215608.

### [Pattern Forming Process]

Another embodiment of the invention is a pattern forming process comprising the steps of applying the resist composition defined above onto a substrate to form a resist film thereon, exposing the resist film to high-energy radiation, and developing the exposed resist film in a developer.

The substrate used herein may be selected from, for example, substrates for IC fabrication, e.g., Si, SiO₂, SiN, SiON, TiN, WSi, BPSG, SOG, and organic antireflective coating, and substrates for mask circuit fabrication, e.g., Cr, CrO, CrON, MoSi₂, and SiO₂.

The resist composition is first applied onto a substrate by a suitable coating technique such as spin coating. The coating is prebaked on a hotplate preferably at a temperature of 60 to 150° C. for 1 to 10 minutes, more preferably at 80 to 140° C. for 1 to 5 minutes to form a resist film of 0.05 to 2 μm thick.

Then the resist film is exposed patternwise to high-energy radiation, for example, KrF excimer laser, ArF excimer laser, EB or EUV. On use of KrF or ArF excimer laser or EUV, the resist film is exposed through a mask having the desired pattern, preferably in a dose of 1 to 200 mJ/cm², more preferably 10 to 100 mJ/cm². On use of EB, a pattern may be written directly or through a mask having the desired pattern, preferably in a dose of 1 to 300 μC/cm², more preferably 10 to 200 μC/cm².

The exposure may be performed by conventional lithography whereas the immersion lithography of holding a liquid having a refractive index of at least 1.0, typically water between the resist film and the projection lens may be employed if desired. In the case of immersion lithography, a protective film which is insoluble in water may be formed on the resist film.

While the water-insoluble protective film serves to prevent any components from being leached out of the resist film and to improve water slippage at the film surface, it is generally divided into two types. The first type is an organic solvent-strippable protective film which must be stripped, prior to alkaline development, with an organic solvent in which the resist film is not dissolvable. The second type is an alkali-soluble protective film which is soluble in an alkaline developer so that it can be removed simultaneously with the removal of solubilized regions of the resist film. The protective film of the second type is preferably of a material comprising a polymer having a 1,1,1,3,3,3-hexafluoro-2-propanol residue (which is insoluble in water and soluble in an alkaline developer) as a base in an alcohol solvent of at least 4 carbon atoms, an ether solvent of 8 to 12 carbon atoms or a mixture thereof. Alternatively, the aforementioned surfactant which is insoluble in water and soluble in an alkaline developer may be dissolved in an alcohol solvent of at least 4 carbon atoms, an ether solvent of 8 to 12 carbon atoms or a mixture thereof to form a material from which the protective film of the second type is formed.

After the exposure, the resist film may be baked (PEB). For example, PEB can be performed on a hotplate preferably at 60 to 150° C. for 1 to 5 minutes, and more preferably at 80 to 140° C. for 1 to 3 minutes.

Finally, development is carried out using as the developer an aqueous alkaline solution, such as a 0.1 to 5 wt %, preferably 2 to 3 wt %, aqueous solution of tetramethylammonium hydroxide (TMAH), this being done by a conventional method such as dip, puddle, or spray development for a period of 0.1 to 3 minutes, and preferably 0.5 to 2 minutes. In this way the exposed region of resist film is dissolved away, forming the desired pattern on the substrate.

After formation of the resist film, deionized water rinsing may be carried out for extracting the acid generator and the like from the film surface or washing away particles, or after exposure, rinsing may be carried out for removing water droplets left on the resist film.

A pattern may also be formed by a double patterning process. The double patterning process includes a trench process of processing an underlay to a 1:3 trench pattern by a first step of exposure and etching, shifting the position, and forming a 1:3 trench pattern by a second step of exposure for forming a 1:1 pattern; and a line process of processing a first underlay to a 1:3 isolated left pattern by a first step of exposure and etching, shifting the position, processing a second underlay formed below the first underlay by a second step of exposure through the 1:3 isolated left pattern, for forming a half-pitch 1:1 pattern.

In the pattern forming process of the invention, an alkaline aqueous solution is often used as the developer. Instead, the negative tone development technique wherein the unexposed region of resist film is dissolved in an organic solvent developer is also applicable. In the organic solvent development, the organic solvent used as the developer is preferably selected from 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, ethyl phenylacetate, benzyl formate, phenylethyl formate, methyl 3-phenylpropionate, benzyl propionate, and 2-phenylethyl acetate. These organic solvents may be used alone or in admixture of two or more.

### EXAMPLES

Synthesis Examples, Examples, and Comparative Examples of the invention are given below by way of illustration and not by way of limitation.

### [1] Synthesis of Base Polymer

Monomers of the structure shown below were used in the synthesis of base polymers.

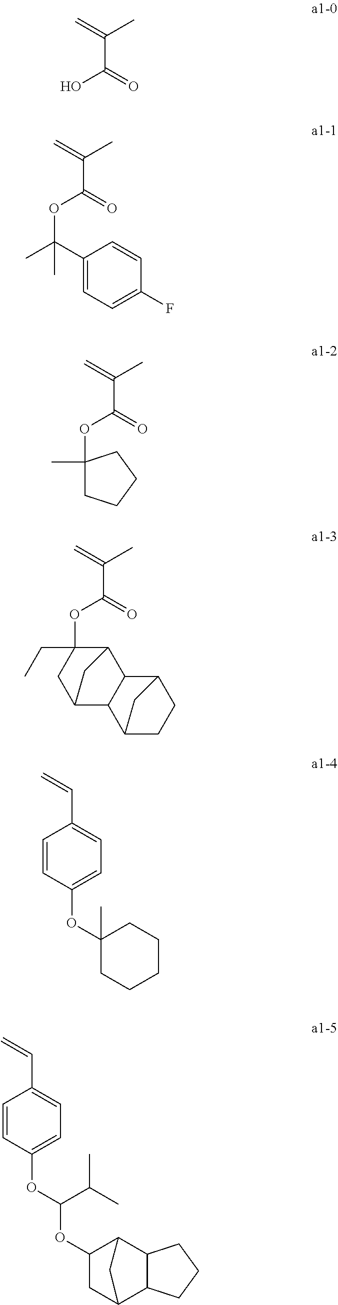

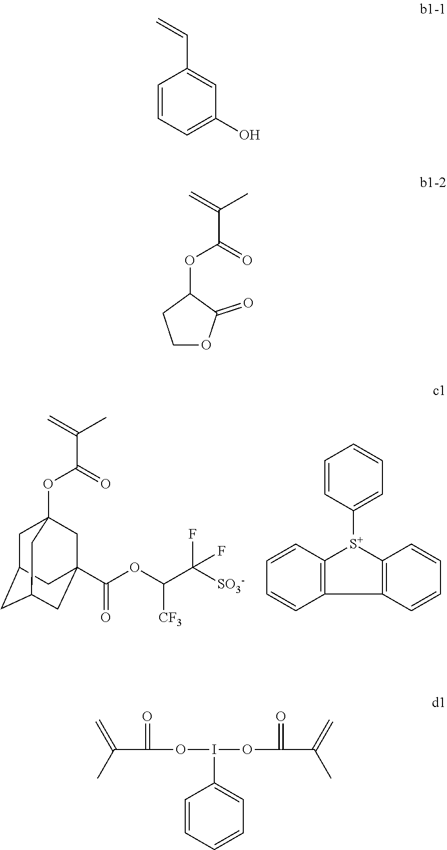

(synthesized with reference to JP-A 2018-095853)

### [Synthesis Example 1] Synthesis of Polymer P-1

A flask under nitrogen atmosphere was charged with 1.9 g of Monomer a1-0, 50.1 g of Monomer a1-1, 16.2 g of Monomer b2-1, 49.7 g of Monomer c1, 3.96 g of V-601 (manufactured by Fujifilm Wako Pure Chemical Corp.), and 127 g of MEK to form a monomer/initiator solution. Another flask under nitrogen atmosphere was charged with 46 g of MEK, which was heated at 80° C. with stirring. The monomer/initiator solution was added dropwise to the MEK over 4 hours. At the end of addition, the polymerization solution was continuously stirred for 2 hours while maintaining the temperature at 80° C. The polymerization solution was cooled to room temperature, after which it was added dropwise to 2,000 g of hexane with vigorous stirring. The precipitate was collected by filtration. The precipitate was washed twice with 600 g of hexane and vacuum dried at 50° C. for 20 hours, obtaining Polymer P-1 as white powder. Amount 119.4 g, yield 98%. Polymer P-1 had a Mw of 10,900 and a Mw/Mn of 1.82. It is noted that Mw is measured by GPC versus polystyrene standards using DMF solvent.

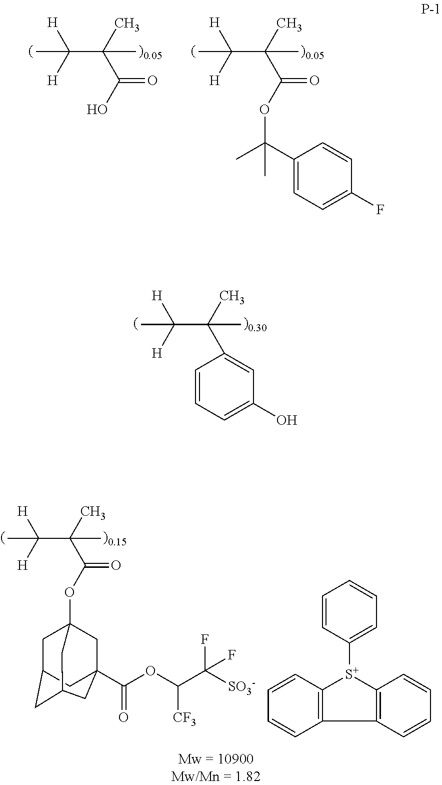

### [Synthesis Examples 2 to 14] Synthesis of Polymers P-2 to P-14

Polymers P-2 to P-14, shown in Table 1, were synthesized by the same procedure as in Synthesis Example 1 except that the type and amount (blending ratio) of monomers were changed.

TABLE 1

| | | | | In- | | In- | | In- | | In- | | In- | | |
|---|---|---|---|---|---|---|---|---|---|---|---|---|---|---|
| | | | | corpo- | | corpo- | | corpo- | | corpo- | | corpo- | | |
| | | | | ration | | ration | | ration | | ration | | ration | | |
| | | | | ratio | | ratio | | ratio | | ratio | | ratio | | |
| | | Polymer | Unit 1 | (mol %) | Unit 2 | (mol %) | Unit 3 | (mol %) | Unit 4 | (mol %) | Unit 5 | (mol %) | Mw | Mw/Mn |
| Synthesis | 1 | P-1 | a1-0 | 5 | a1-1 | 50 | b1-1 | 30 | c1 | 15 | — | — | 10,900 | 1.82 |
| Example | 2 | P-2 | a1-0 | 5 | a1-2 | 50 | b1-1 | 30 | c1 | 15 | — | — | 10,700 | 1.81 |
| | 3 | P-3 | a1-0 | 5 | a1-1 | 25 | a1-2 | 25 | b1-1 | 35 | c1 | 10 | 10,100 | 1.79 |
| | 4 | P-4 | a1-0 | 5 | a1-3 | 50 | b1-1 | 30 | c1 | 15 | — | — | 9,800 | 1.77 |
| | 5 | P-5 | a1-0 | 5 | a1-4 | 50 | b1-1 | 30 | c1 | 15 | — | — | 10,700 | 1.81 |
| | 6 | P-6 | a1-0 | 5 | a1-5 | 55 | b1-1 | 30 | c1 | 10 | — | — | 9,900 | 1.77 |
| | 7 | P-7 | a1-0 | 5 | a1-1 | 40 | b1-1 | 30 | b1-2 | 15 | c1 | 10 | 9,700 | 1.84 |
| | 8 | P-8 | a1-0 | 5 | a1-1 | 55 | b1-1 | 40 | — | — | — | — | 7,300 | 1.76 |
| | 9 | P-9 | a1-0 | 5 | a1-2 | 55 | b1-1 | 40 | — | — | — | — | 7,400 | 1.74 |
| | 10 | P-10 | a1-0 | 5 | a1-3 | 55 | b1-2 | 40 | — | — | — | — | 7,700 | 1.78 |
| | 11 | P-11 | a1-1 | 55 | b1-1 | 30 | c1 | 15 | — | — | — | — | 10,200 | 1.81 |
| | 12 | P-12 | a1-1 | 60 | b1-1 | 40 | — | — | — | — | — | — | 15,200 | 1.97 |
| | 13 | P-13 | a1-1 | 50 | b1-1 | 30 | c1 | 15 | d1 | 5 | — | — | 18,700 | 1.99 |
| | 14 | P-14 | a1-1 | 55 | b1-1 | 40 | d1 | 5 | — | — | — | — | 17,800 | 1.98 |

### [2] Preparation of Resist Composition

### Examples 1-1 to 1-14 and Comparative Examples 1-1 to 1-6

A resist composition (R-01 to R-14) was prepared by dissolving a polymer (P-1 to P-14), hypervalent iodine compound (I-1 to I-3), photoacid generator (PAG-1), and quencher (Q-1, Q-2) in a solvent containing 0.01 wt % of surfactant (PF-636, manufactured by OMNOVA Solutions Inc.) in accordance with the formulation shown in Table 2, and filtering the solution through a Teflon® filter with a pore size of 0.2 μm. Separately, a comparative resist composition (CR-01 to CR-06) was prepared by mixing a polymer, photoacid generator, sensitivity modifier, hypervalent iodine compound, solvent, and 0.01 wt % of surfactant (PF-636, manufactured by OMNOVA Solutions Inc.) in accordance with the formulation shown in Table 3, and filtering the solution through a Teflon® filter with a pore size of 0.2 μm.

TABLE 2

| | | | | Photoacid | | Hypervalent | | |
|---|---|---|---|---|---|---|---|---|
| | | Resist | Polymer | generator | Quencher | iodine compound | Solvent 1 | Solvent 2 |
| | | composition | (pbw) | (pbw) | (pbw) | (pbw) | (pbw) | (pbw) |
| Example | 1-1 | R-01 | P-1 | — | Q-1 | I-1 | PGMEA | DAA |
| | | | (80) | | (6.2) | (2.4) | (2200) | (900) |
| | 1-2 | R-02 | P-2 | — | Q-1 | I-1 | PGMEA | DAA |
| | | | (80) | | (6.2) | (2.4) | (2200) | (900) |
| | 1-3 | R-03 | P-3 | — | Q-1 | I-1 | PGMEA | DAA |
| | | | (80) | | (6.2) | (2.4) | (2200) | (900) |
| | 1-4 | R-04 | P-4 | — | Q-1 | I-1 | PGMEA | DAA |
| | | | (80) | | (6.2) | (2.4) | (2200) | (900) |
| | 1-5 | R-05 | P-5 | — | Q-1 | I-1 | PGMEA | DAA |
| | | | (80) | | (6.2) | (2.4) | (2200) | (900) |
| | 1-6 | R-06 | P-6 | — | Q-1 | I-1 | PGMEA | DAA |
| | | | (80) | | (6.2) | (2.4) | (2200) | (900) |
| | 1-7 | R-07 | P-7 | — | Q-1 | I-1 | PGMEA | DAA |
| | | | (80) | | (6.2) | (2.4) | (2200) | (900) |
| | 1-8 | R-08 | P-8 | PAG-1 | Q-1 | I-1 | PGMEA | DAA |
| | | | (80) | (19.0) | (6.2) | (2.4) | (2200) | (900) |
| | 1-9 | R-09 | P-9 | PAG-1 | Q-1 | I-1 | PGMEA | DAA |
| | | | (80) | (19.0) | (6.2) | (2.4) | (2200) | (900) |
| | 1-10 | R-10 | P-10 | PAG-1 | Q-1 | I-1 | PGMEA | DAA |
| | | | (80) | (19.0) | (6.2) | (2.4) | (2200) | (900) |
| | 1-11 | R-11 | P-1 | — | Q-2 | I-1 | PGMEA | DAA |
| | | | (80) | | (2.6) | (2.4) | (2200) | (900) |
| | 1-12 | R-12 | P-1 | — | Q-1 | I-2 | PGMEA | DAA |
| | | | (80) | | (6.2) | (2.7) | (2200) | (900) |
| | 1-13 | R-13 | P-1 | — | Q-1 | I-3 | PGMEA | DAA |
| | | | (80) | | (6.2) | (3.1) | (2200) | (900) |
| | 1-14 | R-14 | P-1 | — | Q-1 | I-1 | HBM | — |
| | | | (80) | | (6.2) | (2.4) | (3100) | |

TABLE 3

| | | | | Photoacid | | Hypervalent | | |
|---|---|---|---|---|---|---|---|---|
| | | Resist | Polymer | generator | Quencher | iodine compound | Solvent 1 | Solvent 2 |
| | | composition | (pbw) | (pbw) | (pbw) | (pbw) | (pbw) | (pbw) |
| Comparative | 1-1 | CR-01 | P-1 | — | Q-1 | — | PGMEA | DAA |
| Example | | | (80) | | (6.2) | | (2200) | (900) |
| | 1-2 | CR-02 | P-8 | PAG-1 | Q-1 | — | PGMEA | DAA |
| | | | (80) | (19.0) | (6.2) | | (2200) | (900) |
| | 1-3 | CR-03 | P-11 | PAG-1 | Q-1 | I-1 | PGMEA | DAA |
| | | | (80) | (19.0) | (6.2) | (6.2) | (2200) | (900) |
| | 1-4 | CR-04 | P-12 | PAG-1 | Q-1 | I-1 | PGMEA | DAA |
| | | | (80) | (19.0) | (6.2) | (6.2) | (2200) | (900) |
| | 1-5 | CR-05 | P-13 | — | Q-1 | — | PGMEA | DAA |
| | | | (80) | | (6.2) | | (2200) | (900) |
| | 1-6 | CR-06 | P-14 | PAG-1 | Q-1 | — | PGMEA | DAA |
| | | | (80) | (19.0) | (6.2) | | (2200) | (900) |

The components in Tables 2 and 3 are identified below.

Organic solvent:
PGMEA (propylene glycol monomethyl ether acetate)
DAA (diacetone alcohol)
HBM (methyl 2-hydroxyisobutyrate)

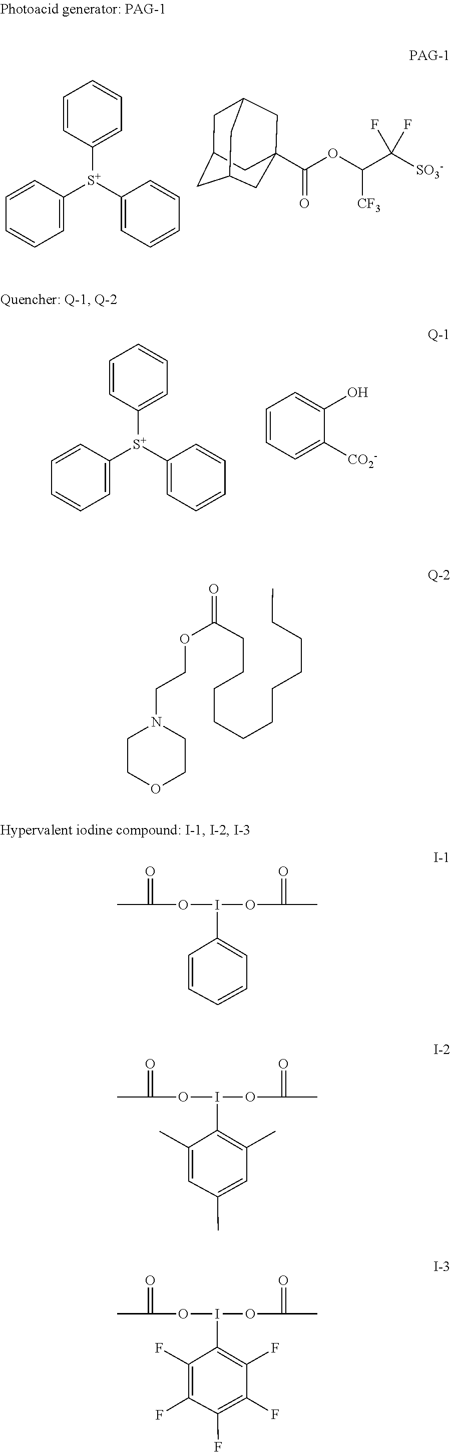

### [3] EUV Lithography Test

### Examples 2-1 to 2-14 and Comparative Examples 2-1 to 2-6

Each of the resist compositions (R-01 to R-14, CR-01 to CR-06) was spin coated on a silicon substrate having a 20-nm coating of silicon-containing spin-on hard mask SHB-A940 (Shin-Etsu Chemical Co., Ltd., silicon content 43 wt %) and prebaked on a hotplate at 100° C. for 60 seconds to form a resist film of 50 nm thick. Using an EUV scanner NXE3300 (ASML, NA 0.33, a 0.9/0.6, dipole illumination), the resist film was exposed to EUV through a mask bearing a line-and-space (LS) pattern having a width of 18 nm and a pitch of 36 nm (on-wafer size) while changing the dose at a pitch of 1 mJ/cm² and the focus at a pitch of 0.020 μm. The resist film was baked (PEB) at the temperature shown in Tables 4 and 5 for 60 seconds. This was followed by puddle development in a 2.38 wt % TMAH aqueous solution for 30 seconds, rinsing with a surfactant-containing rinse fluid, and spin drying. A positive LS pattern was obtained. The LS pattern was observed under CD-SEM (CG6300, Hitachi High-Technologies Corp.) and evaluated for sensitivity, LWR, depth of focus (DOF), and collapse limit by the following methods. The results are shown in Tables 4 and 5.

### [Evaluation of Sensitivity]

The optimum dose Eop (mJ/cm²) which provided an LS pattern with a line width of 18 nm and a pitch of 36 nm was determined and reported as sensitivity.

### [Evaluation of LWR]

For the LS pattern formed by exposure at the optimum dose Eop, the line width was measured at 10 longitudinally spaced apart points, from which a 3-fold value (3a) of the standard deviation (a) was determined and reported as LWR. A smaller value of 3a indicates a pattern having small roughness and uniform line width.

### [Evaluation of DOF]

As an index of DOF, a range of focus which provided an LS pattern with a size of 18 nm±10% (i.e., 16.2 to 19.8 nm) was determined. A greater value indicates a wider DOF.

### [Evaluation of Collapse Limit of Line Pattern]

For the LS pattern formed by exposure at the dose corresponding to the optimum focus, the line width was measured at 10 longitudinally spaced apart points. The minimum line size above which lines could be resolved without collapse was determined and reported as collapse limit. A smaller value indicates better collapse limit.

TABLE 4

| | | Resist | PEB temp. | Eop | LWR | DOF | Collapse limit |
|---|---|---|---|---|---|---|---|
| | | composition | (° C.) | (mJ/cm²) | (nm) | (nm) | (nm) |
| Example | 2-1 | R-01 | 105 | 38 | 2.6 | 120 | 12.6 |
| | 2-2 | R-02 | 110 | 39 | 2.7 | 110 | 12.4 |
| | 2-3 | R-03 | 110 | 41 | 2.8 | 120 | 13.2 |
| | 2-4 | R-04 | 105 | 38 | 2.9 | 120 | 13 |
| | 2-5 | R-05 | 110 | 40 | 2.9 | 110 | 12.8 |
| | 2-6 | R-06 | 100 | 36 | 3.1 | 130 | 12.6 |
| | 2-7 | R-07 | 105 | 39 | 2.9 | 110 | 12.2 |
| | 2-8 | R-08 | 105 | 38 | 2.9 | 120 | 12.7 |
| | 2-9 | R-09 | 110 | 40 | 3.0 | 120 | 13.1 |
| | 2-10 | R-10 | 105 | 39 | 2.9 | 130 | 13 |
| | 2-11 | R-11 | 105 | 40 | 2.7 | 120 | 12.6 |
| | 2-12 | R-12 | 105 | 39 | 2.7 | 110 | 12.7 |
| | 2-13 | R-13 | 105 | 38 | 2.8 | 120 | 13.1 |
| | 2-14 | R-14 | 105 | 38 | 2.6 | 130 | 12.2 |

TABLE 5

| | | Resist | PEB temp. | Eop | LWR | DOF | Collapse limit |
|---|---|---|---|---|---|---|---|
| | | composition | (° C.) | (mJ/cm²) | (nm) | (nm) | (nm) |
| Comparative | 2-1 | CR-01 | 105 | 37 | 3.8 | 90 | 15.0 |
| Example | 2-2 | CR-02 | 105 | 36 | 3.6 | 100 | 15.5 |
| | 2-3 | CR-03 | 105 | 40 | 3.9 | 100 | 14.9 |
| | 2-4 | CR-04 | 105 | 39 | 3.7 | 90 | 14.8 |
| | 2-5 | CR-05 | 105 | 50 | 4.3 | 80 | 15.5 |
| | 2-6 | CR-06 | 105 | 48 | 4.2 | 90 | 15.8 |

It is demonstrated in Tables 4 and 5 that resist compositions within the scope of the invention exhibit a high resolution and improved lithography properties.

Japanese Patent Application No. 2024-039577 is incorporated herein by reference. Although some preferred embodiments have been described, many modifications and variations may be made thereto in light of the above teachings. It is therefore to be understood that the invention may be practiced otherwise than as specifically described without departing from the scope of the appended claims.

1. A resist composition comprising (A) a base polymer containing a polymer comprising repeat units having an acid labile group and repeat units having a carboxy group, (B) an organic solvent, and (C) a hypervalent iodine compound having formula (1): 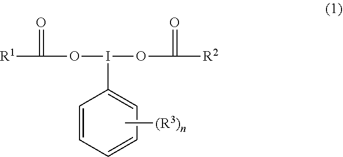
wherein n is an integer of 0 to 5,
R¹ and R² are each independently halogen or a C₁-C₁₀ hydrocarbyl group which may contain a heteroatom, R¹ and R² may bond together to form a ring with the carbon atoms to which they are attached and the intervenient atoms, and
R³ is halogen or a C₁-C₄₀ hydrocarbyl group which may contain a heteroatom.

2. The resist composition of claim 1 wherein the repeat unit having an acid labile group is represented by formula (a1) or (a2): 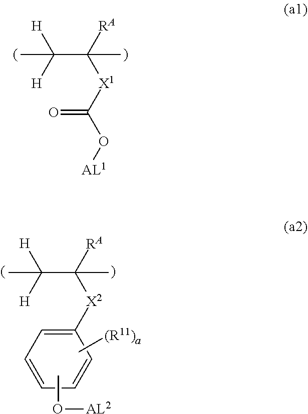
wherein a is an integer of 0 to 4,
R^{A} is each independently hydrogen, fluorine, methyl or trifluoromethyl,
X¹ is a single bond, phenylene group, naphthylene group or *—C(═O)—O—X¹¹—, X¹¹ is a C₁-C₁₀ saturated hydrocarbylene group which may contain at least one selected from a hydroxy moiety, ether bond, ester bond and lactone ring, a phenylene group or naphthylene group,
X² is a single bond or *—C(═O)—O—,
* designates a point of attachment to the carbon atom in the backbone,
R¹¹ is halogen or a C₁-C₂₀ hydrocarbyl group which may contain a heteroatom, and
AL¹ and AL² are each independently an acid labile group.

3. The resist composition of claim 1 wherein the repeat unit having a carboxy group is represented by formula (b): 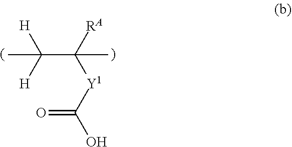
wherein R^{A} is hydrogen, fluorine, methyl or trifluoromethyl,
Y¹ is a single bond, phenylene group, naphthylene group or *—C(═O)—O—Y¹¹—, Y¹¹ is a C₁-C₁₀ saturated hydrocarbylene group which may contain at least one selected from a hydroxy moiety, ether bond, ester bond and lactone ring, a phenylene group or naphthylene group, and * designates a point of attachment to the carbon atom in the backbone.

4. The resist composition of claim 1 wherein the polymer further comprises at least one selected from repeat units having formulae (c1) and (c2): 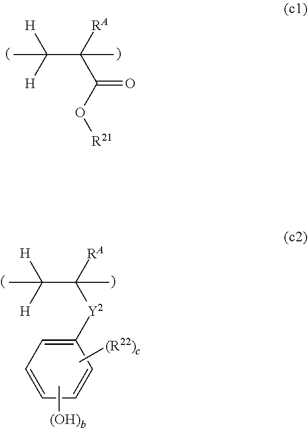
wherein b is 1 or 2, c is an integer of 0 to 4,
R^{A} is each independently hydrogen, fluorine, methyl or trifluoromethyl,
Y² is a single bond or *—C(═O)—O—, * designates a point of attachment to the carbon atom in the backbone,
R²¹ is a C₁-C₂₀ group containing at least one selected from hydroxy, cyano, carbonyl, ether bond, ester bond, sulfonic ester bond, carbonate bond, lactone ring, sultone ring, and carboxylic anhydride (—C(═O)—O—C(═O)—), and
R²² is halogen or a C₁-C₂₀ hydrocarbyl group which may contain a heteroatom.

5. The resist composition of claim 1 wherein the polymer further comprises repeat units having a photoacid generating group.

6. The resist composition of claim 1, further comprising (D) a photoacid generator.

7. The resist composition of claim 1, further comprising (E) a quencher.

8. The resist composition of claim 1, further comprising (F) a surfactant.

9. A pattern forming process comprising the steps of applying the resist composition of claim 1 to a substrate to form a resist film thereon, exposing the resist film to KrF excimer laser, ArF excimer laser, electron beam or extreme ultraviolet and developing the exposed resist film in a developer.
